SEQUENCE LISTING (15 sequences)

SEQ ID NO: 1 - protein, 31 aa, Homo sapiens
HAEGTFTSDVSSYLEGQAAKEFIAWLVKGRG

SEQ ID NO: 2 - protein, 29 aa, Homo sapiens
HAEGTFTSDVSSYLEGQAAKEFIAWLVKG

SEQ ID NO: 3 - protein, 39 aa, Heloderma suspectum
HGEGTFTSDLSKQMEEEAVRLFIEWLKNGGPSSGAPPPS

SEQ ID NO: 4 - protein, 29 aa, Homo sapiens
HSQGTFTSDYSKYLDSRRAQDFVQWLMNT

SEQ ID NO: 5 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 6 - protein, 18 aa, Artificial Sequence
  mimetic peptide
DWFKAFYDKVAEKFKEAF

SEQ ID NO: 7 - protein, 37 aa, Artificial Sequence
  mimetic peptide
DWLKAFYDKVAEKLKEAFPDWAKAAYDKAAEKAKEAA

SEQ ID NO: 8 - protein, 22 aa, Artificial Sequence
  mimetic peptide
PVLDLFRELLNELLEALKQKLK

SEQ ID NO: 9 - protein, 34 aa, Artificial Sequence
  synthetic peptide
HAEGTFTSDVSSYLEGEAVKEFIAWLVEKVKEFL

SEQ ID NO: 10 - protein, 34 aa, Artificial Sequence
  synthetic peptide
HAQGTFTSDYSKYLDSKAAREFVQWLLEKVKEFL

SEQ ID NO: 11 - protein, 34 aa, Artificial Sequence
  synthetic peptide
HAEGTFTSDLSKQMEEEAVREFIEWLKNKVLEFL

SEQ ID NO: 12 - protein, 44 aa, Artificial Sequence
  Formula I
XXXGTXTXDXXXXXXXXXXXXFXXXLXXXXXXXXXXXXXXXXXX

SEQ ID NO: 13 - protein, 29 aa, Heloderma suspectum
HGEGTFTSDLSKQMEEEAVRLFIEWLKNG

SEQ ID NO: 14 - protein, 20 aa, Artificial Sequence
  mimetic peptide
ELREKLEAWFELFREFLERF

SEQ ID NO: 15 - protein, 26 aa, Artificial Sequence
  mimetic peptide
EVRSKLEEWFAAFREFAEEFLARLKS

(19) United States
(12) Patent Application Publication
(10) Pub. No.: US 2015/0344540 A1
(43) Pub. Date: Dec. 3, 2015

(54) NOVEL GLP-1 RECEPTOR AGONISTS WITH CHOLESTEROL EFFLUX ACTIVITY

(71) Applicant: NOVO NORDISK A/S, Bagsvaerd (DK)

(72) Inventors: Christian Wenzel Tornoee, Lyngby (DK); Henning Thoegersen, Farum (DK); Bidda Charlotte Rolin, Naerum (DK); Janos Tibor Kodra, Koebenhavn OE (DK); Salka Elboel Rasmussen, Vaerloese (DK); Jesper Lau, Farum (DK)

(21) Appl. No.: 14/653,976

(22) PCT Filed: Dec. 19, 2013
(86) PCT No.: PCT/EP2013/077357

§ 371 Date: Jun. 19, 2015

Related U.S. Application Data
(60) Provisional application No. 61/740,469, filed on Dec. 21, 2012.

(30) Foreign Application Priority Data
Dec. 19, 2012 (EP) 12198295.3

(51) Int. Cl. C07K 14/605

(52) U.S. Cl. CPC C07K 14/605

(57) ABSTRACT

The present invention provides novel glucagon-like protein-1 (GLP-1) receptor agonist compounds that promote cholesterol efflux. The ABCA1-mediated cholesterol efflux present invention also provides compositions comprising the novel glucagon-like protein-1(GLP-1) receptor agonist compounds, and relates to the use of said compounds in therapy, to methods of treatment comprising administration of said compounds to patients, and to the use of said compounds in the manufacture of medicaments.

### SEQUENCE LISTING

A Sequence Listing of 10.3 kilobytes was created on 19 Dec. 2012, and is incorporated herein by reference.

### TECHNICAL FIELD

The present invention provides novel glucagon-like protein-1(GLP-1) receptor agonist compounds that promote cholesterol efflux. The present invention also provides compositions comprising the novel glucagon-like protein-1(GLP-1) receptor agonist compounds, and relates to the use of said compounds in therapy, to methods of treatment comprising administration of said compounds to patients, and to the use of said compounds in the manufacture of medicaments.

### BACKGROUND

Diabetes is a group of chronic diseases characterized by hyperglycemia. Modern medical care uses a vast array of lifestyle and pharmaceutical interventions aimed at preventing and controlling hyperglycemia. Despite control of hyperglycemia, the primary cause of morbidity and mortality in diabetic patients throughout the world remains to be cardiovascular disease (CVD) (Valensi P, Picard S: Lipids, lipid-lowering therapy and diabetes complications; Diabetes Metab. 2011 37 (1) 15-24).

The major cause of CVD is accelerated atherosclerosis; a chronic inflammatory disease in the arterial wall (Farmer J A, Liao J: Evolving concepts of the role of high-density lipoprotein in protection from atherosclerosis; Curr. Atheroscier. Rep. 2011 13 (2) 107-14). Atherosclerotic plaque formation is initiated by the deposition of excess cholesterol, primarily derived from plasma low density lipoprotein (LDL), in the inner layer of the vascular wall (i.e. tunica intima). The cholesterol-containing LDL is oxidized or otherwise modified and taken up by resident macrophages, thus turning these into lipid-filled foam cells. Cholesterol can be effluxed from the vascular wall, from macrophages and foam cells via specific transporters (ABCA-1 and ABCG-1) to high density lipoprotein (HDL) particles and transported to the liver for excretion via the bile. This process is known as reverse cholesterol transport (RCT).

Research suggests that low levels of HDL or dysfunctional HDL are correlated to increased risk of CVD. Type 2 diabetes patients have often reduced HDL levels and dysfunctional HDL and are thus at elevated risk for CVD (Farbstein D, Levy A P: HDL dysfunction in diabetes: causes and possible treatments; Expert. Rev. Cardiovasc. Ther. 2012 10 (3) 353-61; Barter P HDL-C: Role as a risk modifier; Atheroscler. Suppl. 2011 12 (3) 267-70). Furthermore, recent studies also suggest that the ability of plasma to exert reverse cholesterol transport (measured as the cholesterol efflux capacity) determines the risk for cardiovascular disease (Khera A V, Cuchel M, de la Llera-Moya M, Rodrigues A, Burke M F, Jafri K, French B C, Phillips J A, Mucksavage M L, Wilensky R L, Mohler E R, Rothblat G H, Rader D J: Cholesterol efflux capacity, high-density lipoprotein function, and atherosclerosis; N. Engl. J. Med. 2011 13 364 (2) 127-35).

Studies have suggested that intravenous injection of ApoA-I or its variant ApoA-I Milano in human subjects was able to significantly regress atherosclerosis (Nissen S E, Tsunoda T, Tuzcu E M, Schoenhagen P, Cooper C J, Yasin M, Eaton G M, Lauer M A, Sheldon W S, Grines C L, Halpern S, Crowe T, Blankenship J C, Kerensky R; JAMA 2003 5 290 (17) 2292-2300). Treatment with ApoA-I has however considerable limitations due to high cost and requirement for intravenous injection or infusion making it suitable mainly only for acute treatment.

Glucagon-like protein-1 (GLP-1) receptor agonist peptides have been shown to have several beneficial effects in diabetes patients such as improved blood glucose control, lowering of glycated hemoglobin A1c (HbA1c) and lowering of body weight with an overall improved lipid profile in type 2 diabetic patients. There are currently three approved GLP-1 receptor agonists on the market Victoza®, Byetta® and Bydureon®. These compounds are most often taken in combination with one or several other blood glucose lowering agents. Despite the current treatment available many diabetes patients still suffer both from poor blood glucose control and elevated HbA1c, and also have an increased risk of cardiovascular disease.

GLP-1 and Exendin-4 do not possess cholesterol efflux activity despite when bound to the N-terminal of the GLP-1 receptor (Underwood et al, J. Biol. Chem. 2010 285 723; and Runge et al, J. Biol. Chem. 2008 283 11340), they do adopt an alpha helical conformation which in part is amphipathic. One possible explanation why this effect is not present is that the amphipathic part of the helix is approximately 13 residues long, and this is too short to promote cholesterol efflux activity.

Novel GLP-1 receptor agonists providing both good blood HbA1c control and increased cholesterol efflux activity would be of great benefit for diabetes patients since this would address the unmet need for a treatment reducing the risk of cardiovascular disease in patients with diabetes. Thus, the development of new peptides that are both GLP-1 receptor agonists and have the capability of promoting cholesterol efflux constitute a very promising therapeutically approach.

### SUMMARY OF THE INVENTION

The present invention relates to novel GLP-1 receptor agonist compounds that promote cholesterol efflux, to compositions thereof, to the use of said compounds in therapy, to methods of treatment comprising administration of said compounds to patients, and to the use of said compounds in the manufacture of medicaments.

In one embodiment, the present invention provides novel GLP-1 receptor agonists which in an alpha helical conformation comprise an amphipathic helix.

In another embodiment, the present invention provides novel GLP-1 receptor agonist peptide which in an alpha helical conformation comprise an amphipathic helix, wherein said peptide has cholesterol efflux activity with an Eₘₐₓ of at least 65% of that of L-4F, and a potency measured as EC₅₀, that is better than the potency of L-4F, when measured according to the methods described in Example 6.

In a third embodiment, the present invention provides novel GLP-1 receptor agonists which in an alpha helical conformation comprise an amphipathic helix holding 15 or more amino acid residues.

In a fourth embodiment, the GLP-1 receptor agonists of the invention promote cholesterol efflux, and also bind to and activate the GLP-1 receptor.

In a fifth embodiment, the present invention provides novel GLP-1 receptor agonists which have cholesterol efflux activity.

In a further embodiment, the present invention provides novel GLP-1 receptor agonists comprising an amino acid sequence of Formula I:

X₇-X₈-X₉-Gly-Thr-X₁₂-Thr-X₁₄-Asp-X₁₆-X₁₇-X₁₈-X₁₉-X₂₀-X₂₁-X₂₂-X₂₃-X₂₄-X₂₅-X₂₆-X₂₇-Phe-X₂₉-X₃₀-X₃₁-Leu-X₃₃-X₃₄-X₃₅-X₃₆-X₃₇-X₃₈-X₃₉-X₄₀-X₄₁-X₄₂-X₄₃-X₄₄-X₄₅-X₄₆-X₄₇-X₄₈-X₄₉-X₅₀

wherein,

X₇ represents His, or desamino-His;

X₈ represents Ala, Gly, Ser, or Aib;

X₉ represents Glu, Asp, Gln, or His;

X₁₂ represents Phe, Tyr, or Leu;

X₁₄ represents Ser, Asn, or His;

X₁₆ represents Val, Tyr, Leu, Ile, or Met;

X₁₇ represents Ser, or Thr;

X₁₈ represents Ser, Lys, Arg, Glu, Asn, or Gln;

X₁₉ represents Tyr, or Gln;

X₂₀ represents Leu, Met, or Tyr;

X₂₁ represents Glu, Asp, or Gln;

X₂₂ represents Gly, Ser, Glu, Pro, Lys, or Aib;

X₂₃ represents Gln, Glu, Lys, Trp, or Asp;

X₂₄ represents Ala, Aib, Lys, or Arg;

X₂₅ represents Ala, Val, Leu, Ile, or Aib;

X₂₆ represents Lys, Asn, Glu, Arg, His, Gly, Val, or Gln;

X₂₇ represents Glu, Asp, Gln, Ala, His, Gly, Arg, Lys, Aib, or Leu;

X₂₉ represents Ile, or Val;

X₃₀ represents Ala, Val, Gln, Ile, Trp, Aib, Glu, Arg, or Lys;

X₃₁ represents Trp, Gln, Lys, or His;

X₃₃ represents Val, Ile, Leu, Thr, Arg, or Lys;

X₃₄-X₃₅-X₃₆-X₃₇-X₃₈-X₃₉ represents Subsequence 1, composed by the following amino acid residues “Glu-Lys-Aib-Lys-Glu-Phe”; or in which Subsequence 1, one, two or three amino acid residues have been substituted for

[Asn, Gln, Lys, His, Gly, Arg, or Asp] in position X₃₄;

[Arg, Ala, His, Gln, Asn, or Aib] in position X₃₅;

[Gly, Val, Leu, Phe, Ile, Trp, Tyr, Ala, Met, or His] in position X₃₆;

[Arg, Ala, Leu, Gly, His, Gln, Asn, Aib, Ile, Val, or Phe] in position X₃₇;

[Asp, His, Gln, Ser, Gly, Asn, or Thr] in position X₃₈; and/or

[Trp, Ala, Glu, Leu, Val, Gly, His, Lys, Ser, Thr, Tyr, Aib, Ile, or Met] in position X₃₉; and

X₄₀ represents Gly, Leu, Phe, Val, His, Tyr, or amide, or X₄₀ is absent;

X₄₁ represents Glu, Asp, Ala, Gly, Lys, or amide, or X₄₁ is absent;

X₄₂ represents Leu, Pro, Lys, Arg, or amide, or X₄₂ is absent;

X₄₃ represents Leu, Pro, Val, or amide, or X₄₃ is absent;

X₄₄ represents Lys, or amide, or X₄₄ is absent;

X₄₅ represents Glu, or amide, or X₄₅ is absent;

X₄₆ represents Phe, Ile, or amide, or X₄₆ is absent;

X₄₇ represents Ile, or amide, or X₄₇ is absent;

X₄₈ represents Ala, or amide, or X₄₈ is absent;

X₄₉ represents Trp, or amide, or X₄₉ is absent;

X₅₀ represents amide, or X₅₀ is absent;

provided, however:

if X₄₁, X₄₂, X₄₃, X₄₄, X₄₅, X₄₆, X₄₇, X₄₈, X₄₉ or X₅₀ is absent, then each amino acid residue downstream is also absent;

and pharmaceutically acceptable salts, amides, esters, acids or prodrugs thereof.

In a yet further embodiment, the GLP-1 receptor agonist peptides represent peptides of Formula I, as described above, wherein X₇-X₃₅ represents Exendin-4(1-29) with up to 10 amino acid substitutions.

In a still further embodiment, the GLP-1 receptor agonist peptides represent peptides of Formula I, as described above, wherein X₇-X₃₅ represents GLP-1(7-35) with up to 10 amino acid substitutions.

In a still further embodiment, the GLP-1 receptor agonist peptides represent peptides of Formula I, as described above, wherein X₇-X₃₅ represents glucagon peptide (1-29) with up to 10 amino acid substitutions.

The present invention further relates to GLP-1 receptor agonist peptides of the present invention, wherein said GLP-1 receptor agonist peptide has been C-terminally fused to an ApoA-I mimetic peptide.

The present invention also provides a pharmaceutical composition comprising the GLP-1 receptor agonist peptide of the invention.

The present invention also provides GLP-1 receptor agonist peptides, for use as a medicament.

The present invention also provides peptides, compositions thereof, uses and methods for treating or preventing diseases including, but not limited to, diabetes, obesity, dyslipidemia, inflammatory diseases, hypercholesterolemia, cardiovascular disease, atherosclerosis, endothelial dysfunction, macrovascular disorders or microvascular disorders.

In one embodiment, the peptides of the present invention reduce HbA1C, while also promoting cholesterol efflux.

The present invention provides novel GLP-1 receptor agonist compounds, which surprisingly combine the effects of GLP-1 receptor binding and activation, with promotion of cholesterol efflux, and therefore provides a novel therapeutic concept that addresses both reduction of blood glucose, and prevention or treatment of cardiovascular complications, found in patients with diabetes.

The present invention may also solve further problems that will be apparent from the disclosure of the exemplary embodiments.

### DETAILED DISCLOSURE OF THE INVENTION

The present invention relates to novel, dual acting peptides which have the advantage of targeting both diabetes and cardiovascular disease, i.e., they both reduce blood glucose and reduce risk of cardiovascular disease. This is an unmet need in diabetes care, as many diabetes patients have high risk of cardiovascular disease.

The present invention provides novel GLP-1 receptor agonist peptides, which surprisingly combine the effects of GLP-1 receptor binding and activation, with promotion of cholesterol efflux, and therefore provide a novel therapeutic concept that addresses both reduction of blood glucose, and prevention or treatment of cardiovascular complications, found in patients with diabetes.

The present invention provides peptides which surprisingly exert cholesterol efflux activity at physiological relevant concentrations. This is surprising, since the native GLP-1 and Exendin-4 peptides do not exert cholesterol efflux activity at physiological relevant concentrations (i.e. EC₅₀ is above 10 μM, see FIG. 2).

More specifically, the present invention relates to novel GLP-1 receptor agonist peptides which are dual-acting, i.e. they both bind to and activate the GLP-1 receptor, and also exert cholesterol efflux. These dual acting peptides exert their effect directly, or by binding to lipids or through mediators. The mediators include, but are not limited to HDL, ABC transporters, and mediators for oxidation and inflammation.

The GLP-1 receptor agonist peptides of the present invention may be used for treatment or prevention in diabetic or obese patients with additional complications such as hyperlipidimia and hypercholesterolemia, cardiovascular disease, endothelial dysfunction, a macrovascular disorder, microvascular disorder, or atherosclerosis.

### GLP-1 Receptor Agonist

A receptor agonist may be defined as a peptide that binds to a receptor and elicits a response typical of the natural ligand. Thus, for example, a “GLP-1 receptor agonist” or “GLP-1 receptor agonist peptide” is defined as a compound which is capable of binding to the GLP-1 receptor and capable of activating it.

### GLP-1 Peptides and Analogues

The term “GLP-1”, “GLP-1 peptide” or “hGLP-1” as used herein refers to the human Glucagon-Like Peptide-1 (GLP-1(7-37)), the sequence of which is included in the sequence listing as SEQ ID 1, or an analogue thereof. The peptide having the sequence of SEQ ID 1 may also be designated “native” GLP-1.

The Homo sapiens GLP-1(7-37) sequence is:

HAEGTFTSDV SSYLEGQAAK EFIAWLVKGR G (SEQ ID 1); and

the Homo sapiens GLP-1(7-35) sequence is:

HAEGTFTSDV SSYLEGQAAK EFIAWLVKG (SEQ ID 2).

The term “GLP-1 analogue” or “analogue of GLP-1” as used herein refers to a peptide, or a compound, which is a variant of GLP-1(7-37) (SEQ ID 1) or of GLP-1(7-35) (SEQ ID 2).

In the sequence listing, the first amino acid residue (i.e. histidine) of SEQ ID 1 is assigned No. 1. However, in what follows—according to established practice in the art—this histidine residue is referred to as No. 7, and subsequent amino acid residues are numbered accordingly, ending with glycine No. 37. Therefore, generally, any reference herein to an amino acid residue number or a position number of the GLP-1(7-37) sequence is to the sequence starting with His at position 7 and ending with Gly at position 37.

For the purposes of numbering in Formula I (SEQ ID 12), the same principle is used, i.e. start position X₇ corresponds to histidine in position 7 of native GLP-1 and ends in position X₃₇, corresponding to position 37 in native GLP-1(7-37) sequence. However, as for the sequence listing, the first amino acid residue of SEQ ID 12 (histidine or X₇) is assigned No. 1.

The same principle applies for numbering of GLP-1(7-35): histidine residue is referred to as No. 7, and subsequent amino acid residues are numbered accordingly, ending with glycine No. 35.

GLP-1 analogues of the invention may be described by reference to i) the number of the amino acid residue in native GLP-1(7-37) or GLP-1(7-35), which corresponds to the amino acid residue which is changed (i.e., the corresponding position in native GLP-1), and to ii) the actual change.

In other words, a GLP-1 analogue is a GLP-1(7-37) or GLP-1(7-35) peptide in which a number of amino acid residues have been changed when compared to native GLP-1(7-37) (SEQ ID 1) or GLP-1(7-35) (SEQ ID 2). These changes may represent, independently, one or more amino acid substitutions, additions, and/or deletions.

The following are non-limiting examples of suitable analogue nomenclature.

Analogues “comprising” certain specified changes may comprise further changes, when compared to SEQ ID 1 or SEQ ID 2. In a particular embodiment, the analogue “has” the specified changes.

As is apparent from the above examples, amino acid residues may be identified by their full name, their one-letter code, and/or their three-letter code. These three ways are fully equivalent.

The expressions “a position equivalent to” or “corresponding position” may be used to characterise the site of change in a variant GLP-1(7-37) sequence by reference to native GLP-1(7-37) (SEQ ID 1) or GLP-1(7-35) (SEQ ID 2). Equivalent or corresponding positions, as well as the number of changes, are easily deduced, e.g. by simple handwriting and eyeballing; and/or a standard protein or peptide alignment program may be used, such as “align” which is based on a Needleman-Wunsch algorithm. This algorithm is described by Needleman, S. B. and Wunsch, C. D.; Journal of Molecular Biology 1970 48: 443-453; and the align program by Myers and W. Miller in “Optimal Alignments in Linear Space” CABIOS (computer applications in the biosciences) 1988 4 11-17. For the alignment, the default scoring matrix BLOSUM62 and the default identity matrix may be used, and the penalty for the first residue in a gap may be set at −12, or preferably at −10, and the penalties for additional residues in a gap at −2, or preferably at −0.5.

For an overview, GLP-1 receptor agonist peptides may be aligned as illustrated in Table 1 below:

TABLE 1

| | |
|---|---|
| | 7 10 20 30 |
| GLP-1(7-35) | HAE GTFTSDVSSY LEGQAAKEFI AWLVKG |
| Exendin-4(1-29) | HGE GTFTSDLSKQ MEEEAVRLFI EWLKNG |
| Glucagon (1-29) | HSQ GTFTSDYSKY LDSRRAQDFV QWLMNT |
| | 1 4 14 24 |

The term “GLP-1 peptide”, as e.g. used in the context of this invention, refers to a compound which comprises a series of amino acids interconnected by amide (or peptide) bonds.

A GLP-1 receptor agonist peptide of the invention may be any polypeptide comprising (i.e. including, but not limited to) an amino acid sequence as described herein, and thus may comprise additional amino acid residues.

In one embodiment the GLP-1 receptor agonist peptide of the invention comprise at least 31 amino acids.

In another embodiment, the GLP-1 receptor agonist peptide of the invention is composed of at least 32, or at least 33, or at least 34 amino acids.

In a third embodiment, the GLP-1 receptor agonist peptide of the invention holds of from 30 to 46 amino acid residues.

In a fourth embodiment, the GLP-1 receptor agonist peptide of the invention holds of from 32 to 42 amino acid residues.

In a fifth embodiment, the GLP-1 receptor agonist peptide of the invention holds of from 33 to 40 amino acid residues.

In a still further particular embodiment the GLP-1 receptor agonist peptide consists of amino acids interconnected by peptide bonds.

Amino acids are molecules containing an amine group and a carboxylic acid group, and, optionally, one or more additional groups, often referred to as a side chain.

The term “amino acid” includes proteinogenic amino acids (encoded by the genetic code, including natural amino acids, and standard amino acids), as well as non-proteinogenic (not found in proteins, and/or not coded for in the standard genetic code), and synthetic amino acids. Thus, the amino acids may be selected from the group of proteinogenic amino acids, non-proteinogenic amino acids, and/or synthetic amino acids.

Non-limiting examples of amino acids which are not encoded by the genetic code are gamma-carboxyglutamate, ornithine (Orn), norleucine (Nle) and phosphoserine. Non-limiting examples of synthetic amino acids are Aib (α-aminoisobutyric acid), β-alanine, and des-amino-histidine (alternative name imidazopropionic acid, abbreviated Imp).

In what follows, all amino acids of the GLP-1 peptide, for which the optical isomer is not stated, are to be understood to mean the L-isomer (unless otherwise specified).

The GLP-1 receptor agonist peptides of the invention have GLP-1 activity. This term refers to the ability to bind to the GLP-1 receptor and initiate a signal transduction pathway resulting in an insulinotropic action or other physiological effects as is known in the art. For example, the analogues of the invention can be tested for GLP-1 activity using the assay described in Example 2 (in vitro), or in Example 7 (in vivo) herein.

### Exenatide

Exenatide is a commercial incretin mimetic for the treatment of diabetes mellitus type 2, which is manufactured and marketed by Amylin Pharmaceuticals and Eli Lilly & Co. Exenatide is based on Exendin-4, a hormone found in the saliva of the Gila monster (Heloderma suspectum), that displays biological properties similar to human GLP-1. U.S. Pat. No. 5,424,286 relates i.e. to a method of stimulating insulin release in a mammal by administration of Exendin-4(1-39) (SEQ ID 3).

The Gila monster Exendin-4(1-39) sequence is:

HGEGTFTSDL SKQMEEEAVR LFIEWLKNGG PSSGAPPPS (SEQ ID 3),

while the sequence of Exendin-4(1-29) is:

HGEGTFTSDL SKQMEEEAVR LFIEWLKNG (SEQ ID 13)

For the purposes of numbering in Formula I (SEQ ID 12), the start position X₇ of Formula I corresponds to histidine in position 1 of Exendin-4 (SEQ ID 3 and 13), and ends in position X₃₇, corresponding to position 31 in Exendin-4 sequence (SEQ ID 3 and 13), or position X₄₅, corresponding to position 39 in Exendin-4 (SEQ ID 3).

However, as for the sequence listing, the first amino acid residue of SEQ ID 3 and SEQ ID 13 (histidine or X₇ of Formula I) is assigned No. 1. Exendin-4 amino acids positions 1 to 39 in SEQ ID 3 are to be the same as amino acid positions X₇ to X₄₅ of Formula I. Likewise, amino acid positions 1 to 29 of Exendin-4 (1-29) (SEQ ID 13) are to be the same as amino acid positions X₇ to X₃₅. For the purposes of numbering in Formula I, the first amino acid residue (histidine) of SEQ ID Nos. 3 and 13 is assigned X₇.

### Glucagon Peptide

Concerning position numbering in glucagon compounds, and as defined herein, any amino acid substitution, deletion, and/or addition is indicated relative to the sequences of native human glucagon (1-29) (SEQ ID 4). For the purposes of numbering in Formula I (SEQ ID 12), the start position X₇ of Formula I corresponds to histidine in position 1 of native glucagon (SEQ ID 4) and ends in position X₃₅, corresponding to position 29 in native glucagon. However, as for the sequence listing, the first amino acid residue of native glucagon (histidine or X₇ of Formula I is assigned No. 1. Human glucagon amino acids positions 1 to 29 are herein to be the same as amino acid positions X₇ to X₃₅ of Formula I.

The human (Homo sapiens) glucagon (1-29) sequence is

HSQGTFTSDY SKYLDSRRAQ DFVQWLMNT (SEQ ID 4).

### Peptide

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

In the context of this invention, common rules for peptide nomenclature based on the three or one letter amino acid code apply. Briefly, the central portion of the amino acid structure is represented by the three letter code (e.g. Ala, Lys) or one letter code (e.g. A, K) and L-configuration is assumed, unless D-configuration is specifically indicated by “D-” followed by the three letter code (e.g. D-Ala, D-Lys). A substituent at the amino group replaces one hydrogen atom and its name is placed before the three letter code, whereas a C-terminal substituent replaces the carboxylic hydroxy group and its name appears after the three letter code. For example, “acetyl-Gly-Gly-NH₂” represents CH₃—C(═O)—NH—CH₂—C(═O)—NH—CH₂—C(═O)—NH₂. Unless indicated otherwise, amino acids are connected to their neighboring groups by amide bonds formed at the N-2 (α-nitrogen) atom and the C-1 (C═0) carbon atom.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A simple system is used to describe analogues. Formulae of peptide analogues and derivatives thereof are drawn using standard single letter or three letter abbreviations for amino acids used according to IUPAC-IUB nomenclature.

A sequence alignment is a way of arranging the sequences of DNA, RNA, or protein to identify regions of similarity that may be a consequence of functional, structural, or evolutionary relationships between the sequences. Aligned sequences of nucleotide or amino acid residues are typically represented as rows within a matrix. Gaps are inserted between the residues so that identical or similar characters are aligned in successive columns.

In the context of chemical compounds of the present invention, similarity and/or identity may be determined using any suitable computer program and/or algorithm known in the art. A more complete list of available software categorized by algorithm and alignment type is available at sequence alignment software, but common software tools used for general sequence alignment tasks include ClustalW and T-coffee for alignment, and BLAST and FASTA3x for database searching.

The term “Sequence identity (% SI)” as used herein can be calculated using the following formula:

% SI=100%*(Nr of identical residues in pairwise alignment)/(Length of the shortest sequence)

In one embodiment, non-limiting examples of further peptides of the present invention comprise the following sequences:

(SEQ ID 9)

HAEGTFTSDV SSYLEGEAVK EFIAWLVEKV KEFL;

(SEQ ID 10)

HAQGTFTSDY SKYLDSKAAR EFVQWLLEKV KEFL;
and

(SEQ ID 11)

HAEGTFTSDL SKQMEEEAVR EFIEWLKNKV LEFL.

The term “downstream” of an amino acid position means an amino acid or amino acid sequence located to the right of that position when writing the peptide primary structure with the N-terminus to the left and the C-terminus to the right, i.e. amino acid positions of increasing position numbers.

### Apolipoproteins

The term “apolipoproteins” or “apo” or “Apo” refers to any of the several water soluble proteins that combine with lipid to form lipoproteins. These lipoproteins can be separated by size or by flotation densities and generally classified as chylomicrons, VLDL, LDL and HDL. Apolipoproteins include non-exchangeable protein Apo B and exchangeable proteins e.g. Apo A-I, Apo A-II, Apo A-IV, Apo C-I, Apo C-II, Apo C-III, Apo E, and serum amyloid proteins such as serum amyloid A.

The term “apolipoprotein A-I” or “ApoA-I”, refers to a polypeptide comprising 243 amino acids forming N and C-terminal domains. Residues 44-243 of ApoA-I contain the necessary structural determinants for mediating cholesterol efflux via ABCA1 or other ABC transporters. This region of ApoA-I (aa44-243) is comprised of a series of ten amphipathic alpha-helices of 11- or 22-amino acids separated by proline residues. The individual alpha-helical segments of ApoA-I are defined, in part, by the relative distribution of positively charged residues and are designated as Class A or Y. Class A helices possess positively charged amino acid toward the middle of the polar surface in addition to interfacial cationic residues.

The human ApoA-I sequence is as follows:

(SEQ ID 5)

DEPPQSPWDR VKDLATVYVD VLKDSGRDYV SQFEGSALGK
QLNLKLLDNW DSVTSTFSKL REQLGPVTQE FWDNLEKETE
GLRQEMSKDL EEVKAKVQPY LDDFQKKWQE EMELYRQKVE
PLRAELQEGA RQKLHELQEK LSPLGEEMRD RARAHVDALR
THLAPYSDEL RQRLAARLEA LKENGGARLA EYHAKATEHL
STLSEKAKPA LEDLRQGLLP VLESFKVSFL SALEEYTKKL
NTQ.

### ApoA-I Mimetics

The term “mimetic” as used herein is meant to be a molecule that mimics the activity of another molecule, such as the biological activity of the molecule, ApoA-I mimetics thus mimic the effect of full-length ApoA-I.

The term “ApoA-I mimetic”, “ApoA-I mimetic peptide”, “ApoA-I compound”, as used herein refers to an ApoA-I mimetic peptide, an analogue or a derivative of the human ApoA-I consensus peptide, as well as analogues, fusion peptides and derivatives thereof, which maintain ApoA-I activity, i.e. promote cholesterol efflux. The term “ApoA-I mimetics” refers to amphiphatic peptides that can mimic the action of ApoA-I like those known in the art e.g. but not limited to, the peptides, described by Navab et al; Apolipoprotein A-1 Mimetic Peptides, Arterioscler Thromb Vasc Biol. 2005; 25:1325-1331, including, but not limited to those allegedly disclosed by Segrest et al; PROTEINS Structure, Function, and Genetics 15349-359 (1993). Non-limiting examples of ApoA-I mimetics are L-4F, D-4F, SEQ ID 7, SEQ ID 8, SEQ ID 14 and SEQ ID 15.

The term “L-4F” as used herein means an ApoA-I mimetic, i.e. L-4F, also referred to as 4F or L4F, is a synthetic mimetic containing four phenylalanine amino acids. The L-4F (1-18) sequence is DWFKAFYDKV AEKFKEAF (SEQ ID 6).

All amino acids for which the optical isomer is not stated is to be understood to mean the L-isomer. D-4F shall be the same sequence as L-4F, where all amino acids are D amino acids.

Sequences of other non-limiting examples of ApoA-I mimetics are the bihelical 5A mimetic with the following sequences:

DWLKAFYDKV AEKLKEAFPD WAKAAYDKAA EKAKEAA (SEQ ID 7) (known from i.a. WO 2006/044596 and WO 2009/032749);

PVLDLFRELL NELLEALKQK LK (SEQ ID 8) (known from i.a. WO 99/16459);

ELREKLEAWFELFREFLERF (SEQ ID 14); and

EVRSKLEEWFAAFREFAEEFLARLKS (SEQ ID 15) (known from i.a. WO 2009/155366 and WO 2008/115303).

In embodiments of the invention, a maximum of 18 amino acids in the ApoA-I mimetic peptide have been modified. In other embodiments of the invention, a maximum of 15 amino acids in the ApoA-I mimetic peptide have been modified. In yet other embodiments of the invention, a maximum of 12 amino acids in the ApoA-I mimetic peptide have been modified. In yet other embodiments of the invention a maximum of 8 amino acids in the ApoA-I mimetic peptide have been modified. In yet other embodiments of the invention a maximum of 6 amino acids in the ApoA-I mimetic peptide have been modified. In yet other embodiments of the invention a maximum of 5 amino acids in the ApoA-I mimetic peptide have been modified. In yet other embodiments of the invention a maximum of 4 amino acids in the ApoA-I mimetic peptide have been modified. In yet other embodiments of the invention a maximum of 3 amino acids in the ApoA-I mimetic peptide have been modified. In yet other embodiments of the invention a maximum of 2 amino acids in the ApoA-I mimetic peptide have been modified. In yet other embodiments of the invention 1 amino acid in the ApoA-I mimetic peptide has been modified.

### Additional Definitions

The term “alpha helical conformation” as used herein, refers to a specific secondary structure common in many proteins. The alpha helical conformation is a specific conformation where the peptide folds in a repeating pattern in which the backbone carbonyl oxygen of one residue forms a hydrogen bond to a backbone NH four residues later in the peptide sequence, exposing all amino acid side chain atoms to the outside of the helix. A peptide in an alpha helical conformation makes a complete turn every 3.6 amino acid residue.

The term “amphipathic alpha helix” or “amphipathic peptide” or “amphipathic helix” as used herein, refers to a polypeptide sequence that, when adopting a secondary structure that is helical, will have one surface, i.e. one face along the helix axis being polar and comprised primarily of hydrophilic or polar amino acid residues (non-limiting examples are Asp, Glu, Lys, Arg, His, Gly, Ser, Thr, Cys, Asn and Gln) and the other surface along the helix axis being a lipophilic or nonpolar face that comprises primarily hydrophobic amino acid residues (non-limiting examples are Leu, Ala, Ile, Pro, Phe, Trp, Aib, Tyr and Met).

The term “hydrophilic face” or “polar face” as used herein refers to a exposed continuous surface along the helix axis that is comprised primarily of hydrophilic or polar amino acid residues (non-limiting examples are Asp, Glu, Lys, Arg, His, Gly, Ser, Thr, Cys, Asn and Gln)

The term “lipophilic face” or “hydrophobic face” as used herein refers to an exposed continuous surface along the helix axis that is comprised primarily of hydrophobic acid residues (non-limiting examples are Leu, Ala, Ile, Val, Pro, Phe, Trp, Aib and Met).

The term “conservative substitution” as used herein refers to substitution of one peptide amino acid residue with another amino acid residue with similar characteristics such as charge, size, hydrophobicity, hydrophilicity, presence of identical functional group (eg. hydroxyl group) and/or aromaticity, or when both residues are classified as lipophilic amino acid residues (non-limiting examples are Ser with Thr, Lys with Arg, Phe with Trp and Asp with Glu), and includes exchanges within the following four groups:

I. Ala, Ser, Thr, Gly, Cys

II. Asp, Asn, Glu, Gln

III. His, Arg, Lys, Orn

IV. Met, Leu, Ile, Val, Cys, Phe, Tyr, Trp, Pro, Nle

The term “lipophilic amino acid residue” or “hydrophobic amino acid residue” as used herein, refers to an amino acid residue, where the side chain either does not contain any nitrogen or oxygen atoms, or if so, the carbon atom to oxygen- or nitrogen atom ratio is greater than or equal to 7. Non-limiting examples include amino acid residues Ala, Cys, Phe, Ile, Leu, Met, Pro, Val, Trp, Tyr and Aib.

The term “hydrophilic amino acid residue” or “polar amino acid residue” as used herein refers to Gly or Cys or an amino acid residue that does comprise at least one oxygen or nitrogen in the sidechain in a carbon to nitrogen or oxygen ratio of less than or equal to 7. Non-limiting examples include the amino acid residues Cys, Asp, Glu, His, Lys, Asn, Gln, Arg, Ser, Gly, Thr and Tyr.

The term “charged amino acid residue” as used herein refers to an amino acid residue with a side chain which at neutral pH may be charged (non-limiting examples are Asp, Glu, Arg, Lys and His).

The term “negatively charged amino acid residue” or “acidic amino acid residue” as used herein refers to an amino acid residue with a side chain which at neutral pH can have a charge of −1 or less (non-limiting examples are Asp and Glu). The term “positively charged amino acid residue” or “basic amino acid residue” as used herein refers to an amino acid residue with a side chain which at neutral pH can have a charge of +1 or more (non-limiting examples are Arg, Lys and His).

The term “fused C-terminally” in the relation that one peptide has been fused C-terminally to another peptide means that a peptide bond is formed between the backbone C-terminal carboxylic acid of one peptide and the backbone N-terminal amino group of the other peptide.

The term “ABC” or “ATP Binding Casette” refers to multi-domain membrane proteins responsible for the controlled efflux and influx of lipids (e.g. cholesterol and phospholipids) across cellular membranes. ABC transporters are trans-membrane proteins that utilize the energy of adenosine triphosphate (ATP) hydrolysis to carry out certain biological processes including translocation of various substrates across membranes. They transport a wide variety of substrates across extra- and intracellular membranes, including metabolic products, lipids and sterols, and drugs. Proteins are classified as ABC transporters based on the sequence and organization of their ATP-binding cassette (ABC) domain(s).

There are 48 known ABC transporters present in humans, which are classified into seven families by the Human Genome Organization. The ABCA family contains some of the largest transporters (over 2,100 amino acids long). Five of them are located in a cluster in the 17q24 chromosome. These transporters are responsible for the transportation of cholesterol and lipids, among other things. Examples are ABCA1 and ABCA12. The ABCG family also transports lipids, diverse drug substrates, bile, cholesterol, and other steroids. Examples are ABCG1 and ABCG2.

The term “ABCA1” refers to the ATP-binding cassette transporter ABCA1 (member 1 of human transporter sub-family ABCA), also known as the cholesterol efflux regulatory protein (CERP) is a protein which in humans is encoded by the ABCA1 gene. This transporter is a major regulator of cellular cholesterol and phospholipid homeostasis.

### Cholesterol Efflux

Macrophage or foam cells in the artery wall release or export cholesterol to acceptors, such as apolipoproteins and/or HDL or the peptides of the current invention. A compound that mediates cholesterol efflux enhances the release of cholesterol out of the cell and into the extracellular compartment. Cholesterol efflux is often accompanied by the efflux of phospholipids from the cell. The coordinated release of both cholesterol and phospholipids produces HDL in the presence of a suitable lipid acceptor, eg. apolipoprotein or peptide. Therefore, the processes of cholesterol- and phospholipid efflux are linked and synonymous with one another. ABCA1-dependent lipid efflux (or lipid efflux by an ABCA1-dependent pathway) refers to a process whereby apolipoproteins or peptides that facilitate cholesterol efflux, interact with a cell or vesicle and efflux lipid from the cell by a process that is facilitated by the ABCA1 transporter.

The current invention relates to GLP-1 receptor agonist compounds that promote cholesterol efflux. Here we specifically define the term “cholesterol efflux” or “cholesterol efflux activity” as the efflux of cholesterol from a macrophage cell line as described in Example 6. Compounds of the invention show an efficacy measured as Eₘₐₓ of at least 65%, or at least 70%, or at least 75%, or at least 80%, of that of L-4F, and a potency measured as EC₅₀ better than the potency of L-4F measured as described in Example 6. The cholesterol efflux potency can be expressed as the EC₅₀ value.

The EC₅₀ value, defined as the half maximal effective concentration, refers to the concentration of a drug, antibody or toxicant which induces a response halfway between the baseline and maximum after a specified exposure time. It is commonly used as a measure of drug's potency.

Many different equations can be used to derive an EC₅₀. One possible function is:

$$Y = Bottom + \frac{Top - Bottom}{1 + {( \frac{X}{{EC}_{50}} )}^{- Hillcoefficient}}$$

where Y is the observed value, Bottom is the lowest observed value, Top is the highest observed value (which equals Eₘₐₓ), and the Hill coefficient gives the largest absolute value of the slope of the curve.

The term “reverse cholesterol transport” or “reverse cholesterol transport activity” (abbreviated “RCT”) refer to the mediation of cholesterol efflux from cells of the arterial wall to the liver or other steroidogenic organs. The reverse cholesterol transport pathway has three main steps, i) cholesterol efflux, i.e. the initial removal of cholesterol from various pools of peripheral cells; ii) cholesterol esterification by the action of lechitin cholesterol acyltransferase (LCAT), thereby preventing re-entry of effluxed cholesterol into cells; iii) uptake of the cholesteryl ester by HDL and deloivery of the cholesteryl ester complex to liver cells. Enhancement of cholesterol efflux and of reverse cholesterol transport (RCT) is considered an important target for anti-atherosclerotic drug therapy.

The term “physical stability” of the GLP-1 receptor agonist peptide preparation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein preparations is evaluated by means of visual inspection and/or turbidity measurements after exposing the preparation filled in suitable containers (e.g. cartridges or vials) to mechanical/physical stress (e.g. agitation) at different temperatures for various time periods. Visual inspection of the preparations is performed in a sharp focused light with a dark background. The turbidity of the preparation is characterized by a visual score ranking the degree of turbidity for instance on a scale from 0 to 3 (a preparation showing no turbidity corresponds to a visual score 0, and a preparation showing visual turbidity in daylight corresponds to visual score 3). A preparation is classified physically unstable with respect to protein aggregation, when it shows visual turbidity in daylight. Alternatively, the turbidity of the preparation can be evaluated by simple turbidity measurements well-known to the skilled person. Physical stability of the aqueous protein preparations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

The term “chemical stability” of the protein preparation as used herein refers to changes in the covalent protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Increasing amounts of chemical degradation products is often seen during storage and use of the protein preparation. Most proteins are prone to deamidation, a process in which the side chain amide group in glutaminyl or asparaginyl residues is hydrolysed to form a free carboxylic acid or asparaginyl residues to form an IsoAsp derivative. Other degradations pathways involves formation of high molecular weight products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein preparation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC). Since HMWP products are potentially immunogenic and not biologically active, low levels of HMWP are advantageous.

The term “stabilized preparation” refers to a preparation with increased physical stability, increased chemical stability or increased physical and chemical stability. In general, a preparation must be stable during use and storage (in compliance with recommended use and storage conditions) until the expiration date is reached.

### Pharmaceutically Acceptable Salt, Amide, or Ester

The analogues and intermediate products of the invention may be in the form of a pharmaceutically acceptable salt, amide, or ester.

Salts are e.g. formed by a chemical reaction between a base and an acid, e.g.: 2NH₃+H₂SO₄→(NH₄)₂SO₄.

The salt may be a basic salt, an acid salt, or it may be neither nor (i.e. a neutral salt). Basic salts produce hydroxide ions and acid salts hydronium ions in water.

The salts of the analogues of the invention may be formed with added cations or anions between anionic or cationic groups, respectively. These groups may be situated in the peptide moiety, and/or in the side chain of the analogues of the invention.

Non-limiting examples of anionic groups of the analogues of the invention include free carboxylic groups in the side chain, if any, as well as in the peptide moiety. The peptide moiety often includes a free carboxylic acid group at the C-terminus, and it may also include free carboxylic groups at internal acid amino acid residues such as Asp and Glu.

Non-limiting examples of cationic groups in the peptide moiety include the free amino group at the N-terminus, if present, as well as any free amino group of internal basic amino acid residues such as His, Arg, and Lys.

The ester of the analogues of the invention may, e.g., be formed by the reaction of a free carboxylic acid group with an alcohol or a phenol, which leads to replacement of at least one hydroxyl group by an alkoxy or aryloxy group.

The ester formation may involve the free carboxylic group at the C-terminus of the peptide, and/or any free carboxylic group in the side chain.

The amide of the analogues of the invention may, e.g., be formed by the reaction of a free carboxylic acid group with an amine or a substituted amine, or by reaction of a free or substituted amino group with a carboxylic acid.

The amide formation may involve the free carboxylic group at the C-terminus of the peptide, any free carboxylic group in the side chain, the free amino group at the N-terminus of the peptide, and/or any free or substituted amino group of the peptide in the peptide and/or the side chain.

In a particular embodiment, the peptide is in the form of a pharmaceutically acceptable salt. In another particular embodiment, the peptide is in the form of a pharmaceutically acceptable amide, preferably with an amide group at the C-terminus of the peptide. In a still further particular embodiment, the peptide is in the form a pharmaceutically acceptable ester.

The term “dyslipidemia” as used herein refers to a disorder associated with any altered amount of any or all of the lipids or lipoproteins in the blood. Dyslipidemic disordes include, for example hyperlipidemia, hyperlipoproteinemia, hypercholesterolemia, hypertriglyceridemia, HDL deficiency, apoA-I deficiency, and cardiovascular disease (e.g. coronary artery disease, atherosclerosis and restenosis).

The term “pharmaceutically acceptable” as used herein means suited for normal pharmaceutical applications, i.e. giving rise to no adverse events in patients etc.

The term “excipient” as used herein means the chemical compounds which are normally added to pharmaceutical compositions, e.g. buffers, tonicity agents, preservatives and the like.

The term “effective amount” as used herein means a dosage which is sufficient to be effective for the treatment of the patient compared with no treatment.

The term “pharmaceutical composition” as used herein means a product comprising an active compound or a salt thereof together with pharmaceutical excipients such as buffer, preservative, and optionally a tonicity modifier and/or a stabilizer. Thus a pharmaceutical composition is also known in the art as a pharmaceutical formulation.

The term “treatment of a disease” as used herein means the management and care of a patient having developed the disease, condition or disorder and includes treatment, prevention or alleviation of the disease. The purpose of treatment is to combat the disease, condition or disorder. Treatment includes the administration of the active compounds to eliminate or control the disease, condition or disorder as well as to alleviate the symptoms or complications associated with the disease, condition or disorder, and prevention of the disease, condition or disorder.

The term “diabetes” or “diabetes mellitus” includes type 1 diabetes, type 2 diabetes, gestational diabetes (during pregnancy) and other states that cause hyperglycaemia. The term is used for a metabolic disorder in which the pancreas produces insufficient amounts of insulin, or in which the cells of the body fail to respond appropriately to insulin thus preventing cells from absorbing glucose. As a result, glucose builds up in the blood.

Type 1 diabetes, also called insulin-dependent diabetes mellitus (IDDM) and juvenile-onset diabetes, is caused by beta-cell destruction, usually leading to absolute insulin deficiency.

Type 2 diabetes, also known as non-insulin-dependent diabetes mellitus (NIDDM) and adult-onset diabetes, is associated with predominant insulin resistance and thus relative insulin deficiency and/or a predominantly insulin secretory defect with insulin resistance.

The term “cardiovascular disease” or “CVD” refers to a class of diseases that involve the heart or blood vessels (arteries, capillaries and veins). Cardiovascular disease refers to any disease that affects the cardiovascular system, principally cardiac disease, vascular diseases of the brain and kidney, and peripheral arterial disease. The causes of cardiovascular disease are diverse but atherosclerosis and/or hypertension are the most common. Types of CVD include, coronary heart disease (also ischaemic heart disease or coronary artery disease), cardiomyopathy (diseases of cardiac muscle), hypertensive heart disease (diseases of the heart secondary to high blood pressure), heart failure, coronary heart disease, pulmonale (a failure of the right side of the heart), cardiac dysrhythmias (abnormalities of heart rhythm), inflammatory heart disease (such as endocarditis (inflammation of the inner layer of the heart, the endocardium), inflammatory cardiomegaly and myocarditis (inflammation of the myocardium, the muscular part of the heart), valvular heart disease, stroke and cerebrovascular disease; and peripheral arterial disease.

In one embodiment, the peptides of the present invention, can be used in combination with statins (HMG-CoA reductase inhibitors) e.g. atorvastatin (Lipitor and Torvast), fluvastatin (Lescol), lovastatin (Mevacor, Altocor, Altoprev), pitavastatin (Livalo, Pitava), pravastatin (Pravachol, Selektine, Lipostat), rosuvastatin (Crestor) or simvastatin, or fibrates Bezafibrate (e.g. Bezalip), Ciprofibrate (e.g. Modalim), Gemfibrozil (e.g. Lopid), Fenofibrate (e.g. TriCor) to treat hyperlipidemia, hypertriglyceridemia, hypercholesterolemia, and/or cardiovascular disease such as atherosclerosis.

In another embodiment, the peptides of the present invention, can be used in combination with anti-microbial and/or anti-inflammatory agents such as for example, but not limited to aspirin. The peptides of the present invention can be used in combination with anti-hypertensive medicines known to one of ordinary skill in the art. It is to be understood that more than one additional therapy may be combined with administration of the peptides of the present invention.

In one embodiment, the peptides of the present invention may be administered to an animal or human suffering from a dyslipidemic or vascular disorder, such as hyperlipidemia, hypertriglyceridemia, hypercholesterolemia, hyperlipoproteinemia, HDL deficiency, apoA-I deficiency, coronary artery disease, atherosclerosis, stroke, ischemia, infarction, myocardial infarction, hemorrhage, periferal vascular disease, restenosis, acute coronary syndrome, or reperfusion myocardial injury, in an amount sufficient to inhibit or treat the dyslipidemic or vascular disorder. Amounts effective for this use will depend upon the severity of the disorder and the general state of the subject's health. A therapeutically effective amount of the peptide is that which provides either subjective relief of a symptom(s) or an objective identifiable improvement as noted by the clinician or other qualified observer.

The amino acid abbreviations used in the present context have the following meanings:

| Amino acid | Amino acid | |
|---|---|---|
| (Three-letter | One-letter | |
| code) | code | Description |
| Aib | | 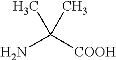 |
| Ala | A | Alanine |
| Asn | N | Asparagine |
| Asp | D | Aspartic acid |
| Arg | R | Arginine |
| Cys | C | Cysteine |
| Gln | Q | Glutamine |
| Glu | E | Glutamic acid |
| Gly | G | Glycine |
| His | H | Histidine |
| desamino- | | Imidazopropionic acid |
| Histidine | | |
| (DesH) | | 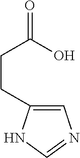 |
| Ile | I | Isoleucine |
| Leu | L | Leucine |
| Lys | K | Lysine |
| Met | M | Methionine |
| Norleucine (Nle) | | 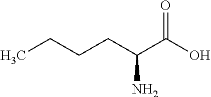 |
| Ornithine (Orn) | | 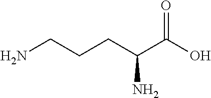 |
| Phe | F | Phenylalanine |
| Pro | P | Proline |
| Ser | S | Serine |
| Thr | T | Threonine |
| Tyr | Y | Tyrosine |
| Trp | W | Tryptophan |
| Val | V | Valine |

### Functional Properties

### Biological Activity—In Vitro Potency

In a particular embodiment, potency and/or activity refers to in vitro potency, i.e. performance in a functional GLP-1 receptor assay, more in particular to the capability of activating the human GLP-1 receptor. The response of the human GLP-1 receptor may be measured in a reporter gene assay, e.g. in a stably transfected BHK cell line that expresses the human GLP-1 receptor and contains the DNA for the cAMP response element (CRE) coupled to a promoter and the gene for firefly luciferase (CRE luciferase). When cAMP is produced as a result of activation of the GLP-1 receptor this in turn results in the luciferase being expressed. Luciferase may be determined by adding luciferin, which by the enzyme is converted to oxyluciferin and produces bioluminescence, which is measured and is a measure of the in vitro potency. One non-limiting example of such an assay is described in Example 2.

The term half maximal effective concentration (EC₅₀) generally refers to the concentration which induces a response halfway between the baseline and maximum, by reference to the dose response curve. EC₅₀ is used as a measure of the potency of a compound and represents the concentration where 50% of its maximal effect is observed.

The in vitro potency of the peptides of the invention may be determined as described above, and the EC₅₀ of the peptide in question determined. The lower the EC₅₀ value, the better the potency.

In a further particular embodiment, the peptide of the invention has an in vitro potency determined using the method of Example 2 corresponding to an EC₅₀ at or below 10000 pM, more preferably below 5000 pM, even more preferably below 1000 pM, or most preferably below 500 pM.

In a particular embodiment, potency and/or activity refers to in vitro potency, i.e. performance in an assay determining cholesterol efflux, more in particular in a cell or tissue based assay measuring the efflux of cholesterol out of the cells. For example cells like mouse monocyte/macrophage cell line, RAW 264.7 or other cells like but not limited to THP-1, BHK cells transfected with the ABCA1 (and/or ABCG1) transporter or other monocyte or macrophage primary cells or cell lines can be used for establishment of a cholesterol efflux assay. For example, cAMP can be used to up-regulate the ABCA1 transporter allowing the measurement of cholesterol efflux mediated specifically by the ABCA1 transporter.

Also, or alternatively, the cholesterol efflux may be measured by incubating the cells with 3H-Cholesterol and subsequently measuring the amount of cholesterol effluxed to the media by measuring the radioactivity of the labelled cholesterol effluxed into the media. Non-specific cholesterol efflux can be measured in non-induced cells (i.e. cells not induced by cAMP). ABCA1-mediated efflux can be obtained from the difference between induced efflux and non-induced efflux. One non-limiting example of such an assay is described in Example 6.

In a further particular embodiment, the peptide of the invention has an in vitro potency determined using the method of Example 6 corresponding to an EC₅₀ potency at or below 2 μM, even more preferably at or below 1 μM, or most preferably below 0.8 μM.

In a further particular embodiment, the peptide of the invention has an in vitro Eₘₐₓ, as determined by the method of Example 6, at or above 65% of the Eₘₐₓ of L-4F, or most preferably at or above 75% of the Eₘₐₓ of L-4F.

The cholesterol efflux potency of the compounds of the present invention of the present invention can also be expressed relative to that of L-4F.

In a further particular, the cholesterol efflux potency of the compounds of the present invention has an EC₅₀ value at or below that of L-4F.

### Biological Activity—In Vivo Pharmacology

In another particular embodiment the peptides of the invention or analogues thereof), are potent in vivo, which may be determined as is known in the art in any suitable animal model, as well as in clinical trials.

The diabetic db/db mouse is one example of a suitable animal model, and the blood glucose lowering effect may be determined in such mice in vivo, e.g. as described in Example 7.

### Cholesterol Efflux

According to the third functional aspect, the peptides of the invention have cholesterol efflux activity. Cholesterol efflux is assessed in vitro by measuring the capacity of compounds to efflux cholesterol from macrophage cell line, primarily transported via the ABCA1 transporter.

The cholesterol efflux activity is determined in vitro as described in Example 6.

ABCA1-mediated efflux may be obtained from the difference between induced efflux and non-induced efflux. EC₅₀ values which were calculated by the software and reported in μM are shown in Table 6, as well as Eₘₐₓ values in %. FIG. 2 show the cholesterol efflux curves for Compound 1, hGLP-1 and Exendin-4.

### Pharmacokinetics Profile

According to the fourth functional aspect, the peptides of the invention have improved pharmacokinetic properties such as increased terminal half-life.

Increased oral bioavailability means that a larger fraction of the dose administered orally reach the systemic circulation from where it can distribute to exhibit pharmacological effect.

The pharmacokinetic properties of the peptides of the invention may suitably be determined in-vivo in pharmacokinetic (PK) studies. Such studies are conducted to evaluate how pharmaceutical compounds are absorbed, distributed, and eliminated in the body, and how these processes affect the concentration of the compound in the body, over the course of time.

In the discovery and preclinical phase of pharmaceutical drug development, animal models such as the mouse, rat, monkey, dog, or pig, may be used to perform this characterisation. Any of these models can be used to test the pharmacokinetic properties of the peptides of the invention.

In such studies, animals are typically administered with a single dose of the drug, either intravenously (i.v.), subcutaneously (s.c.), or orally (p.o.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

For most compounds, the terminal part of the plasma-concentration profiles will be linear when drawn in a semi-logarithmic plot, reflecting that after the initial absorption and distribution, drug is removed from the body at a constant fractional rate. The rate (lambda Z or 2) is equal to minus the slope of the terminal part of the plot. From this rate, also a terminal half-life may be calculated, as T_{1/2}=ln(2)/λ_{z} (see, e.g. Johan Gabrielsson and Daniel Weiner: Pharmacokinetics and Pharmacodynamic Data Analysis. Concepts & Applications, 3rd Ed., Swedish Pharmaceutical Press, Stockholm, 2000).

Clearance can be determined after i.v. administration and is defined as the dose (D) divided by area under the curve (AUC) on the plasma concentration versus time profile (Rowland, M and Tozer T N: Clinical Pharmacokinetics: Concepts and Applications, 3^{rd} edition, 1995 Williams Wilkins).

The estimate of terminal half-life and/or clearance is relevant for evaluation of dosing regimens and an important parameter in drug development, in the evaluation of new drug compounds.

### Pharmacokinetics Profile—Half Life In Vivo in Mice

According to the fourth functional aspect, the peptides of the invention have improved pharmacokinetic properties compared to hGLP-1. Preferably the peptides of the invention have pharmacokinetic properties suitable for once daily administration.

In a particular embodiment, the pharmacokinetic properties may be determined as terminal half-life (T_{1/2}) in vivo in mice after i.v. and s.c. administration. In additional embodiments, the terminal half-life is at least 1 hour, preferably at least 3 hours, preferably at least 4 hours, even more preferably at least 5 hours, or most preferably at least 6 hours.

A suitable assay for determining terminal half-life in vivo in mice after i.v. and s.c. administration is disclosed in Example 8 herein.

### Pharmacokinetics Profile—Half Life In Vivo in Mini-Pigs

According to the fourth functional aspect, the peptides of the invention have improved pharmacokinetic properties compared to hGLP-1 and preferably suitable for once daily administration.

In a particular embodiment, the pharmacokinetic properties may be determined as terminal half-life (T_{1/2}) in vivo in mini-pigs after i.v. administration, e.g. as described in Example 9 herein.

In particular embodiments, the terminal half-life in mini-pigs is at least 5 hours, preferably at least 10 hours, even more preferably at least 15 hours, or most preferably at least 20 hours.

### Biophysical Properties

According to the fifth aspect, the peptides of the invention have good biophysical properties. These properties include but are not limited to physical stability and/or solubility. These and other biophysical properties may be measured using standard methods known in the art of protein chemistry. In a particular embodiment, these properties are improved as compared to native GLP-1 (SEQ ID 1 or SEQ ID 2). Changed oligomeric properties of the peptides may be at least partly responsible for the improved biophysical properties.

Non-limiting examples of assays to investigate biophysical properties are described in Example 3, Example 4, and Example 5.

Additional particular embodiments of the peptides of the invention are described in the sections headed “particular embodiments” and “additional particular embodiments” before the experimental section.

### Methods of Preparation

The GLP-1 receptor agonist peptide of the invention may be obtained by conventional methods for the preparation of peptides and peptide derivatives, and in particular according to the methods described in the working examples.

The GLP-1 moiety of the invention (or fragments thereof), may for instance be produced by classical peptide synthesis, e.g., solid phase peptide synthesis using t-Boc or Fmoc chemistry or other well established techniques, see, e.g., Greene and Wuts, “Protective Groups in Organic Synthesis”, John Wiley & Sons, 1999, Florencio Zaragoza Dörwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000.

Also, or alternatively, they may be produced by recombinant methods, viz. by culturing a host cell containing a DNA sequence encoding the analogue and capable of expressing the peptide in a suitable nutrient medium under conditions permitting the expression of the peptide. Non-limiting examples of host cells suitable for expression of these peptides are: Escherichia coli, Saccharomyces cerevisiae, as well as mammalian BHK or CHO cell lines.

Those peptides of the invention which include non-natural amino acids and/or a covalently attached N-terminal mono- or dipeptide mimetic may e.g. be produced as described in the experimental part, or as described by Hodgson et al: The synthesis of peptides and proteins containing non-natural amino acids; Chemical Society Reviews 2004 33 7 422-430; or as described in WO 2009/083549 A1 entitled “Semi-recombinant preparation of GLP-1 analogues”.

Specific examples of methods of preparing a number of the peptides of the invention are included in the experimental part.

### Pharmaceutical Compositions

Pharmaceutical compositions comprising a peptide of the invention or a pharmaceutically acceptable salt, amide, or ester thereof, and a pharmaceutically acceptable excipient may be prepared as is known in the art.

The term “excipient” broadly refers to any component other than the active therapeutic ingredient(s). The excipient may be an inert substance, an inactive substance, and/or a not medicinally active substance.

The excipient may serve various purposes, e.g. as a carrier, vehicle, diluent, and/or to improve administration, and/or absorption of the active substance.

The formulation of pharmaceutically active ingredients with various excipients is known in the art, see e.g. Remington: The Science and Practice of Pharmacy (e.g. 19^{th} edition (1995), and any later editions).

Non-limiting examples of excipients are: Solvents, diluents, buffers, preservatives, tonicity regulating agents, chelating agents, and stabilisers.

Examples of formulations include liquid formulations, i.e. aqueous formulations comprising water. A liquid formulation may be a solution, or a suspension. An aqueous formulation typically comprises at least 50% w/w water, or at least 60%, 70%, 80%, or even at least 90% w/w of water.

The pH in an aqueous formulation may be anything between pH 3 and pH 10, for example from about 7.0 to about 9.5; or from about 3.0 to about 9.0.

A pharmaceutical composition may comprise a buffer. A pharmaceutical composition may comprise a preservative. A pharmaceutical composition may comprise a chelating agent. The chelating agent may e.g. be selected from salts of ethylenediaminetetraacetic acid (EDTA), citric acid, and aspartic acid, and mixtures thereof.

A pharmaceutical composition may comprise a stabiliser. The stabiliser may e.g. be one or more oxidation inhibitors, aggregation inhibitors, surfactants, and/or one or more protease inhibitors. Non-limiting examples of these various kinds of stabilisers are disclosed in the following.

The term “aggregate formation” refers to a physical interaction between the polypeptide molecules resulting in formation of oligomers, which may remain soluble, or large visible aggregates that precipitate from the solution. Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

A pharmaceutical composition may comprise an amount of an amino acid base sufficient to decrease aggregate formation of the polypeptide during storage of the composition. The term “amino acid base” refers to one or more amino acids (such as methionine, histidine, imidazole, arginine, lysine, isoleucine, aspartic acid, tryptophan, threonine), or analogues thereof. Any amino acid may be present either in its free base form or in its salt form. Any stereoisomer (i.e., L, D, or a mixture thereof) of the amino acid base may be present.

Methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. Any stereoisomer of methionine (L or D) or combinations thereof can be used.

A pharmaceutical composition may comprise a stabiliser selected from the group of high molecular weight polymers or low molecular compounds. A pharmaceutical composition may comprise additional stabilising agents such as, but not limited to, methionine and EDTA, which protect the polypeptide against methionine oxidation, and a nonionic surfactant, which protects the polypeptide against aggregation associated with freeze-thawing or mechanical shearing.

A pharmaceutical composition may comprise one or more surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, nonionic surfactants, and/or zwitterionic surfactants.

A pharmaceutical composition may comprise one or more protease inhibitors.

Additional, optional, ingredients of a pharmaceutical composition include, e.g., wetting agents, emulsifiers, antioxidants, bulking agents, metal ions, oily vehicles, proteins (e.g., human serum albumin, gelatine), and/or a zwitterion (e.g., an amino acid such as betaine, taurine, arginine, glycine, lysine and histidine).

An administered dose may contain from 0.01 mg-100 mg of the peptide or from 0.1-50 mg, or from 1-25 mg of the peptide.

The GLP-1 receptor agonist peptide of the present invention may be administered in the form of a pharmaceutical composition. It may be administered to a patient in need thereof at several sites, for example, at topical sites such as skin or mucosal sites; at sites which bypass absorption such as in an artery, in a vein, or in the heart; and at sites which involve absorption, such as in the skin, under the skin, in a muscle, or in the abdomen.

The route of administration may be, for example, lingual; sublingual; buccal; in the mouth; oral; in the stomach; in the intestine; nasal; pulmonary, such as through the bronchioles, the alveoli, or a combination thereof; parenteral, epidermal; dermal; transdermal; conjunctival; uretal; vaginal; rectal; and/or ocular.

A composition may be administered in several dosage forms, for example as a solution; a suspension; an emulsion; a microemulsion; multiple emulsions; a foam; a salve; a paste; a plaster; an ointment; a tablet; a coated tablet; a chewing gum; a rinse; a capsule such as hard or soft gelatine capsules; a suppositorium; a rectal capsule; drops; a gel; a spray; a powder; an aerosol; an inhalant; eye drops; an ophthalmic ointment; an ophthalmic rinse; a vaginal pessary; a vaginal ring; a vaginal ointment; an injection solution; an in situ transforming solution such as in situ gelling, setting, precipitating, and in situ crystallisation; an infusion solution; or as an implant. A composition may be a tablet, optionally coated, a capsule, or a chewing gum.

A composition may further be compounded in a drug carrier or drug delivery system, e.g. in order to improve stability, bioavailability, and/or solubility. In a particular embodiment a composition may be attached to such system through covalent, hydrophobic, and/or electrostatic interactions. The purpose of such compounding may be, e.g., to decrease adverse effects, achieve chronotherapy, and/or increase patient compliance.

A composition may also be used in the formulation of controlled, sustained, protracting, retarded, and/or slow release drug delivery systems.

Parenteral administration may be performed by subcutaneous, intramuscular, intraperitoneal, or intravenous injection by means of a syringe, optionally a pen-like syringe, or by means of an infusion pump.

A composition may be administered nasally in the form of a solution, a suspension, or a powder; or it may be administered pulmonally in the form of a liquid or powder spray.

Transdermal administration is a still further option, e.g. by needle-free injection, from a patch such as an iontophoretic patch, or via a transmucosal route, e.g. buccally.

A composition may be a stabilised formulation. The term “stabilised formulation” refers to a formulation with increased physical and/or chemical stability, preferably both. In general, a formulation must be stable during use and storage (in compliance with recommended use and storage conditions) until the expiration date is reached.

The term “physical stability” refers to the tendency of the polypeptide to form biologically inactive and/or insoluble aggregates as a result of exposure to thermo-mechanical stress, and/or interaction with destabilising interfaces and surfaces (such as hydrophobic surfaces). The physical stability of an aqueous polypeptide formulation may be evaluated by means of visual inspection, and/or by turbidity measurements after exposure to mechanical/physical stress (e.g. agitation) at different temperatures for various time periods. Alternatively, the physical stability may be evaluated using a spectroscopic agent or probe of the conformational status of the polypeptide such as e.g. Thioflavin T or “hydrophobic patch” probes.

The term “chemical stability” refers to chemical (in particular covalent) changes in the polypeptide structure leading to formation of chemical degradation products potentially having a reduced biological potency, and/or increased immunogenic effect as compared to the intact polypeptide. The chemical stability can be evaluated by measuring the amount of chemical degradation products at various time-points after exposure to different environmental conditions, e.g. by SEC-HPLC, and/or RP-HPLC.

The treatment with a peptide according to the present invention may also be combined with one or more additional pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity.

Examples of these pharmacologically active substances are: Insulins and insulin analogues such as but not limited to Lantus also known as insulin glargine, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, serotonin re-uptake inhibitors, serotonin and noradrenaline re-uptake inhibitors, mixed serotonin and noradrenergic compounds, 5HT (serotonin) agonists, galanin antagonists, growth hormone, growth hormone releasing compounds, TRH (thyreotropin releasing hormone) agonists, UCP 2 or 3 (uncoupling protein 2 or 3) modulators, DA agonists (bromocriptin, doprexin), lipase/amylase inhibitors, RXR (retinoid X receptor) modulators, TR β agonists; histamine H3 antagonists. The treatment with a peptide according to this invention may also be combined with a surgery that influences the glucose levels, and/or lipid homeostasis such as gastric banding or gastric bypass.

### Medical Indications

The present invention also relates to peptides for use as a medicaments.

In particular embodiments, the peptides of the invention may be used for the following medical treatments, all preferably relating one way or the other to diabetes or cardiovascular disease or the combination of the two:

(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;

(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;

(iii) improving β-cell function, such as decreasing β-cell apoptosis, increasing β-cell function and/or β-cell mass, and/or for restoring glucose sensitivity to β-cells;

(iv) prevention and/or treatment of cognitive disorders;

(v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delaying gastric emptying;

(vi) prevention and/or treatment of diabetic complications, such as neuropathy, including peripheral neuropathy; nephropathy; or retinopathy;

(vii) improving lipid parameters, such as prevention and/or treatment of dyslipidemia, lowering total serum lipids; lowering HDL; lowering small, dense LDL; lowering VLDL: lowering triglycerides; lowering cholesterol; increasing HDL; lowering plasma levels of lipoprotein a (Lp(a)) in a human; inhibiting generation of apolipoprotein a (apo(a));

(iix) prevention and/or treatment of cardiovascular diseases, such as but not limited to hyperlipidemia, hyperlipoproteinemia, hypercholesterolemia, hypertriglyceridemia, HDL deficiency, apoA-I deficiency, coronary heart disease, atherosclerosis, thrombotic stroke, stroke, peripheral vascular disease, restenosis, acute coronary syndrome, reperfusion myocardial injury, syndrome X; myocardial infarction; cerebral ischemia; an early cardiac or early cardiovascular disease, such as left ventricular hypertrophy; coronary artery disease; essential hypertension; acute hypertensive emergency; cardiomyopathy; heart insufficiency; exercise tolerance; chronic heart failure; arrhythmia; cardiac dysrhythmia; syncopy; atheroschlerosis; mild chronic heart failure; angina pectoris; cardiac bypass reocclusion; intermittent claudication (atheroschlerosis oblitterens); diastolic dysfunction; and/or systolic dysfunction;

(ix) prevention and/or treatment of gastrointestinal diseases, such as inflammatory bowel syndrome; small bowel syndrome, or Crohn's disease; dyspepsia; and/or gastric ulcers;

(x) prevention and/or treatment of critical illness, such as treatment of a critically ill patient, a critical illness poly-nephropathy (CIPNP) patient, and/or a potential CIPNP patient; prevention of critical illness or development of CIPNP; prevention, treatment and/or cure of systemic inflammatory response syndrome (SIRS) in a patient; and/or for the prevention or reduction of the likelihood of a patient suffering from bacteraemia, septicaemia, and/or septic shock during hospitalisation; and/or

(xi) prevention and/or treatment of polycystic ovary syndrome (PCOS).

In a particular embodiment, the indication is selected from the group consisting of (i)-(iii) and (v)-(iix), such as indications (i), (ii), and/or (iii); or indication (v), indication (vi), indication (vii), and/or indication (iix).

In another particular embodiment, the indication is (i). In a further particular embodiment the indication is (v). In a still further particular embodiment the indication is (iix).

The following indications are particularly preferred: Type 2 diabetes, and/or obesity and/or cardiovascular disease, especially atherosclerosis.

### EMBODIMENTS

The present invention may be further defined by reference to the following

### EMBODIMENTS

1. A GLP-1 receptor agonist peptide which in an alpha helical conformation comprises an amphipathic helix, wherein said peptide has cholesterol efflux activity.

2. A GLP-1 receptor agonist peptide which in an alpha helical conformation comprise an amphipathic helix, wherein said peptide has cholesterol efflux activity with an Eₘₐₓ of at least 65% of that of L-4F, and a potency measured as EC₅₀, that is better than the potency of L-4F, when measured according to the methods described in Example 6.

3. The GLP-1 receptor agonist peptide of embodiment 2, wherein said peptide comprises at least 31 amino acid residues.

4. The GLP-1 receptor agonist peptide of embodiment 2, wherein said peptide comprises at least 32 amino acid residues.

5. The GLP-1 receptor agonist peptide of embodiment 2, wherein said peptide comprises at least 32 amino acid residues.

6. The GLP-1 receptor agonist peptide of embodiment 2, wherein said peptide comprises at least 33 amino acid residues.

7. The GLP-1 receptor agonist peptide of embodiment 2, wherein said peptide comprises at least 34 amino acid residues.

8. The GLP-1 receptor agonist peptide of any one of embodiments 1-7, wherein said amphipathic helix comprises at least 15 amino acid residues.

9. The GLP-1 receptor agonist peptide of any one of embodiments 1-7, wherein said amphipathic helix comprises at least 16 amino acid residues.

10. The GLP-1 receptor agonist peptide of any one of embodiments 1-7, wherein said amphipathic helix comprises at least 17 amino acid residues.

11. The GLP-1 receptor agonist peptide of any one of embodiments 1-7, wherein said amphipathic helix comprises at least 18 amino acid residues.

12. The GLP-1 receptor agonist peptide of any one of the previous embodiments, wherein said amphipathic helix comprises a hydrophilic and a lipophilic face.

13. The GLP-1 receptor agonist peptide of embodiment 12, wherein said hydrophilic face comprises at least six amino acid residues, wherein at least four amino acid residues are charged.

14. The GLP-1 receptor agonist peptide of embodiment 12, wherein said hydrophilic face comprises at least six amino acid residues, wherein at least five amino acid residues are charged.

15. The GLP-1 receptor agonist peptide of embodiment 12, wherein said hydrophilic face comprises at least six amino acid residues, wherein at least six amino acid residues are charged.

16. The GLP-1 receptor agonist peptide any one of embodiments 13-15, wherein said charged amino acids residues comprise at least two negatively charged amino acids and at least 2 positively charged amino acids.

17. The GLP-1 receptor agonist peptide any one of embodiments 13-15, wherein said lipophilic face comprises at least seven amino acid residues, wherein at least six amino acid residues are lipophilic.

18. The GLP-1 receptor agonist peptide any one of embodiments 13-15, wherein said lipophilic face comprises at least seven amino acid residues, wherein at least seven amino acid residues are lipophilic.

19. The GLP-1 receptor agonist peptide any one of embodiments 13-15, wherein said lipophilic face comprises at least eight amino acid residues, wherein at least seven amino acid residues are lipophilic.

20. The GLP-1 receptor agonist peptide any one of embodiments 13-15, wherein said lipophilic face comprises at least eight amino acid residues, wherein at least eight amino acid residues are lipophilic.

21. A GLP-1 receptor agonist peptide of any one of embodiments 1-20, which is a GLP-1 receptor agonist peptide according to any one of embodiments 22-37.

22. A GLP-1 receptor agonist peptide of any one of embodiments 1-21, comprising an amino acid sequence of Formula I:

X₇-X₈-X₉-Gly-Thr-X₁₂-Thr-X₁₄-Asp-X₁₆-X₁₇-X₁₈-X₁₉-X₂₀-X₂₁-X₂₂-X₂₃-X₂₄-X₂₅-X₂₆-X₂₇-Phe-X₂₉-X₃₀-X₃₁-Leu-X₃₃-X₃₄-X₃₆-X₃₆-X₃₇-X₃₈-X₃₉-X₄₀-X₄₁-X₄₂-X₄₃-X₄₄-X₄₅-X₄₆-X₄₇-X₄₈-X₄₉-X₅₀

wherein,

X₇ represents His, or desamino-His;

X₈ represents Ala, Gly, Ser, or Aib;

X₉ represents Glu, Asp, Gln, or His;

X₁₂ represents Phe, Tyr, or Leu;

X₁₄ represents Ser, Asn, or His;

X₁₆ represents Val, Tyr, Leu, Ile, or Met;

X₁₇ represents Ser, or Thr;

X₁₈ represents Ser, Lys, Arg, Glu, Asn, or Gln;

X₁₉ represents Tyr, or Gln;

X₂₀ represents Leu, Met, or Tyr;

X₂₁ represents Glu, Asp, or Gln;

X₂₂ represents Gly, Ser, Glu, Lys, Aib, or Pro;

X₂₃ represents Gln, Glu, Lys, Trp, Arg, or Asp;

X₂₄ represents Ala, Aib, Lys, or Arg;

X₂₅ represents Ala, Val, Phe, His, Leu, Met, Trp, Tyr, Ile, or Aib;

X₂₆ represents Lys, Asn, Glu, Arg, His, Gly, Val, or Gln;

X₂₇ represents Glu, Asp, Gln, Ala, His, Gly, Arg, Lys, Aib, or Leu;

X₂₉ represents Ile, or Val;

X₃₀ represents Ala, Val, Gln, Ile, Trp, Aib, Glu, Arg, or Lys;

X₃₁ represents Trp, Gln, Lys, or His;

X₃₃ represents Val, Met, Ile, Leu, Thr, Arg, or Lys;

X₃₄ represents Lys, Glu, Asn, Asp, Gln, His, Gly, or Arg;

X₃₅ represents Gly, Lys, Arg, His, Ser, Thr, Aib, Ala, or Gln;

X₃₆ represents Gly, Aib, Val, Leu, Ala, His, Ile, Met, Trp, Tyr, or Phe;

X₃₇ represents Gly, Ala, Glu, Aib, His, Arg, Leu, Pro, Lys, or Gln;

X₃₈ represents Glu, Ser, Asp, His, Gly, Gln, or amide, or X₃₈ is absent;

X₃₉ represents Phe, Leu, His, Ala, Ser, Ile, Met, Val, Trp, Tyr, Gly, Glu, Lys, or amide, or X₃₉ is absent;

X₄₀ represents Gly, Leu, Phe, Val, His, Gly, Ala, Ile, Met, Trp, Tyr, or amide, or X₄₀ is absent;

X₄₁ represents Glu, Asp, Ala, Gly, Lys, or amide, or X₄₁ is absent;

X₄₂ represents Leu, Pro, Lys, Arg, or amide, or X₄₂ is absent;

X₄₃ represents Leu, Pro, Val, or amide, or X₄₃ is absent;

X₄₄ represents Lys, or amide, or X₄₄ is absent;

X₄₅ represents Glu, or amide, or X₄₅ is absent;

X₄₆ represents Phe, Ile, or amide, or X₄₆ is absent;

X₄₇ represents Ile, or amide, or X₄₇ is absent;

X₄₈ represents Ala, or amide, or X₄₈ is absent;

X₄₉ represents Trp, or amide, or X₄₉ is absent;

X₅₀ represents amide, or X₅₀ is absent;

with the proviso that if X₃₈, X₃₉, X₄₀, X₄₁, X₄₂, X₄₃, X₄₄, X₄₅, X₄₆, X₄₇, X₄₈, X₄₉ or X₅₀ is absent, then each amino acid residue downstream is also absent;

or a pharmaceutically acceptable salt, amide, ester, or acid, or a prodrug thereof.

23. A GLP-1 receptor agonist peptide of any one of embodiments 1-21, comprising an amino acid sequence of Formula I, as defined above, wherein:

X₇ represents His, or desamino-His;

X₈ represents Ala, Gly, Ser, or Aib;

X₉ represents Glu, Asp, Gln, or His;

X₁₂ represents Phe, Tyr, or Leu;

X₁₄ represents Ser, Asn, or His;

X₁₆ represents Val, Tyr, Leu, Ile, or Met;

X₁₇ represents Ser, or Thr;

X₁₈ represents Ser, Lys, Glu, or Asn;

X₁₉ represents Tyr, or Gln;

X₂₀ represents Leu, Met, or Tyr;

X₂₁ represents Glu, or Asp;

X₂₂ represents Gly, Ser, Glu, or Pro;

X₂₃ represents Gln, Glu, Lys, Trp, Arg, or Asp;

X₂₄ represents Ala, Aib, Lys, or Arg;

X₂₅ represents Ala, or Val;

X₂₆ represents Lys, Arg, or Gln;

X₂₇ represents Glu, Asp, Gln, Lys, or Leu;

X₂₉ represents Ile, or Val;

X₃₀ represents Ala, Val, Gln, Trp, Aib, Glu, or Lys;

X₃₁ represents Trp, Lys, or His;

X₃₃ represents Val, Met, Leu, or Lys;

X₃₄ represents Lys, Glu, Asn, Gln, or His;

X₃₅ represents Gly, Lys, Arg, His, Thr, Ala, or Gln;

X₃₆ represents Gly, Aib, Val, Leu, or Phe;

X₃₇ represents Gly, Ala, His, Arg, Leu, Pro, Lys, or Gln;

X₃₈ represents Glu, Ser, Asp, His, Gly, Gln, or amide, or X₃₈ is absent;

X₃₉ represents Phe, Leu, His, Ala, Val, Trp, Gly, Glu, Lys, or amide, or X₃₉ is absent;

X₄₀ represents Leu, Phe, Val, His, Tyr, or amide, or X₄₀ is absent;

X₄₁ represents Glu, Ala, Asp, Gly, Lys, or amide, or X₄₁ is absent;

X₄₂ represents Leu, Lys, Arg, or amide, or X₄₂ is absent;

X₄₃ represents Leu, Val, or amide, or X₄₃ is absent;

X₄₄ represents Lys, or amide, or X₄₄ is absent;

X₄₅ represents Glu, or X₄₅ is absent;

X₄₆ represents Phe, or X₄₆ is absent;

X₄₇ represents amide, or X₄₇ is absent;

X₄₈ is absent;

X₄₉ is absent;

X₅₀ is absent;

with the proviso that if X₃₈, X₃₉, X₄₀, X₄₁, X₄₂, X₄₃, X₄₄, X₄₅, X₄₆, X₄₇, X₄₈, X₄₉ or X₅₀ is absent, then each amino acid residue downstream is also absent;

or a pharmaceutically acceptable salt, amide, ester, or acid, or a prodrug thereof.

24. A GLP-1 receptor agonist peptide of any one of embodiments 1-21, comprising an amino acid sequence of Formula I, as defined above, wherein:

X₇ represents His;

X₈ represents Ser, or Aib;

X₉ represents Glu, Asp, Gln, or His;

X₁₂ represents Phe, Tyr, or Leu;

X₁₄ represents Ser, Asn, or His;

X₁₆ represents Val, Tyr, Leu, Ile, or Met;

X₁₇ represents Ser, or Thr;

X₁₈ represents Ser, Lys, Glu, or Asn;

X₁₉ represents Tyr, or Gln;

X₂₀ represents Leu, Met, or Tyr;

X₂₁ represents Glu, or Asp;

X₂₂ represents Gly, Ser, or Glu;

X₂₃ represents Gln, Glu, Lys, Arg, or Asp;

X₂₄ represents Ala, Aib, Lys, or Arg;

X₂₅ represents Val;

X₂₆ represents Lys, or Arg;

X₂₇ represents Glu, Asp, or Lys;

X₂₉ represents Ile;

X₃₀ represents Ala, Trp, Aib, or Glu;

X₃₁ represents Trp, Lys, or His;

X₃₃ represents Val, Met, Leu, or Lys;

X₃₄ represents Lys, or Glu;

X₃₅ represents Gly, Lys, Arg, or Thr;

X₃₆ represents Gly, Aib, Leu, or Phe;

X₃₇ represents Gly, Arg, Leu, Pro, or Lys;

X₃₈ represents Glu, or amide, or X₃₈ is absent;

X₃₉ represents Phe, Leu, His, or Ala, or X₃₉ is absent;

X₄₀ represents Leu, Phe, Val, or His, or X₄₀ is absent;

X₄₁ represents Glu, or amide, or X₄₁ is absent;

X₄₂ represents Leu, or Lys, or X₄₂ is absent;

X₄₃ represents Leu, or Val, or X₄₃ is absent;

X₄₄ represents Lys, or amide, or X₄₄ is absent;

X₄₅ represents Glu, or X₄₅ is absent;

X₄₆ represents Phe, or X₄₆ is absent;

X₄₇ represents amide, or X₄₇ is absent;

X₄₈ is absent;

X₄₉ is absent;

X₅₀ is absent;

with the proviso that if X₃₈, X₃₉, X₄₀, X₄₁, X₄₂, X₄₃, X₄₄, X₄₅, X₄₆, X₄₇, X₄₈, X₄₉ or X₅₀ is absent, then each amino acid residue downstream is also absent;

or a pharmaceutically acceptable salt, amide, ester, or acid, or a prodrug thereof.

25. A GLP-1 receptor agonists comprising an amino acid sequence of Formula I, as defined above, wherein X₇-X₃₅ represents Exendin-4 (1-29), GLP-1(7-35) or glucagon peptide (1-29), with up to 12 amino acid substitutions.

26. A GLP-1 receptor agonists comprising an amino acid sequence of Formula I, as defined above, wherein X₇-X₃₅ represents Exendin-4 (1-29), GLP-1(7-35) or glucagon peptide (1-29), with up to 10 amino acid substitutions.

27. The GLP-1 receptor agonist peptide of embodiment 26, wherein X₇-X₃₅ represents Exendin-4 (1-29) with up to 10 amino acid substitutions.

28. The GLP-1 receptor agonist peptide of embodiment 26, wherein X₇-X₃₅ represents Exendin-4 (1-29) with up to 5 amino acid substitutions.

29. The GLP-1 receptor agonist peptide of embodiment 26, wherein X₇-X₃₅ represents GLP-1(7-35) with up to 12 amino acid substitutions.

30. The GLP-1 receptor agonist peptide of embodiment 26, wherein X₇-X₃₅ represents GLP-1(7-35) with up to 10 amino acid substitutions.

31. The GLP-1 receptor agonist peptide of embodiment 26, wherein X₇-X₃₅ represents GLP-1(7-35) with up to 9 amino acid substitutions.

32. The GLP-1 receptor agonist peptide of embodiment 26, wherein X₇-X₃₅ represents glucagon peptide (1-29) with up to 10 amino acid substitutions.

33. The GLP-1 receptor agonist peptide of any one of the previous embodiments, comprising a peptide with a sequence identity of more than 60%, more than 70%, more than 80%, more than 90% or more than 95% to SEQ ID 9, SEQ ID 10 or SEQ ID 11 and with up to 3 additional Aib substitutions.

34. The GLP-1 receptor agonist peptide of any one of the previous embodiments, comprising a peptide with a sequence identity of more than 60%, more than 70%, more than 80%, more than 90% or more than 95% to SEQ ID 9 and with up to 3 additional Aib substitutions.

35. The GLP-1 receptor agonist peptide of any one of the previous embodiments, comprising a peptide with a sequence identity of more than 60%, more than 70%, more than 80%, more than 90% or more than 95% to SEQ ID 10 and with up to 3 additional Aib substitutions.

36. The GLP-1 receptor agonist peptide of any one of the previous embodiments, comprising a peptide with a sequence identity of more than 60%, more than 70%, more than 80%, more than 90% or more than 95% to SEQ ID 11 and with up to 3 additional Aib substitutions.

37. A GLP-1 receptor agonist peptide of any one of embodiments 1-36, comprising an amino acid sequence of Formula I, as defined above, wherein:

X₇ represents His, or desamino-His;

X₈ represents Ala, Gly, Ser, or Aib;

X₉ represents Glu, Asp, Gln, or His;

X₁₂ represents Phe, Tyr, or Leu;

X₁₄ represents Ser, Asn, or His;

X₁₆ represents Val, Tyr, Leu, Ile, or Met;

X₁₇ represents Ser, or Thr;

X₁₈ represents Ser, Lys, Arg, Glu, Asn, or Gln;

X₁₉ represents Tyr, or Gln;

X₂₀ represents Leu, Met, Tyr, or Lys;

X₂₁ represents Glu, Asp, or Gln;

X₂₂ represents Gly, Ser, Glu, Lys, or Aib;

X₂₃ represents Gln, Glu, Lys, Trp, Arg, or Asp;

X₂₄ represents Ala, Aib, Lys, or Arg;

X₂₅ represents Ala, Val, Phe, His, Leu, Met, Trp, Tyr, Ile, or Aib;

X₂₆ represents Lys, Asn, Glu, Arg, His, Gly, or Val;

X₂₇ represents Glu, Asp, Gln, Ala, His, Gly, Arg, Lys, Aib, or Leu;

X₂₉ represents Ile, or Val;

X₃₀ represents Ala, Val, Gln, Ile, Trp, Aib, Glu, or Arg;

X₃₁ represents Trp, Gln, Lys, or His;

X₃₃ represents Val, Met, Ile, Leu, Thr, Arg, or Lys;

X₃₄ represents Lys, Glu, Asn, Asp, Gln, His, Gly or Arg;

X₃₅ represents Gly, Lys, Arg, His, Ser, Thr or Aib;

X₃₆ represents Gly, Aib, Val, Leu, Ala, His, Ile, Met, Trp, Tyr, Phe;

X₃₇ represents Gly, Ala, Glu, Aib, His, Arg, Leu, Pro, or Lys;

X₃₈ represents Glu, Ser, Asp, His, or amide, or X₃₈ is absent;

X₃₉ represents Phe, Leu, His, Ala, Ser, Ile, Met, Val, Trp, Tyr, or amide, or X₃₉ is absent;

X₄₀ represents Leu, Phe, Val, His, Gly, Ala, Ile, Met, Trp, Tyr, or amide, or X₄₀ is absent;

X₄₁ represents Glu, Ala, or amide, or X₄₁ is absent;

X₄₂ represents Leu, Pro, Lys, or amide, X₄₂ or is absent;

X₄₃ represents Leu, Pro, Val, or amide, or X₄₃ is absent;

X₄₄ represents Lys, or amide, or X₄₄ is absent;

X₄₅ represents Glu, or amide, or X₄₅ is absent;

X₄₆ represents Phe, Ile, or amide, or X₄₆ is absent;

X₄₇ represents Ile, or amide, or X₄₇ is absent;

X₄₈ represents Ala, or amide, or X₄₈ is absent;

X₄₉ represents Trp, or amide, or X₄₉ is absent; and

X₅₀ represents amide, or X₅₀ is absent;

with the proviso that if X₃₈, X₃₉, X₄₀, X₄₁, X₄₂, X₄₃, X₄₄, X₄₅, X₄₆, X₄₇, X₄₈, X₄₉ or X₅₀ is absent, then each amino acid residue downstream is also absent;

or a pharmaceutically acceptable salt, amide, ester, or acid, or a prodrug thereof.

38. A GLP-1 receptor agonist peptide comprising an amino acid sequence of Formula I:

X₇-X₈-X₉-Gly-Thr-X₁₂-Thr-X₁₄-Asp-X₁₆-X₁₇-X₁₈-X₁₉-X₂₀-X₂₁-X₂₂-X₂₃-X₂₄-X₂₅-X₂₆-X₂₇-Phe-X₂₉-X₃₀-X₃₁-Leu-X₃₃-X₃₄-X₃₅-X₃₆-X₃₇-X₃₈-X₃₉-X₄₀-X₄₁-X₄₂-X₄₃-X₄₄-X₄₅-X₄₆-X₄₇-X₄₈-X₄₉-X₅₀

wherein,

X₇ represents His, or desamino-His;

X₈ represents Ala, Gly, Ser, or Aib;

X₉ represents Glu, Asp, Gln, or His;

X₁₂ represents Phe, Tyr, or Leu;

X₁₄ represents Ser, Asn, or His;

X₁₆ represents Val, Tyr, Leu, Ile, or Met;

X₁₇ represents Ser, or Thr;

X₁₈ represents Ser, Lys, Arg, Glu, Asn, or Gln;

X₁₉ represents Tyr, or Gln;

X₂₀ represents Leu, Met, or Tyr;

X₂₁ represents Glu, Asp, or Gln;

X₂₂ represents Gly, Ser, Glu, Pro, Lys, or Aib;

X₂₃ represents Gln, Glu, Lys, Trp, or Asp;

X₂₄ represents Ala, Aib, Lys, or Arg;

X₂₅ represents Ala, Val, Leu, Ile, or Aib;

X₂₆ represents Lys, Asn, Glu, Arg, His, Gly, Val, or Gln;

X₂₇ represents Glu, Asp, Gln, Ala, His, Gly, Arg, Lys, Aib, or Leu;

X₂₉ represents Ile, or Val;

X₃₀ represents Ala, Val, Gln, Ile, Trp, Aib, Glu, Arg, or Lys;

X₃₁ represents Trp, Gln, Lys, or His;

X₃₃ represents Val, Ile, Leu, Thr, Arg, or Lys;

X₃₄-X₃₅-X₃₆-X₃₇-X₃₈-X₃₉ represents Subsequence 1, composed by the following amino acid residues “Glu-Lys-Aib-Lys-Glu-Phe”; or in which Subsequence 1, one, two or three amino acid residues have been substituted for

[Asn, Gln, Lys, His, Gly, Arg, or Asp] in position X₃₄;

[Arg, Ala, His, Gln, Gly, Asn, or Aib] in position X₃₅;

[Gly, Val, Leu, Phe, Ile, Trp, Tyr, Ala, Met, or His] in position X₃₆;

[Arg, Ala, Leu, Gly, His, Gln, Asn, Aib, Ile, Val, or Phe] in position X₃₇;

[Asp, His, Gln, Ser, Gly, Asn, or Thr] in position X₃₈; and/or

[Trp, Ala, Glu, Leu, Val, Gly, His, Lys, Ser, Thr, Tyr, Aib, Ile, or Met] in position X₃₉; and

X₄₀ represents Leu, Phe, Val, His, Tyr, or amide, or X₄₀ is absent;

X₄₁ represents Glu, Asp, Ala, Gly, Lys, or amide, or X₄₁ is absent;

X₄₂ represents Leu, Pro, Lys, Arg, or amide, or X₄₂ is absent;

X₄₃ represents Leu, Pro, Val, or amide, or X₄₃ is absent;

X₄₄ represents Lys, or amide, or X₄₄ is absent;

X₄₅ represents Glu, or amide, or X₄₅ is absent;

X₄₆ represents Phe, Ile, or amide, or X₄₆ is absent;

X₄₇ represents Ile, or amide, or X₄₇ is absent;

X₄₈ represents Ala, or amide, or X₄₈ is absent;

X₄₉ represents Trp, or amide, or X₄₉ is absent;

X₅₀ represents amide, or X₅₀ is absent;

provided, however:

if X₄₁, X₄₂, X₄₃, X₄₄, X₄₅, X₄₆, X₄₇, X₄₈, X₄₉ or X₅₀ is absent, then each amino acid residue downstream is also absent;

and pharmaceutically acceptable salts, amides, esters, acids or prodrugs thereof.

39. The GLP-1 receptor agonist peptide of embodiment 38, wherein in Subsequence 1, one amino acid residue has been substituted.

40. The GLP-1 receptor agonist peptide of embodiment 38, wherein in Subsequence 1, two amino acid residues have been substituted.

41. The GLP-1 receptor agonist peptide of embodiment 38, wherein in Subsequence 1, three amino acid residues have been substituted.

42. The GLP-1 receptor agonist peptide of any one of embodiments 38-41, wherein,

Glu of position X₃₄ has been changed for Asn, Gln, Lys, His, or Gly; and/or

Lys of position X₃₅ has been changed for Arg, Ala, His, or Gln; and/or

Aib of position X₃₆ has been changed for Gly, Val, Leu, or Phe; and/or

Lys of position X₃₇ has been changed for Arg, Ala, Leu, Gly, His, or Gln; and/or

Glu of position X₃₈ has been changed for Asp, His, Gln, Ser, or Gly; and/or

Phe of position X₃₉ has been changed for Trp, Ala, Glu, Leu, Val, Gly, His, Lys, or Ser.

43. The GLP-1 receptor agonist peptide of embodiment 42, wherein,

Glu of position X₃₄ has been changed for Asn, Gln, Lys, or His; and/or

Lys of position X₃₅ has been changed for Arg, Ala, His, or Gln; and/or

Aib of position X₃₆ has been changed for Gly, Val, Leu, or Phe; and/or

Lys of position X₃₇ has been changed for Arg, Ala, Leu, Gly, His, or Gln; and/or

Glu of position X₃₈ has been changed for Asp, His, Gln, Ser, or Gly; and/or

Phe of position X₃₉ has been changed for Trp, Ala, Glu, Leu, Val, Gly, His, or Lys.

44. The GLP-1 receptor agonist peptide of embodiment 41, wherein,

Glu of position X₃₄ has been changed for Lys, or Gly; and/or

Lys of position X₃₅ has been changed for Arg; and/or

Aib of position X₃₆ has been changed for Gly, Leu, or Phe; and/or

Lys of position X₃₇ has been changed for Arg, Leu, or Gly; and/or

Glu of position X₃₈ remains unchanged; and/or

Phe of position X₃₉ has been changed for Ala, Leu, His, or Ser.

45. The GLP-1 receptor agonist peptide of embodiment 38, wherein X₃₄-X₃₅-X₃₆-X₃₇-X₃₈-X₃₉-X₄₀ represents Subsequence 2, composed of the following amino acid residues “Glu-Lys-Aib-Lys-Glu-Phe-Leu”; or in which Subsequence 2, one, two or three amino acid residues have been substituted for:

[Asn, Gln, Lys, His, Gly, Arg, or Asp] in position X₃₄;

[Arg, Ala, His, Gln, Gly, Asn, or Aib] in position X₃₅;

[Gly, Val, Leu, Phe, Ile, Trp, Tyr, Ala, Met, or His] in position X₃₆;

[Arg, Ala, Leu, Gly, His, Gln, Asn, Aib, Ile, Val, or Phe] in position X₃₇;

[Asp, His, Gln, Ser, Gly, Asn, or Thr] in position X₃₈;

[Trp, Ala, Glu, Leu, Val, Gly, His, Lys, Ser, Thr, Tyr, Aib, Ile, or Met] in position X₃₉; and/or

[Phe, Gly, Val, Tyr, His, Ile, Trp, Ala, Aib, or Met] in position X₄₀.

46. The GLP-1 receptor agonist peptide of embodiment 45, wherein in Subsequence 2, one amino acid residue has been substituted.

47. The GLP-1 receptor agonist peptide of embodiment 45, wherein in Subsequence 2, two amino acid residues have been substituted.

48. The GLP-1 receptor agonist peptide of embodiment 45, wherein in Subsequence 2, three amino acid residues have been substituted.

49. The GLP-1 receptor agonist peptide of any one of embodiments 46-48, wherein,

Glu of position X₃₄ has been changed for Asn, Gln, Lys, His, or Gly; and/or

Lys of position X₃₅ has been changed for Arg, Ala, His, or Gln; and/or

Aib of position X₃₆ has been changed for Gly, Val, Leu, or Phe; and/or

Lys of position X₃₇ has been changed for Arg, Ala, Leu, Gly, His, or Gln; and/or

Glu of position X₃₈ has been changed for Asp, His, Gln, Ser, or Gly; and/or

Phe of position X₃₉ has been changed for Trp, Ala, Glu, Leu, Val, Gly, His, Lys, or Ser; and/or

Leu of possition X₄₀ has been changed for Phe, Gly, Val, Tyr, or His.

50. The GLP-1 receptor agonist peptide of embodiment 49, wherein,

Glu of position X₃₄ has been changed for Lys; and/or

Lys of position X₃₅ has been changed for Arg; and/or

Aib of position X₃₆ has been changed for Gly, Leu, or Phe; and/or

Lys of position X₃₇ has been changed for Arg, Leu, or Gly; and/or

Glu of position X₃₈ remains unchanged; and/or

Phe of position X₃₉ has been changed for Ala, Leu, or His; and/or

Leu of possition X₄₀ has been changed for Phe, Val, or His.

51. A GLP-1 receptor agonists comprising an amino acid sequence of Formula I, as defined above, wherein,

X₃₈ is Glu, Ser, Asp or His;

X₃₉ is Phe, Leu, His, Ala, Ser, Ile, Met, Val, Trp or Tyr; and

X₄₀ is Leu, Phe, Val, His, Gly, Ala, Ile, Met, Trp or Tyr.

52. A GLP-1 receptor agonists comprising an amino acid sequence of Formula I, as defined above, wherein X₇ represents His or desamino-His.

53. A GLP-1 receptor agonists comprising an amino acid sequence of Formula I, as defined above, wherein X₈ represents Ala, Gly, Ser, or Aib.

54. A GLP-1 receptor agonists comprising an amino acid sequence of Formula I, as defined above, wherein X₈ is Ala, Gly, Ser or Aib.

55. A GLP-1 receptor agonists comprising an amino acid sequence of Formula I, as defined above, wherein X₉ is Glu, Asp, Gln or His.

56. A GLP-1 receptor agonists comprising an amino acid sequence of Formula I, as defined above, wherein X₉ represents Glu, Asp, or Gln.

57. A GLP-1 receptor agonists comprising an amino acid sequence of Formula I, as defined above, wherein X₁₂ represents Phe, Tyr, or Leu.

58. A GLP-1 receptor agonists comprising an amino acid sequence of Formula I, as defined above, wherein X₁₄ represents Ser, Asn, or His.

59. A GLP-1 receptor agonists comprising an amino acid sequence of Formula I, as defined above, wherein X₁₆ represents Val, Tyr, Leu, Ile, or Met.

60. A GLP-1 receptor agonists comprising an amino acid sequence of Formula I, as defined above, wherein X₁₇ represents Ser, or Thr.

61. A GLP-1 receptor agonists comprising an amino acid sequence of Formula I, as defined above, wherein X₁₈ is Ser, Lys, Arg, Glu, Asn or Gln.

62. A GLP-1 receptor agonists comprising an amino acid sequence of Formula I, as defined above, wherein X₁₈ represents Ser, Lys, Glu, or Asn.

63. A GLP-1 receptor agonists comprising an amino acid sequence of Formula I, as defined above, wherein X₁₉ represents Tyr, or Gln.

64. A GLP-1 receptor agonists comprising an amino acid sequence of Formula I, as defined above, wherein X₂₀ represents Leu, Met, Tyr, or Lys.

65. A GLP-1 receptor agonists comprising an amino acid sequence of Formula I, as defined above, wherein X₂₀ represents Leu, Met, or Tyr.

66. A GLP-1 receptor agonists comprising an amino acid sequence of Formula I, as defined above, wherein X₂₁ is Glu, Asp, or Gln.

67. A GLP-1 receptor agonists comprising an amino acid sequence of Formula I, as defined above, wherein X₂₁ represents Glu, or Asp.

68. A GLP-1 receptor agonists comprising an amino acid sequence of Formula I, as defined above, wherein X₂₂ represents Gly, Ser, Glu, Pro, Lys, or Aib.

69. A GLP-1 receptor agonists comprising an amino acid sequence of Formula I, as defined above, wherein X₂₂ represents Gly, Ser, Glu, or Pro.

70. A GLP-1 receptor agonists comprising an amino acid sequence of Formula I, as defined above, wherein X₂₂ is Gly, Ser, Glu, Lys or Aib.

71. A GLP-1 receptor agonists comprising an amino acid sequence of Formula I, as defined above, wherein X₂₂ represents Gly, Ser, or Glu.

72. A GLP-1 receptor agonists comprising an amino acid sequence of Formula I, as defined above, wherein X₂₃ represents Gln, Glu, Lys, Trp, Arg, or Asp.

73. A GLP-1 receptor agonists comprising an amino acid sequence of Formula I, as defined above, wherein X₂₄ represents Ala, Aib, Lys, or Arg.

74. A GLP-1 receptor agonists comprising an amino acid sequence of Formula I, as defined above, wherein X₂₅ is Ala, Val, Phe, His, Leu, Met, Trp, Tyr, Ile or Aib.

75. A GLP-1 receptor agonists comprising an amino acid sequence of Formula I, as defined above, wherein X₂₅ represents Ala, Val, Leu, Ile, or Aib.

76. A GLP-1 receptor agonists comprising an amino acid sequence of Formula I, as defined above, wherein X₂₅ represents Ala, or Val.

77. A GLP-1 receptor agonists comprising an amino acid sequence of Formula I, as defined above, wherein X₂₆ represents Lys, Asn, Glu, Arg, His, Gly, Val, or Gln.

78. A GLP-1 receptor agonists comprising an amino acid sequence of Formula I, as defined above, wherein X₂₆ represents Lys, Arg, or Gln.

79. A GLP-1 receptor agonists comprising an amino acid sequence of Formula I, as defined above, wherein X₂₆ is Lys, Asn, Glu, Arg, His, Gly or Val.

80. A GLP-1 receptor agonists comprising an amino acid sequence of Formula I, as defined above, wherein X₂₆ represents Lys, Arg, or Gln.

81. A GLP-1 receptor agonists comprising an amino acid sequence of Formula I, as defined above, wherein X₂₆ represents Lys, or Arg.

82. A GLP-1 receptor agonists comprising an amino acid sequence of Formula I, as defined above, wherein X₂₇ is Glu, Asp, Gln, Ala, His, Gly, Arg, Lys, Aib or Leu.

83. A GLP-1 receptor agonists comprising an amino acid sequence of Formula I, as defined above, wherein X₂₇ represents Glu, Asp, Gln, Lys, or Leu.

84. A GLP-1 receptor agonists comprising an amino acid sequence of Formula I, as defined above, wherein X₂₉ represents Ile, or Val.

85. A GLP-1 receptor agonists comprising an amino acid sequence of Formula I, as defined above, wherein X₃₀ represents Ala, Val, Gln, Ile, Trp, Aib, Glu, Arg, or Lys.

86. A GLP-1 receptor agonists comprising an amino acid sequence of Formula I, as defined above, wherein X₃₀ represents Ala, Gln, Trp, Aib, Glu, or Lys.

87. A GLP-1 receptor agonists comprising an amino acid sequence of Formula I, as defined above, wherein X₃₀ is Ala, Val, Gln, Ile, Trp, Aib, Glu or Arg.

88. A GLP-1 receptor agonists comprising an amino acid sequence of Formula I, as defined above, wherein X₃₀ represents Ala, Gln, Aib, Glu, or Lys.

89. A GLP-1 receptor agonists comprising an amino acid sequence of Formula I, as defined above, wherein X₃₁ is Trp, Gln, Lys or His.

90. A GLP-1 receptor agonists comprising an amino acid sequence of Formula I, as defined above, wherein X₃₁ represents Trp, or His.

91. A GLP-1 receptor agonists comprising an amino acid sequence of Formula I, as defined above, wherein X₃₃ is Val, Met, Ile, Leu, Thr, Arg or Lys.

92. A GLP-1 receptor agonists comprising an amino acid sequence of Formula I, as defined above, wherein X₃₃ represents Val, Ile, Leu, Thr, Arg, or Lys.

93. A GLP-1 receptor agonists comprising an amino acid sequence of Formula I, as defined above, wherein X₃₃ represents Val, Leu, or Lys.

94. A GLP-1 receptor agonists comprising an amino acid sequence of Formula I, as defined above, wherein X₃₃ represents Val, Met, Leu, or Lys.

95. A GLP-1 receptor agonists comprising an amino acid sequence of Formula I, as defined above, wherein X₃₄-X₃₅-X₃₆-X₃₇-X₃₈-X₃₉ represents Subsequence 1, composed by the following amino acid residues “Glu-Lys-Aib-Lys-Glu-Phe”, and X₇-X₃₃, and X₄₀-X₅₀ are as defined herein.

96. A GLP-1 receptor agonists comprising an amino acid sequence of Formula I, as defined above, wherein X₃₄-X₃₅-X₃₆-X₃₇-X₃₈-X₃₉-X₄₀ represents Subsequence 2, composed of the following amino acid residues “Glu-Lys-Aib-Lys-Glu-Phe-Leu”, and and X₇-X₃₃, and X₄₁-X₅₀ are as defined herein.

97. A GLP-1 receptor agonists comprising an amino acid sequence of Formula I, as defined above, wherein X₃₄ is Lys, Glu, Asn, Asp, Gln, His, Gly or Arg.

98. A GLP-1 receptor agonists comprising an amino acid sequence of Formula I, as defined above, wherein X₃₄ represents Lys, Glu, or Asn.

99. A GLP-1 receptor agonists comprising an amino acid sequence of Formula I, as defined above, wherein X₃₄ represents Asn, Gln, Lys, His, Gly, Arg, or Asp.

100. A GLP-1 receptor agonists comprising an amino acid sequence of Formula I, as defined above, wherein X₃₄ represents Asn, Gln, Lys, or His.

101. A GLP-1 receptor agonists comprising an amino acid sequence of Formula I, as defined above, wherein X₃₅ is Gly, Lys, Arg, His, Ser, Thr or Aib.

102. A GLP-1 receptor agonists comprising an amino acid sequence of Formula I, as defined above, wherein X₃₅ represents Gly, Lys, Arg, or Thr.

103. A GLP-1 receptor agonists comprising an amino acid sequence of Formula I, as defined above, wherein X₃₅ represents [Arg, Ala, His, Gln, Gly, Asn, or Aib.

104. A GLP-1 receptor agonists comprising an amino acid sequence of Formula I, as defined above, wherein X₃₅ represents [Arg, Ala, His, or Gln

105. A GLP-1 receptor agonists comprising an amino acid sequence of Formula I, as defined above, wherein X₃₆ is Gly, Aib, Val, Leu, Ala, His, Ile, Met, Trp, Tyr or Phe.

106. A GLP-1 receptor agonists comprising an amino acid sequence of Formula I, as defined above, wherein X₃₆ is a lipophilic residue or Gly.

107. A GLP-1 receptor agonists comprising an amino acid sequence of Formula I, as defined above, wherein X₃₆ represents Gly, Aib, Val, Leu, or Phe.

108. A GLP-1 receptor agonists comprising an amino acid sequence of Formula I, as defined above, wherein X₃₆ represents Gly, Val, Leu, Phe, Ile, Trp, Tyr, Ala, Met, or His.

109. A GLP-1 receptor agonists comprising an amino acid sequence of Formula I, as defined above, wherein X₃₆ represents Gly, Val, Leu, or Phe.

110. The GLP-1 receptor agonist peptide according to any one of the previous embodiments, wherein X₃₇ is Gly, Ala, Glu, Aib, His, Arg, Leu, Pro or Lys.

111. A GLP-1 receptor agonists comprising an amino acid sequence of Formula I, as defined above, wherein X₃₇ represents Gly, Ala, Arg, Leu, Pro, or Lys.

112. A GLP-1 receptor agonists comprising an amino acid sequence of Formula I, as defined above, wherein X₃₇ represents Arg, Ala, Leu, Gly, His, Gln, Asn, Aib, Ile, Val, or Phe.

113. A GLP-1 receptor agonists comprising an amino acid sequence of Formula I, as defined above, wherein X₃₇ represents Arg, Ala, Leu, Gly, His, or Gln.

114. The GLP-1 receptor agonist peptide according to any one of the previous embodiments, wherein X₃₈ is Glu, Ser, Asp, His, or amide, or X₃₈ is absent.

115. A GLP-1 receptor agonists comprising an amino acid sequence of Formula I, as defined above, wherein X₃₈ is Glu, Ser, Asp or His.

116. A GLP-1 receptor agonists comprising an amino acid sequence of Formula I, as defined above, wherein X₃₈ represents Glu, Ser, or amide, or X₃₈ is absent.

117. A GLP-1 receptor agonists comprising an amino acid sequence of Formula I, as defined above, wherein X₃₈ represents Asp, His, Gln, Ser, Gly, Asn, or Thr.

118. A GLP-1 receptor agonists comprising an amino acid sequence of Formula I, as defined above, wherein X₃₈ represents Asp, His, Gln, Ser, or Gly.

119. A GLP-1 receptor agonists comprising an amino acid sequence of Formula I, as defined above, wherein X₃₉ is Phe, Leu, His, Ala, Ser, Ile, Met, Val, Trp, Tyr, or amide, or X₃₉ is absent.

120. A GLP-1 receptor agonists comprising an amino acid sequence of Formula I, as defined above, wherein X₃₉ is Phe, Leu, His, Ala, Ser, Ile, Met, Val, Trp or Tyr.

121. A GLP-1 receptor agonists comprising an amino acid sequence of Formula I, as defined above, wherein X₃₉ represents Phe, or Ser, or X₃₉ is absent.

122. A GLP-1 receptor agonists comprising an amino acid sequence of Formula I, as defined above, wherein X₃₉ represents Trp, Ala, Glu, Leu, Val, Gly, His, Lys, Ser, Thr, Tyr, Aib, Ile, or Met.

123. A GLP-1 receptor agonists comprising an amino acid sequence of Formula I, as defined above, wherein X₃₉ represents Trp, Ala, Glu, Leu, Val, Gly, His, Lys, or Ser.

124. A GLP-1 receptor agonists comprising an amino acid sequence of Formula I, as defined above, wherein X₄₀ is Leu, Phe, Val, His, Gly, Ala, Ile, Met, Trp, Tyr, or amide, or X₄₀ is absent.

125. A GLP-1 receptor agonists comprising an amino acid sequence of Formula I, as defined above, wherein X₄₀ is Leu, Phe, Val, His, Gly, Ala, Ile, Met, Trp or Tyr.

126. A GLP-1 receptor agonists comprising an amino acid sequence of Formula I, as defined above, wherein X₄₀ represents Leu, Gly, or amide, or X₄₀ is absent.

127. A GLP-1 receptor agonists comprising an amino acid sequence of Formula I, as defined above, wherein X₄₀ represents Leu, Phe, Val, His, Tyr, or amide, or X₄₀ is absent.

128. A GLP-1 receptor agonists comprising an amino acid sequence of Formula I, as defined above, wherein X₄₁ is Glu, Ala, or amide, or X₄₁ is absent.

129. A GLP-1 receptor agonists comprising an amino acid sequence of Formula I, as defined above, wherein X₄₁ represents Glu, or Ala, or X₄₁ is absent.

130. A GLP-1 receptor agonists comprising an amino acid sequence of Formula I, as defined above, wherein X₄₁ represents Glu, Asp, Ala, Gly, Lys, or amide, or X₄₁ is absent.

131. A GLP-1 receptor agonists comprising an amino acid sequence of Formula I, as defined above, wherein X₄₂ is Leu, Pro, Lys, or amide, or X₄₂ is absent.

132. A GLP-1 receptor agonists comprising an amino acid sequence of Formula I, as defined above, wherein X₄₂ represents Leu, or Pro, or X₄₂ is absent.

133. A GLP-1 receptor agonists comprising an amino acid sequence of Formula I, as defined above, wherein X₄₂ represents Leu, Pro, Lys, Arg, or amide, or X₄₂ is absent.

134. A GLP-1 receptor agonists comprising an amino acid sequence of Formula I, as defined above, wherein X₄₃ is Leu, Pro, Val, or amide, or X₄₃ is absent.

135. A GLP-1 receptor agonists comprising an amino acid sequence of Formula I, as defined above, wherein X₄₃ represents Leu, or Pro, or X₄₃ is absent.

136. A GLP-1 receptor agonists comprising an amino acid sequence of Formula I, as defined above, wherein X₄₃ represents Leu, Pro, Val, or amide, or X₄₃ is absent.

137. A GLP-1 receptor agonists comprising an amino acid sequence of Formula I, as defined above, wherein X₄₄ is Lys, or amide, or X₄₄ is absent.

138. A GLP-1 receptor agonists comprising an amino acid sequence of Formula I, as defined above, wherein X₄₅ is Glu, or amide, or X₄₅ is absent.

139. A GLP-1 receptor agonists comprising an amino acid sequence of Formula I, as defined above, wherein X₄₅ is Glu, or X₄₅ is absent.

140. A GLP-1 receptor agonists comprising an amino acid sequence of Formula I, as defined above, wherein X₄₆ is Phe, Ile, or amide, or X₄₆ is absent.

141. A GLP-1 receptor agonists comprising an amino acid sequence of Formula I, as defined above, wherein X₄₆ is absent.

142. A GLP-1 receptor agonists comprising an amino acid sequence of Formula I, as defined above, wherein X₄₇ is Ile, or amide, or X₄₇ is absent.

143. A GLP-1 receptor agonists comprising an amino acid sequence of Formula I, as defined above, wherein X₄₇ is absent.

144. A GLP-1 receptor agonists comprising an amino acid sequence of Formula I, as defined above, wherein X₄₈ is Ala, or amide, or X₄₈ is absent.

145. A GLP-1 receptor agonists comprising an amino acid sequence of Formula I, as defined above, wherein X₄₈ is absent.

146. A GLP-1 receptor agonists comprising an amino acid sequence of Formula I, as defined above, wherein X₄₉ is Trp, or amide, or X₄₉ is absent.

147. A GLP-1 receptor agonists comprising an amino acid sequence of Formula I, as defined above, wherein X₄₉ is absent.

148. A GLP-1 receptor agonists comprising an amino acid sequence of Formula I, as defined above, wherein X₅₀ is amide, or X₅₀ is absent.

149. A GLP-1 receptor agonists comprising an amino acid sequence of Formula I, as defined above, wherein X₅₀ is absent.

150. A GLP-1 receptor agonists comprising an amino acid sequence of Formula I, as defined above, wherein all of positions X₄₆-X₅₀, all of positions X₄₅-X₅₀, all of positions X₄₄-X₅₀, or all of positions X₄₃-X₅₀, are absent.

151. A GLP-1 receptor agonists comprising an amino acid sequence of Formula I, as defined above, wherein at least 4, at least 5, or at least 6 of the following substitutions are present: X₂₃ is a charged amino acid residue, X₂₅ is a lipophilic amino acid residue, X₂₇ is a negatively charged amino acid residue, X₃₄ is a negatively charged amino acid residue, X₃₅ is a positively charged amino acid residue, X₃₇ is a positively charged amino acid residue, X₃₈ is a negatively charged amino acid residue, X₃₉ is a lipophilic amino acid residue, and/or X₄₀ is a lipophilic amino acid residue.

152. A GLP-1 receptor agonists comprising an amino acid sequence of Formula I, as defined above, wherein at least 2, at least 3 or at least 4, of the following substitutions are present: X₂₃ is a charged amino acid residue, X₂₅ is a lipophilic amino acid residue, X₂₇ is an negatively charged amino acid residue, X₃₄ is an negatively charged amino acid residue, X₃₅ is a positively charged amino acid residue, X₃₆ is a lipophilic amino acid residue, X₃₈ is an negatively charged amino acid residue, X₃₉ is a lipophilic amino acid residue, and/or X₄₀ is a lipophilic amino acid residue.

153. A GLP-1 receptor agonists comprising an amino acid sequence of Formula I, as defined above, wherein at least 4 or at least 5 of X₂₅, X₂₉, X₃₆, X₃₉ and X₄₀ are lipophilic amino acid residues.

154. A GLP-1 receptor agonists comprising an amino acid sequence of Formula I, as defined above, wherein at least 4, at least 5, at least 6 or at least 7 of X₂₃, X₂₆, X₂₇, X₃₀, X₃₃, X₃₄, X₃₅, X₃₇ and X₃₈ are polar amino acid residues.

155. A GLP-1 receptor agonists comprising an amino acid sequence of Formula I, as defined above, wherein at least 4 or at least 5 of X₂₃, X₂₄, X₂₆, X₂₇, X₃₀, X₃₁, X₃₃, X₃₄, X₃₅, X₃₇ and X₃₈ are charged amino acid residues.

156. A GLP-1 receptor agonists comprising an amino acid sequence of Formula I, as defined above, wherein at least 2 of X₂₄, X₂₆, X₃₃, X₃₅ and X₃₇ are positively charged residues.

157. A GLP-1 receptor agonists comprising an amino acid sequence of Formula I, as defined above, wherein at least 2 of X₂₃, X₂₇, X₃₄ and X₃₈ are negatively charged residues.

158. A GLP-1 receptor agonists comprising an amino acid sequence of Formula I, as defined above, wherein X₇-X₄₀ represent positions 1 through 33 of SEQ ID 9, with up to 10 conservative mutations.

159. A GLP-1 receptor agonists comprising an amino acid sequence of Formula I, as defined above, wherein X₇-X₄₀ represent positions 1 through 33 of SEQ ID 9, with up to 9 conservative mutations.

160. A GLP-1 receptor agonists comprising an amino acid sequence of Formula I, as defined above, wherein X₇-X₄₀ represent positions 1 through 33 of SEQ ID 9, with up to 8 conservative mutations.

161. A GLP-1 receptor agonists comprising an amino acid sequence of Formula I, as defined above, wherein X₇-X₄₀ represent positions 1 through 33 of SEQ ID 9, with up to 7 conservative mutations.

162. A GLP-1 receptor agonists comprising an amino acid sequence of Formula I, as defined above, wherein X₇-X₄₀ represent positions 1 through 33 of SEQ ID 9, with up to 6 conservative mutations.

163. A GLP-1 receptor agonists comprising an amino acid sequence of Formula I, as defined above, wherein X₇-X₄₀ represent positions 1 through 33 of SEQ ID 9, with up to 5 conservative mutations.

164. A GLP-1 receptor agonists comprising an amino acid sequence of Formula I, as defined above, wherein X₇-X₄₀ represent positions 1 through 33 of SEQ ID 10, with up to 10 conservative mutations.

165. A GLP-1 receptor agonists comprising an amino acid sequence of Formula I, as defined above, wherein X₇-X₄₀ represent positions 1 through 33 of SEQ ID 10, with up to 9 conservative mutations.

166. A GLP-1 receptor agonists comprising an amino acid sequence of Formula I, as defined above, wherein X₇-X₄₀ represent positions 1 through 33 of SEQ ID 10, with up to 8 conservative mutations.

167. A GLP-1 receptor agonists comprising an amino acid sequence of Formula I, as defined above, wherein X₇-X₄₀ represent positions 1 through 33 of SEQ ID 10, with up to 7 conservative mutations.

168. A GLP-1 receptor agonists comprising an amino acid sequence of Formula I, as defined above, wherein X₇-X₄₀ represent positions 1 through 33 of SEQ ID 10, with up to 6 conservative mutations.

169. A GLP-1 receptor agonists comprising an amino acid sequence of Formula I, as defined above, wherein X₇-X₄₀ represent positions 1 through 33 of SEQ ID 10, with up to 5 conservative mutations.

170. A GLP-1 receptor agonists comprising an amino acid sequence of Formula I, as defined above, wherein X₇-X₄₀ represent positions 1 through 33 of SEQ ID 11, with up to 10 conservative mutations.

171. A GLP-1 receptor agonists comprising an amino acid sequence of Formula I, as defined above, wherein X₇-X₄₀ represent positions 1 through 33 of SEQ ID 11, with up to 9 conservative mutations.

172. A GLP-1 receptor agonists comprising an amino acid sequence of Formula I, as defined above, wherein X₇-X₄₀ represent positions 1 through 33 of SEQ ID 11, with up to 8 conservative mutations.

173. A GLP-1 receptor agonists comprising an amino acid sequence of Formula I, as defined above, wherein X₇-X₄₀ represent positions 1 through 33 of SEQ ID 11, with up to 7 conservative mutations.

174. A GLP-1 receptor agonists comprising an amino acid sequence of Formula I, as defined above, wherein X₇-X₄₀ represent positions 1 through 33 of SEQ ID 11, with up to 6 conservative mutations.

175. A GLP-1 receptor agonists comprising an amino acid sequence of Formula I, as defined above, wherein X₇-X₄₀ represent positions 1 through 33 of SEQ ID 11, with up to 5 conservative mutations.

176. The GLP-1 receptor agonist peptide according to any one of embodiments 158-175 holding up to 45 amino acid residues, or up to 44 amino acid residues, or up to 43 amino acid residues, or up to 42 amino acid residues, or up to 41 amino acid residues, or up to 40 amino acid residues.

177. The GLP-1 receptor agonist peptide according to any one of the previous embodiments, with a cholesterol efflux in vitro EC₅₀ potency of less than 3 μM, less than 2 μM, less than 1 μM or less than 0.5 μM.

178. The GLP-1 receptor agonist peptide according to embodiment 177, with a cholesterol efflux in vitro EC₅₀ potency of less than 2 μM.

179. The GLP-1 receptor agonist peptide according to embodiment 177, with a cholesterol efflux in vitro EC₅₀ potency of less than 1 μM.

180. The GLP-1 receptor agonist peptide according to embodiment 177, with a cholesterol efflux in vitro EC₅₀ potency of less than 0.5 μM.

181. The GLP-1 receptor agonist peptide according to any one of the previous embodiments, with a GLP-1 in vitro potency of at least 25%, between 10% and 25%, or between 1% and 10% of that of native GLP-1.

182. The GLP-1 receptor agonist peptide according to embodiment 181, with a GLP-1 in vitro potency of at least 25% of that of native GLP-1.

183. The GLP-1 receptor agonist peptide according to embodiment 181, with a GLP-1 in vitro potency of between 10% and 25% of that of native GLP-1.

184. The GLP-1 receptor agonist peptide according to embodiment 181, with a GLP-1 in vitro potency of between 1% and 10% of that of native GLP-1.

185. The GLP-1 receptor agonist peptide according to any one of the previous embodiments, with a cholesterol efflux in vitro EC₅₀ potency of less than 3 μM and a GLP-1 in vitro potency of at least 25%, between 10% and 25% or between 1% and 10% of that of GLP-1.

186. The GLP-1 receptor agonist peptide according to embodiment 185, with a cholesterol efflux in vitro EC₅₀ potency of less than 2 μM and a GLP-1 in vitro potency of at least 25%, between 10% and 25% or between 1% and 10% of that of GLP-1.

187. The GLP-1 receptor agonist peptide according to embodiment 185, with a cholesterol efflux in vitro EC₅₀ potency of less than 1 μM and a GLP-1 in vitro potency of at least 25%, between 10% and 25% or between 1% and 10% of that of GLP-1.

188. The GLP-1 receptor agonist peptide according to embodiment 185, with a cholesterol efflux in vitro EC₅₀ potency of less than 0.5 μM and a GLP-1 in vitro potency of at least 25%, between 10% and 25% or between 1% and 10% of that of GLP-1.

189. The GLP-1 receptor agonist peptide according to any one of the previous embodiments, with a ratio between cholesterol efflux Eₘₐₓ and EC₅₀ (Eₘₐₓ/EC₅₀) of at least 30%/μM, at least 50%/μM, or at least 100%/μM.

190. The GLP-1 receptor agonist peptide according to embodiment 189, with a ratio between cholesterol efflux Eₘₐₓ and EC₅₀ (Eₘₐₓ/EC₅₀) of at least 30%/μM.

191. The GLP-1 receptor agonist peptide according to embodiment 189, with a ratio between cholesterol efflux Eₘₐₓ and EC₅₀ (Eₘₐₓ/EC₅₀) of at least 50%/μM.

192. The GLP-1 receptor agonist peptide according to embodiment 189, with a ratio between cholesterol efflux Eₘₐₓ and EC₅₀ (Eₘₐₓ/EC₅₀) of at least 100%/μM.

193. The GLP-1 receptor agonist peptide according to any one of the previous embodiments, which shows an in vitro Eₘₐₓ, as determined by the method of Example 6, at or above 65% of the Eₘₐₓ of L-4F; or at or above 75% of the Eₘₐₓ of L-4F.

194. The GLP-1 receptor agonist peptide according to any one of the previous embodiments, wherein said GLP-1 receptor agonist peptide has been fused to a peptide comprising at least one, and up to four ApoA-I mimetic peptide sequence(s), and which optionally ends as a C-terminal amide.

195. The GLP-1 receptor agonist peptide according to embodiment 194, wherein said GLP-1 receptor agonist peptide has been fused to a peptide comprising one ApoA-I mimetic peptide sequence and which optionally ends as a C-terminal amide.

196. The GLP-1 receptor agonist peptide according to embodiment 194, wherein said GLP-1 receptor agonist peptide has been fused to a peptide comprising two ApoA-I mimetic peptide sequences and which optionally ends as a C-terminal amide.

197. The GLP-1 receptor agonist peptide according to embodiment 194, wherein said GLP-1 receptor agonist peptide has been fused to a peptide comprising three ApoA-I mimetic peptide sequences and which optionally ends as a C-terminal amide.

198. The GLP-1 receptor agonist peptide according to embodiment 194, wherein said GLP-1 receptor agonist peptide has been fused to a peptide comprising four ApoA-I mimetic peptide sequences and which optionally ends as a C-terminal amide.

199. The GLP-1 receptor agonist peptide according to any one of embodiments 194-198, wherein at least one of said ApoA-I mimetic peptide is selected from SEQ ID 6, SEQ ID 7 SEQ ID 8, SEQ ID 14 or SEQ ID 15 with up to 18 conservative substitutions.

200. The GLP-1 receptor agonist peptide according to any one of embodiments 194-198, wherein at least one of said ApoA-I mimetic peptide is selected from SEQ ID 6, SEQ ID 7 SEQ ID 8, SEQ ID 14 or SEQ ID 15 with up to 14 conservative substitutions.

201. The GLP-1 receptor agonist peptide according to any one of embodiments 194-198, wherein at least one of said ApoA-I mimetic peptide is selected from SEQ ID 6, SEQ ID 7 SEQ ID 8, SEQ ID 14 or SEQ ID 15 with up to 10 conservative substitutions.

202. The GLP-1 receptor agonist peptide according to any one of embodiments 194-198, wherein at least one of said ApoA-I mimetic peptide is selected from SEQ ID 6, SEQ ID 7 SEQ ID 8, SEQ ID 14 or SEQ ID 15 with up to 5 conservative substitutions.

203. The GLP-1 receptor agonist peptide according to any one of embodiments 194-198, wherein at least one of said ApoA-I mimetic peptide is selected from SEQ ID 6, SEQ ID 7 SEQ ID 8, SEQ ID 14 or SEQ ID 15 with up to 2 conservative substitutions.

204. The GLP-1 receptor agonist peptide according to any one of embodiments 194-198, wherein at least one of said ApoA-I mimetic peptide is selected from SEQ ID 6, SEQ ID 7 SEQ ID 8, SEQ ID 14, or SEQ ID 15, with up to 1 conservative substitution.

205. The GLP-1 receptor agonist peptide according to embodiment 194, wherein at least one of said ApoA-I mimetic peptide sequence(s) is SEQ ID 6, SEQ ID 7, SEQ ID 8, SEQ ID 14, or SEQ ID 15.

206. The GLP-1 receptor agonist peptide according to embodiment 205, wherein at least one of said ApoA-I mimetic peptide is SEQ ID 6.

207. The GLP-1 receptor agonist peptide according to embodiment 205, wherein at least one of said ApoA-I mimetic peptide is SEQ ID 7.

208. The GLP-1 receptor agonist peptide according to embodiment 205, wherein at least one of said ApoA-I mimetic peptide is SEQ ID 8.

209. The GLP-1 receptor agonist peptide according to embodiment 205, wherein at least one of said ApoA-I mimetic peptide is SEQ ID 14.

210. The GLP-1 receptor agonist peptide according to embodiment 205, wherein at least one of said ApoA-I mimetic peptide is SEQ ID 15.

211. A GLP-1 receptor agonist selected from the group consisting of:

[Aib8,Glu23,Val25,Glu34,Lys35,Aib36,Lys37]-GLP-1-(7-37)-peptidyl-Glu-Phe-Leu amide;

[Aib2,Gly16,Glu21,Lys29,Aib30,Leu31,Glu32,Phe33,Leu34,Glu35,Leu36,Leu37]-Exendin-4-(1-37)-peptide amide;

[Aib8,Glu23,Aib24,Val25,Aib30,Glu34,Lys35,Aib36,Lys37]-GLP-1-(7-37)-peptidyl-Glu-Phe-Leu amide;

[Aib8,Glu23,Val25,Glu34,Lys35,Aib36,Lys37]-GLP-1-(7-37)-peptidyl-Glu-His-Leu amide;

[Aib8,Glu23,Val25,Glu34,Lys35,Val36,Lys37]-GLP-1-(7-37)-peptidyl-Glu-His-Leu amide;

[Aib8,Glu23,Val25,Glu34,Lys35,Val36,Lys37]-GLP-1-(7-37)-peptidyl-Glu-Phe-Leu amide;

[Aib8,Val25,Glu34,Lys35,Aib36,Lys37]-GLP-1-(7-37)-peptidyl-Glu-Phe-Leu amide;

[Aib8,Glu23,Glu34,Lys35,Aib36,Lys37]-GLP-1-(7-37)-peptidyl-Glu-Phe-Leu amide;

[Aib8,Glu23,Val25,Lys35,Aib36,Lys37]-GLP-1-(7-37)-peptidyl-Glu-Phe-Leu amide;

[Aib8,Glu23,Val25,Aib36,Lys37]-des-Lys34-GLP-1-(7-37)-peptidyl-Glu-Phe-Leu amide;

[Aib8,Glu23,Val25,Glu34,Lys35,Aib36,Lys37]-GLP-1-(7-37)-peptide amide;

[Aib2,Gly16,Lys29,Aib30,Leu31,Glu32,Phe33,Leu34,Glu35,Leu36,Leu37]-Exendin-4-(1-37)-peptide amide;

[Tyr12,Asn14,Thr17,Glu18,Glu23,Val25,Glu34,Lys35,Aib36,Lys37]-GLP-1-(7-37)-peptidyl-Glu-Phe-Leu amide;

[Asp9,Leu12,Ile16,Glu23,Val25,Glu34,Lys35,Aib36,Lys37]-GLP-1-(7-37)-peptidyl-Glu-Phe-Leu amide;

[His14,Tyr20,Glu23,Val25,Glu34,Lys35,Aib36,Lys37]-GLP-1-(7-37)-peptidyl-Glu-Phe-Leu amide;

[Aib8,Glu23,Val25,Glu34,Lys35,Aib36,Lys37]-GLP-1-(7-37)-peptidyl-Glu-Phe-Leu;

[Aib8,Glu23,Val25,His31,Glu34,Lys35,Aib36,Lys37]-GLP-1-(7-37)-peptidyl-Glu-Phe-Leu amide;

[Aib8,Asp23,Val25,Asp27,Glu34,Lys35,Aib36,Lys37]-GLP-1-(7-37)-peptidyl-Glu-Phe-Leu amide;

[Aib8,Glu23,Val25,Arg26,Glu34,Arg35,Aib36,Lys37]-GLP-1-(7-37)-peptidyl-Glu-Phe-Leu amide;

[Aib8,Glu23,Val25,Glu34,Lys35,Leu36,Lys37]-GLP-1-(7-37)-peptidyl-Glu-Leu-Val amide;

[Aib8,Trp23,Val25,Glu34,Lys35,Aib36,Lys37]-GLP-1-(7-37)-peptidyl-Glu-Phe-Leu amide;

[Aib8,Glu23,Val25,Glu34,Lys35,Aib36,Lys37]-GLP-1-(7-37)-peptidyl-Glu-Phe-His amide;

[Aib2,Glu21,Lys29,Aib30,Leu31,Glu32,Phe33,Leu34,Glu35,Leu36,Leu37]-Exendin-4-(1-37)-peptide;

[Aib2,Gly16,Lys17,Ala18,Arg20,Glu21,Leu27,Glu28,Lys29]-Glucagonyl-(1-29)-Aib-Lys-Glu-Phe-Leu amide;

[Aib2,Gly16,Lys17,Arg20,Glu21,Leu27,Glu28,Lys29]-Glucagonyl-(1-29)-Aib-Lys-Glu-Phe-Leu amide;

[Aib2,Lys17,Ala18,Arg20,Glu21,Leu27,Glu28,Lys29]-Glucagonyl-(1-29)-Aib-Lys-Glu-Phe-Leu amide;

[Asn14,Met16,Thr17,Asn18,Glu23,Val25,Glu34,Lys35,Gly36,Lys37]-GLP-1-(7-37)-peptidyl-Glu-Phe-Leu amide;

[Aib8,Glu23,Val25,Leu27,Glu34,Lys35,Aib36,Lys37]-GLP-1-(7-37)-peptidyl-Glu-Phe-Leu amide;

[Aib2,Glu21,Lys29,Aib30,Leu31,Glu32,Phe33,Leu34,Glu35,Leu36,Leu37]-Exendin-4-(1-37)-peptide amide;

[Aib8,Glu23,Val25,Glu34,Aib36,Lys37]-GLP-1-(7-37)-peptidyl-Glu-Phe-Leu amide;

[Aib8,Glu23,Val25,Glu34,Lys35,Aib36,Lys37]-GLP-1-(7-37)-peptidyl-His-Phe-Leu amide;

[Aib8,Glu23,Val25,Glu34,Lys35,Aib36]-GLP-1-(7-37)-peptidyl-Glu-Phe-Leu amide;

[Aib2,Glu21,Lys29,Leu31,Glu32,Phe33,Leu34,Glu35,Leu36,Leu37]-Exendin-4-(1-37)-peptide amide;

[Aib2,Glu21,Aib30,Leu31,Glu32,Phe33,Leu34,Glu35,Leu36,Leu37]-Exendin-4-(1-37)-peptide amide;

[Aib2,Glu21,Lys29,Aib30,Glu32,Phe33,Leu34,Glu35,Leu36,Leu37]-Exendin-4-(1-37)-peptide amide;

[Aib2,Glu21,Lys29,Aib30,Leu31,Phe33,Leu34,Glu35,Leu36,Leu37]-Exendin-4-(1-37)-peptide amide;

[Aib2,Glu21,Lys29,Aib30,Leu31,Glu32,Leu34,Glu35,Leu36,Leu37]-Exendin-4-(1-37)-peptide amide;

[Aib2,Glu21,Lys29,Aib30,Leu31,Glu32,Phe33,Glu35,Leu36,Leu37]-Exendin-4-(1-37)-peptide amide;

[Aib2,Glu21,Lys29,Aib30,Leu31,Glu32,Phe33,Leu34,Glu35,Leu37]-Exendin-4-(1-37)-peptide amide;

[Aib2,Glu21,Lys29,Aib30,Leu31,Glu32,Phe33,Leu34,Glu35,Leu36]-Exendin-4-(1-37)-peptide amide;

[Aib2,Glu21,Lys29,Aib30,Leu31,Glu32,Phe33,Leu34,Leu36,Leu37]-Exendin-4-(1-37)-peptide amide;

[Aib2,Glu21,Lys29,Aib30,Leu31,Glu32,Phe33,Leu34]-Exendin-4-(1-34)-peptide amide;

[Aib8,Glu23,Val25,Arg26,Glu34,Arg35,Aib36,Arg37]-GLP-1-(7-37)-peptidyl-Glu-Phe-Leu amide;

[Asn14,Met16,Thr17,Asn18,Glu23,Val25,Glu34,Lys35,Aib36,Lys37]-GLP-1-(7-37)-peptidyl-Glu-Phe-Leu amide;

[Aib8,Glu34,Lys35,Aib36,Lys37]-GLP-1-(7-37)-peptidyl-Glu-Phe-Leu amide;

[Aib8,Glu23,Val25,Glu34,Lys35,Phe36,Lys37]-GLP-1-(7-37)-peptidyl-Glu-Ala-Phe amide;

[Aib8,Lys35,Aib36,Lys37]-GLP-1-(7-37)-peptidyl-Glu-Phe-Leu amide;

[Aib8,Lys35,Aib36]-GLP-1-(7-37)-peptidyl-Glu-Phe-Leu amide;

[Aib8,Glu23,Lys24,Val25,Glu30,Glu34,Lys35,Aib36,Lys37]-GLP-1-(7-37)-peptidyl-Glu-Phe-Leu amide;

[Aib8,Glu23,Val25,Trp30,Glu34,Lys35,Aib36,Lys37]-GLP-1-(7-37)-peptidyl-Glu-Phe-Leu amide;

[Aib8,Glu23,Val25,Lys27,Glu34,Lys35,Aib36,Lys37]-GLP-1-(7-37)-peptidyl-Glu-Phe-Leu amide;

[Aib8,Lys23,Arg24,Arg26,Glu34,Lys35,Aib36,Lys37]-GLP-1-(7-37)-peptidyl-Glu-Phe-Leu amide;

[Aib8,Glu23,Arg24,Val25,Glu34,Lys35,Aib36,Lys37]-GLP-1-(7-37)-peptidyl-Glu-Phe-Leu amide;

[Aib8,Glu23,Val25,Glu34,Lys35,Aib36,Lys37]-GLP-1-(7-37)-peptidyl-Glu-Phe-Leu-Glu-Lys-Val amide;

[Aib2,His3,Glu15,Glu16,Glu17,Ala18,Lys20,Glu21,Ile23,Ala24,Leu27,Glu28,Lys29]-Glucagonyl-(1-29)-Aib-Lys-GLu-Phe-Leu amide;

[Glu15,Glu16,Gln17,Ala18,Lys20,Glu21,Ile23,Ala24,Leu27,Glu28,Lys29]-Glucagonyl-(1-29)-Aib-Lys-Glu-Phe-Leu amide;

[Glu15,Glu16,Lys17,Ala18,Lys20,Glu21,Ile23,Ala24,Leu27,Glu28,Lys29]-Glucagonyl-(1-29)-Aib-Lys-Glu-Phe-Leu amide;

[Glu15,Glu16,Lys17,Lys18,Lys20,Glu21,Ile23,Ala24,Leu27,Glu28,Lys29]-Glucagonyl-(1-29)-Aib-Lys-Glu-Phe-Leu amide;

[Lys17,Ala18,Arg20,Glu21,Leu27,Glu28,Lys29]-Glucagonyl-(1-29)-Aib-Lys-Glu-Phe-Leu amide;

[Aib8,Glu23,Lys24,Val25,Glu34,Lys35,Aib36,Lys37]-GLP-1-(7-37)-peptidyl-Glu-Phe-Leu amide;

[Aib8,Glu23,Val25,Glu34,Lys35,Aib36,Lys37]-GLP-1-(7-37)-peptidyl-Glu-Phe-Leu-Glu-Lys-Val-Lys-Glu-Phe amide;

[Aib8,Glu23,Val25,Glu34,Lys35,Aib36,Lys37]-GLP-1-(7-37)-peptidyl-Glu-Phe amide;

[Aib8,Glu23,Val25,Glu34,Lys35,Aib36,Lys37]-GLP-1-(7-37)-peptidyl-Glu amide;

[Aib8,Glu23,Lys24,Val25,Glu34,Aib36,Lys37]-GLP-1-(7-37)-peptidyl-Glu-Phe-Leu amide;

[Aib8,Glu23,Val25,Val29,Gln30,Glu34,Lys35,Aib36,Lys37]-GLP-1-(7-37)-peptidyl-Glu-Phe-Leu amide;

[Aib8,Glu23,Val25,Glu34,Lys35,Aib36,Lys37]-GLP-1-(7-37)-peptidyl-Glu-Phe-Leu-Glu amide;

[Glu23,Val25,Glu34,Lys35,Val36,Lys37]-GLP-1-(7-37)-peptidyl-Glu-Phe-Leu amide;

[Ala2,Lys17,Ala18,Arg20,Glu21,Leu27,Glu28,Lys29]-Glucagonyl-(1-29)-Val-Lys-Glu-Phe-Leu amide;

[Ala2,Glu21,Lys29,Va130,Leu31,Glu32,Phe33,Leu34]-Exendin-4-(1-34)-peptide amide;

[Aib8,Glu23,Val25, GLu34,Lys35,Aib36,Lys37]-GLP-1-(7-37)-peptidyl-Glu-Phe-Leu-Glu-Lys amide;

[Aib8,Glu23,Lys24,Val25, GLu30,His31,Glu34,Lys35,Aib36,Lys37]-GLP-1-(7-37)-peptidyl-Glu-Phe-Leu amide;

[Aib8,Val25,Gln27,Glu34,Lys35,Aib36,Ala37]-GLP-1-(7-37)-peptidyl-Glu-Phe-Leu amide;

[Aib8,Val25,Asn34,Lys35,Aib36,Ala37]-GLP-1-(7-37)-peptidyl-Glu-Phe-Leu amide;

[Aib8,Val25,Gln27,Asn34,Lys35,Aib36,Ala37]-GLP-1-(7-37)-peptidyl-Glu-Phe-Leu amide;

[Aib8,Val36]-GLP-1-(7-37)-peptidyl-Glu-Phe-Leu amide;

[Gly8,Glu23,Val25,Glu34,Lys35,Val36,Lys37]-GLP-1-(7-37)-peptidyl-Glu-Phe-Leu amide;

[desamino-His7,Aib8,Glu23,Val25,Glu34,Lys35,Val36,Lys37]-GLP-1-(7-37)-peptidyl-Glu-Phe-Leu amide;

[Gly8,Glu23,Val25,Glu34,Lys35,Val36,Lys37]-GLP-1-(7-37)-peptidyl-Glu-His-Leu amide;

[Aib8,Glu23,Val25, GI n30,Leu33,Glu34,Lys35,Aib36,Lys37]-GLP-1-(7-37)-peptidyl-Glu-Phe-Leu amide;

[Aib8,Glu23,Val25,Arg26,Val29, GIn30,Leu33,Glu34,Lys35,Aib36,Lys37]-GLP-1-(7-37)-peptidyl-Glu-Phe-Leu amide;

[Aib8,Glu23,Val25,Arg26,Glu34,Lys35,Aib36,Lys37]-GLP-1-(7-37)-peptidyl-Glu-Phe-Leu amide;

[Aib8,Glu23,Val25,Val29,Glu34,Lys35,Aib36,Lys37]-GLP-1-(7-37)-peptidyl-Glu-Phe-Leu amide;

[Aib8,Glu23,Val25, GI n30,Glu34,Lys35,Aib36,Lys37]-GLP-1-(7-37)-peptidyl-Glu-Phe-Leu amide;

[Aib8,Glu23,Val25,Leu33,Glu34,Lys35,Aib36,Lys37]-GLP-1-(7-37)-peptidyl-Glu-Phe-Leu amide;

[Aib8,Glu23,Val25,Lys30,Glu34,Lys35,Leu36,Lys37]-GLP-1-(7-37)-peptidyl-Glu-Phe-Leu amide;

[Aib8,Glu23,Val25,Lys30,Glu34,Lys35,Aib36,Lys37]-GLP-1-(7-37)-peptidyl-Glu-Phe-Leu amide;

[Aib8,Glu23,Val25,Gln30,His31,Glu34,Lys35,Leu36,Lys37]-GLP-1-(7-37)-peptidyl-Glu-Phe-Leu amide;

[Aib8,Glu23,Val25,Glu34,Lys35,Aib36,Leu37]-GLP-1-(7-37)-peptidyl-Ser-Phe-Leu amide;

[Aib8,Glu23,Val25,Leu33,Glu34,Lys35,Aib36,Lys37]-GLP-1-(7-37)-peptidyl-Glu-Trp-Leu amide;

[Aib8,Lys23,Val25,Glu34,Lys35,Aib36,Lys37]-GLP-1-(7-37)-peptidyl-Glu-Phe-Leu amide;

[Aib8,Glu23,Val25,Arg26,Glu34,Arg35,Val36,Arg37]-GLP-1-(7-37)-peptidyl-Glu-Phe-Leu-Lys amide;

[Gly8,Glu23,Val25,Glu34,Lys35,Val36,Lys37]-GLP-1-(7-37)-peptidyl-Glu-Phe-Leu-Gly; [Aib8,Glu23,Val25,Glu34,Lys35,Phe36,Lys37]-GLP-1-(7-37)-peptidyl-Glu-Phe-Phe amide;

[Aib8,Glu23,Val25,Gln26,Glu34,Ala35,Val36,Ala37]-GLP-1-(7-37)-peptidyl-Glu-Phe-Leu amide;

[Aib8,Glu23,Val25,Gln26,Glu34,Gln35,Aib36,Gln37]-GLP-1-(7-37)-peptidyl-Glu-Phe-Leu amide;

[Aib8,Glu23,Lys24,Val25,Glu30,His31,Glu34,Lys35,Phe36,Lys37]-GLP-1-(7-37)-peptidyl-Glu-Phe-Phe amide;

[Aib8,Glu23,Val25,Glu34,Lys35,Aib36,Lys37]-GLP-1-(7-37)-peptidyl-Glu-Phe-Leu-Glu-Lys-Val-Lys-Glu-Phe;

[Aib8,Glu23,Val25,Glu34,Lys35,Aib36,Lys37]-GLP-1-(7-37)-peptidyl-Glu-Phe-Leu-Glu-Lys-Val;

[Aib8,Glu23,Val25,Glu34,Lys35,Aib36,Lys37]-GLP-1-(7-37)-peptidyl-Glu-Phe-Leu-Glu-Lys;

[Aib8,Glu23,Val25,Glu34,Lys35,Aib36,Lys37]-GLP-1-(7-37)-peptidyl-Glu-Phe-Leu-Glu;

[Aib8,Pro22,Glu23,Val25,Glu34,Lys35,Aib36,Lys37]-GLP-1-(7-37)-peptidyl-Glu-Phe-Leu amide;

[Aib8,Val25, GI n27, GI n34,Lys35,Aib36,Lys37]-GLP-1-(7-37)-peptidyl-Gln-Phe-Leu amide;

[Lys24,Glu34,Val36,Lys37]-GLP-1-(7-37)-peptidyl-Glu-Phe-Leu amide;

[Glu34,Lys35,Val36,Lys37]-GLP-1-(7-37)-peptidyl-Glu-Phe-Leu amide;

[Glu34,Lys35,Val36,Lys37]-GLP-1-(7-37)-peptidyl-Glu-Phe-His amide;

[Glu34,Lys35,Val36,Lys37]-GLP-1-(7-37)-peptidyl-Glu-Phe-Leu-Glu amide;

[Glu34,Lys35,Val36,Lys37]-GLP-1-(7-37)-peptidyl-Glu-His-Leu amide;

[Glu34,Lys35,Val36]-GLP-1-(7-37)-peptidyl-Glu-Phe-Leu amide;

[Aib8,Glu22,Glu23,Val25,Glu34,Lys35,Val36,Lys37]-GLP-1-(7-37)-peptidyl-Glu-Leu-Leu amide;

[Aib8,Glu23,Val25,Glu34,Lys35,Val36,Lys37]-GLP-1-(7-37)-peptidyl-Glu-Lys amide;

[Gly8,Glu22,Glu23,Val25,Glu34,Lys35,Val36,Lys37]-GLP-1-(7-37)-peptidyl-Glu-Lys-Leu amide;

[Aib8,Glu23,Val25,Glu30,Glu34,Lys35,Val36,Lys37]-GLP-1-(7-37)-peptidyl-Glu-Lys-Leu amide;

[Aib8,Glu23,Val25,Glu34,Lys35,Gly36,Lys37]-GLP-1-(7-37)-peptidyl-Glu-Phe-Leu amide;

[Aib8,Glu23,Val25,Glu34,Lys35,Val36,Lys37]-GLP-1-(7-37)-peptidyl-Asp-Phe-Leu amide;

[Aib8,Glu23,Val25,Glu34,Lys35,Val36,Lys37]-GLP-1-(7-37)-peptidyl-Gly-Phe-Leu amide;

[Aib8,Glu23,Val25,Glu34,Lys35,Val36,Lys37]-GLP-1-(7-37)-peptidyl-Glu-Glu-Leu amide;

[Aib8,Glu23,Val25,Glu34,Lys35,Val36,Lys37]-GLP-1-(7-37)-peptidyl-Glu-Gly-Leu amide;

[Aib8,Glu23,Val25,Glu34,Lys35,Val36,Lys37]-GLP-1-(7-37)-peptidyl-Glu-Lys-Leu amide;

[Aib8,Glu23,Val25,Glu34,Lys35,Val36,Lys37]-GLP-1-(7-37)-peptidyl-Glu-Leu-Leu amide;

[Aib8,Glu23,Val25,Glu34,Lys35,Val36,Lys37]-GLP-1-(7-37)-peptidyl-Glu-Val-Leu amide;

[Aib8,Glu23,Val25,Glu34,Lys35,Val36,Lys37]-GLP-1-(7-37)-peptidyl-Glu-Phe-Phe amide;

[Glu34,Lys35,Val36,Lys37]-GLP-1-(7-37)-peptidyl-Glu-Phe amide;

[Glu23,Val25,Glu34,Lys35,Val36,Lys37]-GLP-1-(7-37)-peptidyl-Glu-Phe-Leu-Glu-Lys;

[Glu34,Lys35,Val36,Lys37]-GLP-1-(7-37)-peptidyl-Glu-Phe-Leu-Glu-Lys;

[Glu23,Val25,Glu34,Lys35,Val36,Lys37]-GLP-1-(7-37)-peptidyl-Glu-His-Leu-Glu-Lys;

[Glu34,Lys35,Val36,Lys37]-GLP-1-(7-37)-peptidyl-Glu-His-Leu-Glu-Lys;

[Glu34,Val36,Lys37]-GLP-1-(7-37)-peptidyl-Glu-Phe-Leu-Glu-Lys;

[Glu34,Lys35,Val36]-GLP-1-(7-37)-peptidyl-Glu-Phe-Leu-Glu-Lys;

[Glu34,Lys35,Val36,Lys37]-GLP-1-(7-37)-peptidyl-Glu-Phe-Leu-Asp-Lys;

[Glu34,Lys35,Val36,Lys37]-GLP-1-(7-37)-peptidyl-Glu-Phe-Leu-Glu-Arg;

[Glu34,Lys35,Val36,Lys37]-GLP-1-(7-37)-peptidyl-Glu-Phe-Tyr amide;

[Glu22,Glu23,Val25,Glu34,Lys35,Val36,Lys37]-GLP-1-(7-37)-peptidyl-Glu-Phe amide;

[Gly8,Glu22,Glu23,Val25,Glu34,Lys35,Val36,Lys37]-GLP-1-(7-37)-peptidyl-Glu-Phe amide;

[Aib8,Glu22,Glu23,Val25,Glu34,Lys35,Val36,Lys37]-GLP-1-(7-37)-peptidyl-Glu-Phe amide;

[Glu22,Glu23,Val25,Glu34,Lys35,Val36,Lys37]-GLP-1-(7-37)-peptidyl-Glu-Phe-Leu amide;

[Gly8,Glu22,Glu23,Val25,Glu34,Lys35,Val36,Lys37]-GLP-1-(7-37)-peptidyl-Glu-Phe-Leu amide;

[Aib8,Glu22,Glu23,Val25,Glu34,Lys35,Val36,Lys37]-GLP-1-(7-37)-peptidyl-Glu-Phe-Leu amide;

[Glu23,Val25,Arg26,Glu34,Arg35,Val36,Arg37]-GLP-1-(7-37)-peptidyl-Glu-Phe-Leu-Glu-Lys;

[Glu23,Val25,Arg26,His34,Arg35,Val36,Arg37]-GLP-1-(7-37)-peptidyl-Glu-Phe-Leu-Glu-Lys;

[Glu34,His35,Val36,Lys37]-GLP-1-(7-37)-peptidyl-Glu-Phe-Leu amide; and

[Glu34,Lys35,Val36,His37]-GLP-1-(7-37)-peptidyl-Glu-Phe-Leu amide.

Further embodiments of the present invention:

212. The GLP-1 receptor agonist according to any one of the previous embodiments, wherein said GLP-1 receptor agonist is an anti-oxidant.

213. The GLP-1 receptor agonist peptide according to any one of the previous embodiments, wherein said GLP-1 receptor agonist peptide is an anti-inflammatory agent.

214. The GLP-1 receptor agonist peptide according to any one of the previous embodiments, wherein said GLP-1 receptor agonist peptide reduces insulin resistance in diabetic patients.

215. The GLP-1 receptor agonist peptide according to any one of the previous embodiments, wherein said GLP-1 receptor agonist peptide improves HbA1C levels in diabetic patients.

216. The GLP-1 receptor agonist peptide according to any one of the previous embodiments, wherein said GLP-1 receptor agonist peptide improves HbA1C levels in diabetic patients, with 1%.

217. The GLP-1 receptor agonist according to any of the previous embodiments, wherein HbA1c is lowered in diabetes patients with at least 0.5%.

218. The GLP-1 receptor agonist according to any of the previous embodiments, wherein the terminal half-life of said peptide is prolonged.

219. The GLP-1 receptor agonist according to any of the previous embodiments, wherein the terminal half-life of said peptide in mini-pigs is at least 5 hours, at least 10 hours, at least 15 hours or at least 20 hours.

Among further embodiments of the present invention are the following:

220. A method for treating and/or preventing diseases or states associated with dyslipidemia, inflammation and vascular disorder, such as cardiovascular disease, endothelial dysfunction, hyperlipidemia, hypertriglyceridemia, hypercholesterolemia, hyperlipoproteinemia, HDL deficiency, apoA-I deficiency, coronary artery disease, atherosclerosis, hypertension, stroke, ischemia, infarction, myocardial infarction, hemorrhage, periheralperiferal vascular disease, restenosis, acute coronary syndrome, or reperfusion myocardial injury, macrovascular disorder and microvascular disorder; or treating, in an diabetes patient, a disease or state selected from cardiovascular disease, endothelial dysfunction, a macrovascular disorder, microvascular disorder, atherosclerosis and hypertension—by administering a pharmaceutically active amount of a peptide according to any one of the previous embodiments.

221. A method of treating or preventing a disease or state associated with dyslipidemia, hypercholesterolemia and inflammation, comprising administering to a patient in need thereof an effective amount of a GLP-1 receptor agonist peptide according to one of embodiments 1-219, optionally in combination with one or more additional therapeutically active compounds.

222. The method of either one of embodiments 220-221, wherein such diseases or states associated with dyslipidemia, hypercholesterolemia and inflammation, such as cardiovascular disease, endothelial dysfunction, a macrovascular disorder, microvascular disorder, diabetes, impaired glucose tolerance (IGT), atherosclerosis and hypertension.

223. A method according to any one of the embodiments 220-222, comprising administering to a patient in need thereof an effective amount of a GLP-1 receptor agonist peptide or a pharmaceutical composition according to the present invention, optionally in combination with one or more additional therapeutically active compounds.

224. A method of treating, in a diabetes patient, a disease or state selected from cardiovascular disease, endothelial dysfunction, a macrovascular disorder, microvascular disorder, atherosclerosis and hypertension, comprising administering to a diabetes patient in need thereof an effective amount of a compound according to any of embodiments 1-219, optionally in combination with one or more additional therapeutically active compounds.

225. The method according to any one of embodiments 221-224, wherein said additional therapeutically active compound is selected from antidiabetic agents, antihyperlipidemic agents, antihypertensive agents and agents for the treatment of complications resulting from, or associated with dyslipidemia, hypercholesterolemia or inflammation.

226. The method according to embodiment 221, wherein said GLP-1 receptor agonist peptide according to any of embodiments 1-219 is administered to said patient in a unit dosage form comprising from about 0.01 mg to about 1000 mg of said GLP-1 receptor agonist peptide.

227. The method according to any of embodiments 221-226, wherein said GLP-1 receptor agonist peptide according to any of embodiments 1-219 is administered to said patient, once daily.

228. The method according to any of embodiments 221-226, wherein said GLP-1 receptor agonist peptide according to any of embodiments 1-219 is administered to said patient once weekly.

229. The method according to any of embodiments 221-226, wherein said GLP-1 receptor agonist peptide according to any of embodiments 1-219 is administered parenterally, orally, nasally, buccally or sublingually.

230. The method according to any of embodiments 221-226, wherein said GLP-1 receptor agonist peptide according to any of embodiments 1-219 is administered parenterally.

Further embodiments of the invention relates to the following:

231. A pharmaceutical composition comprising a GLP-1 receptor agonist peptide according to any of embodiments 1-219.

232. The pharmaceutical composition according to embodiment 231, which further comprises a pharmaceutical acceptable carrier and/or excipient.

233. A process for preparing a pharmaceutical composition according to either of the embodiments 231-232, comprising mixing an GLP-1 receptor agonist peptide according to any of the embodiments 1-218 with pharmaceutically acceptable substances and/or excipients.

Further embodiments of the invention relates to the following:

234. A GLP-1 receptor agonist peptide according to any of embodiments 1-219, for use in therapy.

235. The GLP-1 receptor agonist peptide according to any of embodiments 1-219, for use in the treatment of diseases or states associated with dyslipidemia, hypercholesterolemia and inflammation, such as cardiovascular disease, endothelial dysfunction, macrovascular disorder, microvascular disorder, atherosclerosis and hypertension; or treating, in an diabetes patient, a disease or state selected from cardiovascular disease, endothelial dysfunction, a macrovascular disorder, microvascular disorder, atherosclerosis and hypertension.

236. The GLP-1 receptor agonist peptide according to any of embodiments 1-219, for use as a pharmaceutical in the treatment or prevention of cardiovascular disease, endothelial dysfunction, a macrovascular disorder, microvascular disorder, atherosclerosis and hypertension.

237. The GLP-1 receptor agonist peptide according to any of embodiments 1-219, for use as a medicament.

238. Use of a compound according to any of embodiments 1-219, in the manufacture of a medicament for treating and/or preventing diseases or states associated with dyslipidemia, inflammation and vascular disorder, such as cardiovascular disease, endothelial dysfunction, hyperlipidemia, hypertriglyceridemia, hypercholesterolemia, hyperlipoproteinemia, HDL deficiency, apoA-I deficiency, coronary artery disease, atherosclerosis, hypertension, stroke, ischemia, infarction, myocardial infarction, hemorrhage, periheralperiferal vascular disease, restenosis, acute coronary syndrome, or reperfusion myocardial injury, macrovascular disorder and microvascular disorder; or treating, in an diabetes patient, a disease or state selected from cardiovascular disease, endothelial dysfunction, a macrovascular disorder, microvascular disorder, atherosclerosis and hypertension.

### BRIEF DESCRIPTION OF THE DRAWINGS

The present invention is further illustrated by reference to the accompanying drawing, in which:

FIG. 1 shows the (A) acute effect of Compound 1 on blood sugar and (B) plasma concentration measured 24 h after dosing of Compound 1, when administered subcutaneously to db/db mice in different dosages (10 nmol/kg, 30 nmol/kg, 100 nmol/kg and 300 nmol/kg bw);

FIG. 2 shows the cholesterol efflux activity of Compound 1, Exendin-4 and hGLP-1;

FIG. 3 shows the plasma concentration curves after single intravenous (i.v.) or subcutaneous (s.c.) administration of Compound 1 (A) and Example 5 (B) to normal mice;

FIG. 4 shows the plasma concentration curve of Compound 1 after single intravenous (i.v.) administration to mini-pigs;

FIG. 5 shows the plasma concentration curve of Compound 4 after single intravenous (i.v.) administration to mini-pigs;

FIG. 6 shows the plasma concentration curve of Compound 5 after single intravenous (i.v.) administration to mini-pigs;

FIG. 7 shows the plasma concentration curve of Compound 6 after single intravenous (i.v.) administration to mini-pigs;

FIG. 8 shows the plasma concentration curve of Compound 7 after single intravenous (i.v.) administration to mini-pigs;

FIG. 9 shows the plasma concentration curve of Compound 8 after single intravenous (i.v.) administration to mini-pigs;

FIG. 10 shows average hydrodynamic radius (nm) from a sample of Compound 1 plotted versus incubation time (days). The incubation temperature was 37° C. and the sample concentration was 0.9 mg/mL and 45.5 mg/mL, respectively. Error bars represent standard deviations from triplicate measurements;

FIG. 11 shows average normalized scattered intensity (counts/sec) from a sample of Compound 1 plotted versus incubation time (days). The incubation temperature was 37° C. and the sample concentration was 0.9 mg/mL and 45.5 mg/mL, respectively. Error bars represent standard deviations from triplicate measurements;

FIG. 12 shows pH solubility curve for Compound 1; and

FIG. 13 shows the data from the mechanical stress assay using Thioflavin T as a fibril detection probe of Compound 1. The peptide did not show signs of fibril formation during the 45 hour experiment in any of the four solvent systems (samples A-D) which are:

Sample A: 250 μM peptide, 20 mM phosphate buffer pH 7.5;

Sample B: 250 μM peptide, 20 mM phosphate buffer pH 7.5, 25 mM m-cresol;

Sample C: 250 μM peptide, 20 mM phosphate buffer pH 7.5, 150 mM NaCl; and

Sample D: 250 μM peptide, 20 mM phosphate buffer pH 7.5, 25 mM m-cresol, 150 mM NaCl.

### EXAMPLES

The invention is further illustrated with reference to the following examples, which are not intended to be in any way limiting to the scope of the invention as claimed.

### Example 1

### Preparative Example

### Peptide Synthesis, GLP-1 Receptor Potency In Vitro, Biophysics, Cholesterol Efflux Activity In Vitro, Anti-Diabetes and Pharmakokinetics In Vivo

This experimental part starts with a list of abbreviations, and is followed by a section including general methods for synthesising and characterising peptides of the invention. Then follows a number of examples which relate to the preparation of specific GLP-1 peptides, and at the end a number of examples have been included relating to the activity and properties of these peptides.

### LIST OF ABBREVIATIONS

Aib: α-aminoisobutyric acid
API: Active Pharmaceutical Ingredient
ApoA-I: Apolipoprotein Al
AUC: Area Under the Curve
BHK: Baby Hamster Kidney
Boc: t-butyloxycarbonyl
BSA: Bovine serum albumin
CAS: Chemical Abstracts Service
Clt: 2-chlorotrityl
collidine: 2,4,6-trimethylpyridine
DCM: dichloromethane
DesH: des-amino histidine (may also be referred to as imidazopropionic acid, Imp)
DIC: diisopropylcarbodiimide
DIPEA: diisopropylethylamine
DMEM: Dulbecco's Modified Eagle's Medium (DMEM)
EDTA: ethylenediaminetetraacetic acid
EGTA: ethylene glycol tetraacetic acid
Fmoc: 9-fluorenylmethyloxycarbonyl
HATU: (O-(7-azabenzotriazol-1-yl)-1,1,3,3-tetramethyluronium hexafluorophosphate)
HBTU: (2-(1H-benzotriazol-1-yl-)-1,1,3,3 tetramethyluronium hexafluorophosphate)
HEPES: 4-(2-hydroxyethyl)-1-piperazineethanesulfonic acid
HFIP 1,1,1,3,3,3-hexafluoro-2-propanol or hexafluoroisopropanol
HOAt: 1-hydroxy-7-azabenzotriazole
HPLC: High Performance Liquid Chromatography
HSA: Human Serum Albumin
I BMX: 3-isobutyl-1-methylxanthine
Imp: Imidazopropionic acid (also referred to as des-amino histidine, DesH)
i.v. Intravenously
ivDde: 1-(4,4-dimethyl-2,6-dioxocyclohexylidene)-3-methylbutyl
LCMS: Liquid Chromatography Mass Spectroscopy
MALDI-MS: See MALDI-TOF MS
MALDI-TOF MS: Matrix-Assisted Laser Desorption/lonisation Time of Flight Mass Spectroscopy
MeOH: methanol
Mmt: 4-methoxytrityl
Mtt: 4-methyltrityl
NMP: N-methyl pyrrolidone
OEG: 8-amino-3,6-dioxaoctanic acid
OtBu: tert butyl ester
Pbf: 2,2,4,6,7-pentamethyldihydrobenzofuran-5-sulfonyl
PBS: Phosphate Buffered Saline
Pen/Strep: Penicillin/Streptomycin
RP: Reverse Phase
RP-HPLC: Reverse Phase High Performance Liquid Chromatography
RT: Room Temperature
Rt: Retention time
s.c.: Subcutaneously
SEC-HPLC: Size Exclusion High Performance Liquid Chromatography
SPA: Scintillation Proximity Assay
SPPS: Solid Phase Peptide Synthesis
tBu: tert. butyl
TFA: trifluoroacetic acid
TIS: triisopropylsilane
Tris: tris(hydroxymethyl)aminomethane or 2-amino-2-hydroxymethyl-propane-1,3-diol
UPLC: Ultra Performance Liquid Chromatography

### General Methods of Preparation

This section relates to methods for solid phase peptide synthesis (SPPS methods, including methods for de-protection of amino acids, methods for cleaving the peptide from the resin, and for its purification), as well as methods for detecting and characterising the resulting peptide (LCMS, MALDI, and UPLC methods).

The solid phase synthesis of peptides may in some cases be improved by the use of di-peptides protected on the di-peptide amide bond with a group that can be cleaved under acidic conditions such as, but not limited to, 2-Fmoc-oxy-4-methoxybenzyl, or 2,4,6-trimethoxybenzyl. In cases where a serine or a threonine is present in the peptide, pseudoproline di-peptides may be used (available from, e.g., Novabiochem, see also W. R. Sampson (1999), J. Pep. Sci. 5, 403). The Fmoc-protected amino acid derivatives used were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, or, Fmoc-Val-OH etc. supplied from e.g. Anaspec, Bachem, Iris Biotech, or Novabiochem.

Where nothing else is specified the natural L-form of the amino acids are used. The N-terminal amino acid was Boc protected at the alpha amino group (e.g. Boc-His(Boc)-OH, or Boc-His(Trt)-OH for peptides with His at the N-terminus). In case of modular albumin binding moiety attachment using SPPS the following suitably protected building blocks such as but not limited to Fmoc-8-amino-3,6-dioxaoctanoic acid, Fmoc-tranexamic acid, Fmoc-Glu-OtBu, octadecanedioic acid mono-tert-butyl ester, nonadecanedioic acid mono-tert-butyl ester, tetradecanedioic acid mono-tert-butyl ester, or 4-(9-carboxynonyloxy) benzoic acid tert-butyl ester were used. All operations stated below were performed at 250-μmol synthesis scale.

### Synthesis of Resin Bound Protected Peptide Backbone

### Method: SPPS_P

SPPS_P was performed on a Prelude Solid Phase Peptide Synthesizer from Protein Technologies (Tucson, Ariz. 85714 U.S.A.) at 250-μmol scale using six fold excess of Fmoc-amino acids (300 mM in NMP with 300 mM HOAt or Oxyma Pure®) relative to resin loading, e.g. Rinkamide-Chematrix (0.5 mmol/g) or low load Fmoc-Gly-Wang (0.35 mmol/g). Fmoc-deprotection was performed using 20% piperidine in NMP. Coupling was performed using 3:3:3:4 amino acid/(HOAt or Oxyma Pure®)/DIC/collidine in NMP. NMP and DCM top washes (7 ml, 0.5 min, 2×2 each) were performed between deprotection and coupling steps. Coupling times were generally 60 minutes. Some amino acids including, but not limited to Fmoc-Arg(Pbf)-OH, Fmoc-Aib-OH or Boc-His(Trt)-OH were “double coupled”, meaning that after the first coupling (e.g. 60 min), the resin is drained and more reagents are added (amino acid, (HOAt or Oxyma Pure®), DIC, and collidine), and the mixture allowed to react again (e.g. 60 min).

### Method: SPPS_L

SPPS_L was performed on a microwave-based Liberty peptide synthesiser from CEM Corp. (Matthews, N.C. 28106, U.S.A.) at 250-μmol or 100-μmol scale using six fold excess of Fmoc-amino acids (300 mM in NMP with 300 mM HOAt or Oxyma Pure®) relative to resin loading, e.g. Rinkamide-Chematrix (0.5 mmol/g) or low load Fmoc-Gly-Wang (0.35 mmol/g). Fmoc-deprotection was performed using 5% piperidine in NMP at up to 75° C. for 30 seconds where after the resin was drained and washed with NMP and the Fmoc-deprotection was repeated this time for 2 minutes at 75° C. Coupling was performed using 1:1:1 amino acid/(HOAt or Oxyma Pure®)/DIC in NMP. Coupling times and temperatures were generally 5 minutes at up to 75° C. Longer coupling times were used for larger scale reactions, for example 10 min. Histidine amino acids were double coupled at 50° C., or quadruple coupled if the previous amino acid was sterically hindered (e.g. Aib). Arginine amino acids were coupled at RT for 25 minutes and then heated to 75° C. for 5 min. Some amino acids such as but not limited to Aib, were “double coupled”, meaning that after the first coupling (e.g. 5 min at 75° C.), the resin is drained and more reagents are added (amino acid, (HOAt or Oxyma Pure®) and DIC), and the mixture is heated again (e.g. 5 min at 75° C.). NMP washes (5×10 ml) were performed between deprotection and coupling steps.

Clevage of Resin Bound Peptide with or without Attached Side Chains and Purification

### Method: CP_M1

After synthesis the resin was washed with DCM, and the peptide was cleaved from the resin by a 2-3 hour treatment with TFA/TIS/water (95/2.5/2.5 or 92.5/5/2.5) followed by precipitation with diethylether. The peptide was dissolved in a suitable solvent (such as, e.g., 30% acetic acid) and purified by standard RP-HPLC on a C18, 5 μM column, using acetonitrile/water/TFA. The fractions were analysed by a combination of UPLC, MALDI and LCMS methods, and the appropriate fractions were pooled and lyophilised.

### General Methods of Detection and Characterisation

LC-MS methods

### Method: LCMS 4

LCMS_{—}4 was performed on a setup consisting of Waters Acquity UPLC system and LCT Premier XE mass spectrometer from Micromass.

### Eluents:

A: 0.1% Formic acid in water

B: 0.1% Formic acid in acetonitrile

The analysis was performed at RT by injecting an appropriate volume of the sample (preferably 2-10 μl) onto the column which was eluted with a gradient of A and B. The UPLC conditions, detector settings and mass spectrometer settings were: Column: Waters Acquity UPLC BEH, C-18, 1.7 μm, 2.1 mm×50 mm. Gradient: Linear 5%-95% acetonitrile during 4.0 min (alternatively 8.0 min) at 0.4 ml/min. Detection: 214 nm (analogue output from TUV (Tunable UV detector)) MS ionisation mode: API-ES

Scan: 100-2000 amu (alternatively 500-2000 amu), step 0.1 amu.

### UPLC Methods

### Method: B4 1

The RP-analysis was performed using a Waters UPLC system fitted with a dual band detector. UV detections at 214 nm and 254 nm were collected using an ACQUITY UPLC BEH130, C18, 130 Å, 1.7 μm, 2.1 mm×150 mm column, 40° C. The UPLC system was connected to two eluent reservoirs containing: A: 99.95% H2O, 0.05% TFA; B: 99.95% CH3CN, 0.05% TFA. The following linear gradient was used: 95% A, 5% B to 5% A, 95% B over 16 minutes at a flow-rate of 0.40 ml/min.

### Compound 1

[Aib8,Glu23,Val25,Glu34,Lys35,Aib36,Lys37]-GLP-1-(7-37)-peptidyl-Glu-Phe-Leu amide

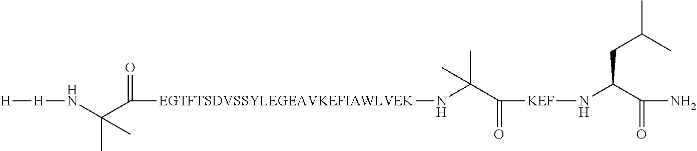

Preparation method: SPPS_P, cleavage CP_M1.

LCMS (method: LCMS_{—}4): Calc. 3859 m/z. Found: m/2: 1930, m/3: 1287, m/4: 965; Rt-uv=2.4 min.

UPLC (B4_{—}1): Rt=9.9 min.

### Compound 2

[Aib2, Glv16,Glu21,Lys29,Aib30,Leu31,Glu32,Phe33,Leu34,Glu35,Leu36,Leu371-Exendin-4-(1-37)-peptide amide

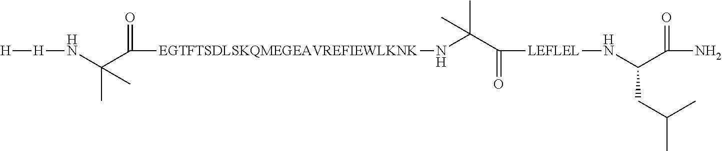

Preparation method: SPPS_P, cleavage CP_M1.

LCMS (method: LCMS_{—}4): Calc. 4338 m/z. Found: m/3: 1447, m/4: 1085, m/5: 868; Rt-uv=2.4 min.

UPLC (B4_{—}1): Rt=10.2 min.

### Compound 3

[Aib8,Glu23,Aib24,Val25,Aib30,Glu34,Lys35,Aib36,Lys37]-GLP-1-(7-37)-peptidyl-Glu-Phe-Leu amide

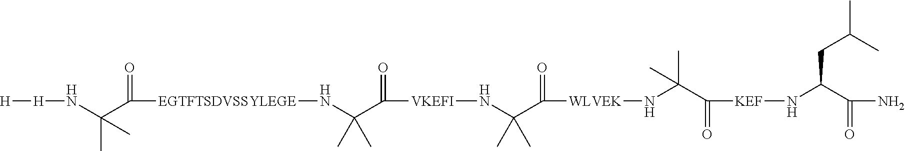

Preparation method: SPPS_P, cleavage CP_M1.

LCMS (method: LCMS_{—}4): Calc. 3887 m/z. Found: m/2: 1945, m/3: 1297, m/4: 973; Rt-uv=2.4 min.

UPLC (B4_{—}1): Rt=10.1 min.

### Compound 4

[Aib8,Glu23,Val25,Glu34,Lys35,Aib36,Lys37]-GLP-1-(7-37)-peptidyl-Glu-His-Leu amide

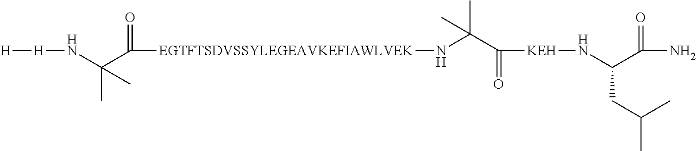

Preparation method: SPPS_P, cleavage CP_M1.

LCMS (method: LCMS_{—}4): Calc. 3849 m/z. Found: m/2: 1925, m/3: 1284, m/4: 963; Rt-uv=2.0 min.

UPLC (B4_{—}1): Rt=8.3 min.

### Compound 5

[Aib8,Glu23,Val25,Glu34,Lys35,Val36,Lys37]-GLP-1-(7-37)-peptidyl-Glu-His-Leu amide

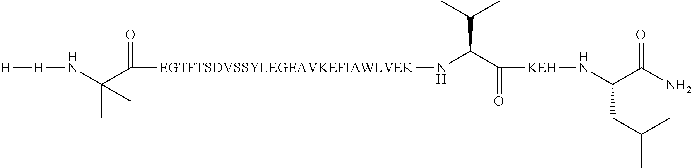

Preparation method: SPPS_P, cleavage CP_M1.

LCMS (method: LCMS_{—}4): Calc. 3863 m/z. Found: m/2: 1933, m/3: 1289, m/4: 967; Rt-uv=2.1 min.

UPLC (B4_{—}1): Rt=7.9 min.

### Compound 6

[Aib8,Glu23,Val25,Glu34,Lys35,Val36,Lys37]-GLP-1-(7-37)-peptidyl-Glu-Phe-Leu amide

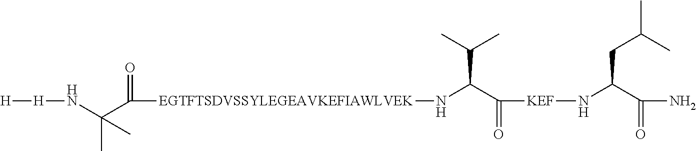

Preparation method: SPPS_P, cleavage CP_M1.

LCMS (method: LCMS_{—}4): Calc. 3873 m/z. Found: m/2: 1938, m/3: 1292, m/4: 969; Rt-uv=2.5 min.

UPLC (B4_{—}1): Rt=10.3 min.

### Compound 7

[Aib8,Val25,Glu34,Lys35,Aib36,Lys37]-GLP-1-(7-37)-peptidyl-Glu-Phe-Leu amide

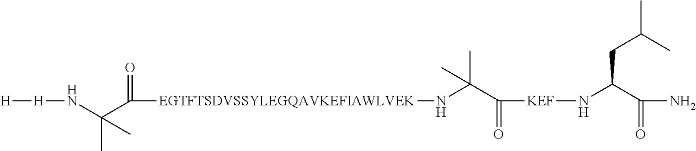

Preparation method: SPPS_P, cleavage CP_M1.

LCMS (method: LCMS_{—}4): Calc. 3858 m/z. Found: m/2: 1930, m/3: 1287, m/4: 966; Rt-uv=2.4 min.

UPLC (B4_{—}1): Rt=9.9 min.

### Compound 8

[Aib8,Glu23,Glu34,Lys35,Aib36,Lys37]-GLP-1-(7-37)-peptidyl-Glu-Phe-Leu amide

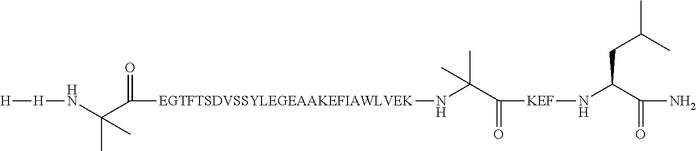

Preparation method: SPPS_P, cleavage CP_M1.

LCMS (method: LCMS_{—}4): Calc. 3831 m/z. Found: m/2: 1916, m/3: 1278, m/4: 958; Rt-uv=2.3 min.

UPLC (B4_{—}1): Rt=9.4 min.

### Compound 9

[Aib8,Glu23,Val25,Lys35,Aib36,Lys37]-GLP-1-(7-37)-peptidyl-Glu-Phe-Leu amide

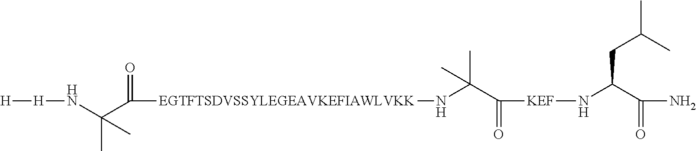

Preparation method: SPPS_P, cleavage CP_M1.

LCMS (method: LCMS_{—}4): Calc. 3858 m/z. Found: m/2: 1930, m/3: 1287, m/4: 966; Rt-uv=2.2 min.

UPLC (B4_{—}1): Rt=9.3 min.

### Compound 10

[Aib8,Glu23,Val25,Aib36,Lys371-des-Lys34-GLP-1-(7-37)-peptidyl-Glu-Phe-Leu amide

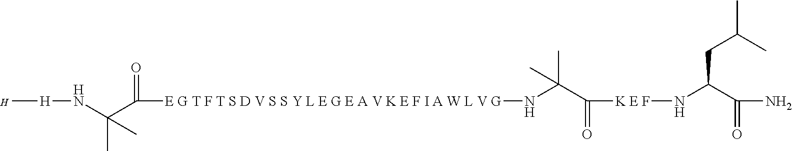

Preparation method: SPPS_P, cleavage CP_M1.

LCMS (method: LCMS_{—}4): Calc. 3659 m/z. Found: m/2: 1930, m/3: 1220, m/4: 916; Rt-uv=2.3 min.

UPLC (B4_{—}1): Rt=7.9 min.

### Compound 11

[Aib8,Glu23,Val25,Glu34,Lys35,Aib36,Lys37]-GLP-1-(7-37)-peptide amide

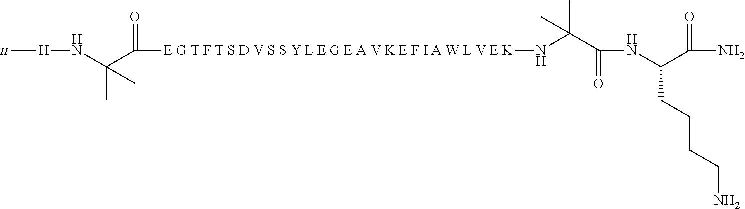

Preparation method: SPPS_P, cleavage CP_M1.

LCMS (method: LCMS_{—}4): Calc. 3470 m/z. Found: m/2: 1736, m/3: 1157, m/4: 868; Rt-uv=2.0 min.

UPLC (B4_{—}1): Rt=7.3 min.

### Compound 12

[Aib2,Gly16,Lys29,Aib30,Leu31,Glu32,Phe33,Leu34,Glu35,Leu36,Leu371-Exendin-4-(1-37)-peptide amide

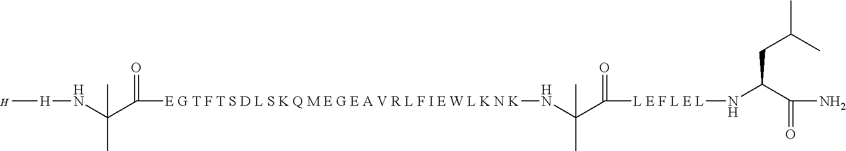

Preparation method: SPPS_P, cleavage CP_M1.

LCMS (method: LCMS_{—}4): Calc. 4322 m/z. Found: m/4: 1082, m/5: 866, m/6: 721.

UPLC (B4_{—}1): Rt=10.3 min.

### Compound 13

[Tyr12,Asn14,Thr17,Glu18,Glu23,Val25,Glu34,Lys35,Aib36,Lys37]-GLP-1-(7-37)-peptidyl-Glu-Phe-Leu amide

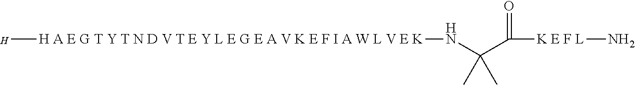

Preparation method: SPPS_P, cleavage CP_M1.

LCMS (method: LCMS_{—}4): Calc. 3944 m/z. Found: m/3: 1316, m/4: 987, m/5: 790; Rt-uv=2.5 min.

UPLC (B4_{—}1): Rt=9.8 min.

### Compound 14

[Asp9,Leu12,Ile16,Glu23,Val25,Glu34,Lys35,Aib36,Lys37]-GLP-1-(7-37)-peptidyl-Glu-Phe-Leu amide

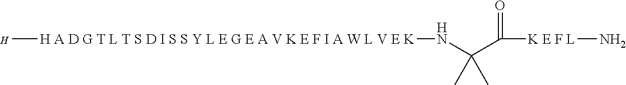

Preparation method: SPPS_P, cleavage CP_M1.

LCMS (method: LCMS_{—}4): Calc. 3811 m/z. Found: m/3: 1271, m/4: 954, m/5: 763; Rt-uv=2.5 min.

UPLC (B4_{—}1): Rt=9.9 min.

### Compound 15

[His4,Tyr20,Glu23,Val25,Glu34,Lys35,Aib36,Lys37]-GLP-1-(7-37)-peptidyl-Glu-Phe-Leu amide

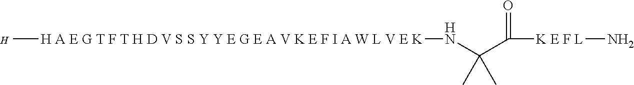

Preparation method: SPPS_P, cleavage CP_M1.

LCMS (method: LCMS_{—}4): Calc. 3945 m/z. Found: m/3: 1316, m/4: 987, m/5: 790; Rt-uv=2.4 min.

UPLC (B4_{—}1): Rt=9.3 min.

### Compound 16

[Aib8,Glu23,Val25,Glu34,Lys35,Aib36,Lys37]-GLP-1-(7-37)-peptidyl-Glu-Phe-Leu

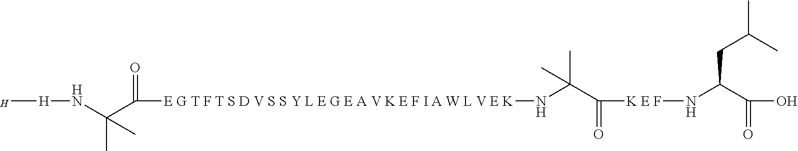

Preparation method: SPPS_P, cleavage CP_M1.

LCMS (method: LCMS_{—}4): Calc. 3860 m/z. Found: m/2: 1931, m/3: 1288, m/4: 966; Rt-uv=2.4 min.

UPLC (B4_{—}1): Rt=8.7 min.

### Compound 17

[Aib8,Glu23,Val25,His31,Glu34,Lys35,Aib36,Lys37]-GLP-1-(7-37)-peptidyl-Glu-Phe-Leu amide

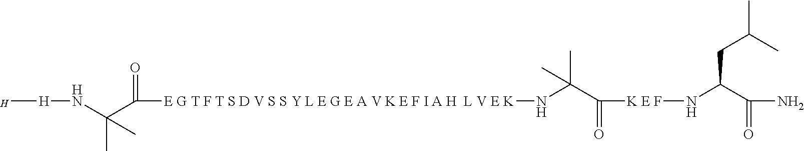

Preparation method: SPPS_P, cleavage CP_M1.

LCMS (method: LCMS_{—}4): Calc. 3810 m/z. Found: m/2: 1906, m/3: 1271, m/4: 954; Rt-uv=2.1 min.

UPLC (B4_{—}1): Rt=10.3 min.

### Compound 18

[Aib8,Asp23,Val25,Asp27,Glu34,Lys35,Aib36,Lys37]-GLP-1-(7-37)-peptidyl-Glu-Phe-Leu amide

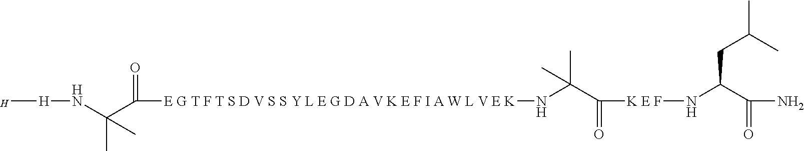

Preparation method: SPPS_P, cleavage CP_M1.

LCMS (method: LCMS_{—}4): Calc. 3831 m/z. Found: m/2: 1917, m/3: 1278, m/4: 959; Rt-uv=2.4 min.

UPLC (B4_{—}1): Rt=10.0 min.

### Compound 19

[Aib8,Glu23,Val25,Arg26,Glu34,Arg35,Aib36,Lys37]-GLP-1-(7-37)-peptidyl-Glu-Phe-Leu amide

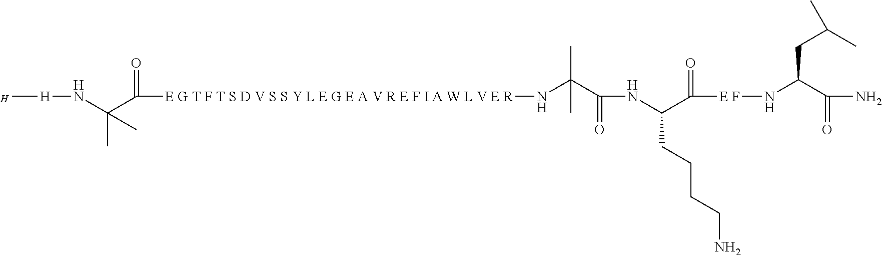

Preparation method: SPPS_P, cleavage CP_M1.

LCMS (method: LCMS_{—}4): Calc. 3915 m/z. Found: m/3: 1306, m/4: 980, m/5: 784; Rt-uv=2.1 min.

UPLC (B4_{—}1): Rt=9.4 min.

### Compound 20

[Aib8,Glu23,Val25,Glu34,Lys35,Leu36,Lys37]-GLP-1-(7-37)-peptidyl-Glu-Leu-Val amide

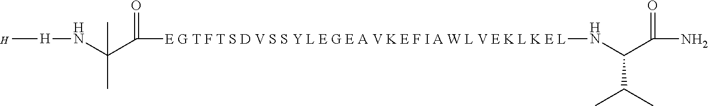

Preparation method: SPPS_P, cleavage CP_M1.

LCMS (method: LCMS_{—}4): Calc. 3839 m/z. Found: m/3: 1281, m/4: 961, m/5: 769; Rt-uv=2.2 min.

UPLC (B4_{—}1): Rt=9.8 min.

### Compound 21

[Aib8,Trp23,Val25,Glu34,Lys35,Aib36,Lys37]-GLP-1-(7-37)-peptidyl-Glu-Phe-Leu amide

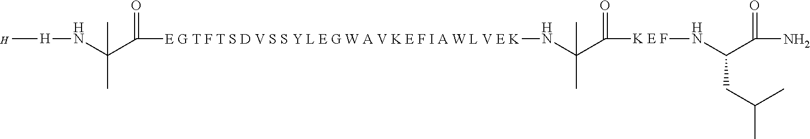

Preparation method: SPPS_P, cleavage CP_M1.

LCMS (method: LCMS_{—}4): Calc. 3917 m/z. Found: m/3: 1307, m/4: 980, m/5: 785.

UPLC (B4_{—}1): Rt=10.1 min.

### Compound 22

[Aib8,Glu23,Val25,Glu34,Lys35,Aib36,Lys37]-GLP-1-(7-37)-peptidyl-Glu-Phe-His amide

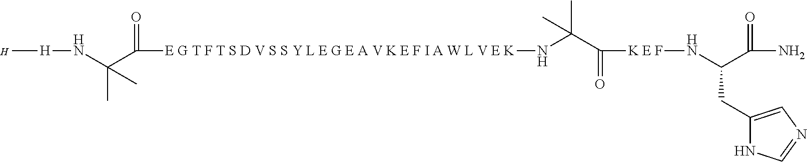

Preparation method: SPPS_P, cleavage CP_M1.

LCMS (method: LCMS_{—}4): Calc. 3883 m/z. Found: m/3: 1295, m/4: 972, m/5: 778; Rt-uv=2.0 min.

UPLC (B4_{—}1): Rt=7.9 min.

### Compound 23

[Aib2,Glu21,Lys29,Aib30,Leu31,Glu32,Phe33,Leu34,Glu35,Leu36,Leu371-Exendin-4-(1-37)-peptide

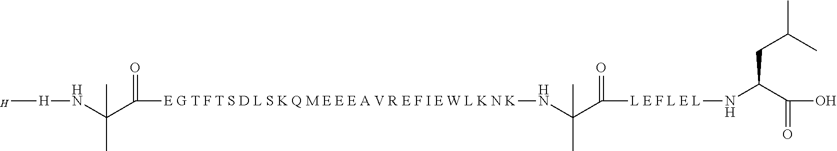

Preparation method: SPPS_P, cleavage CP_M1.

LCMS (method: LCMS_{—}4): Calc. 4411 m/z. Found: m/3: 1471, m/4: 1104, m/5: 883; Rt-uv=2.1 min.

UPLC (B4_{—}1): Rt=10.0 min.

### Compound 24

[Aib2,Gly16,Lys17,Ala18,Arg20,Glu21,Leu27,Glu28, Lvs291-Glucagonyl-(1-29)-Aib-Lys-Glu-Phe-Leu amide

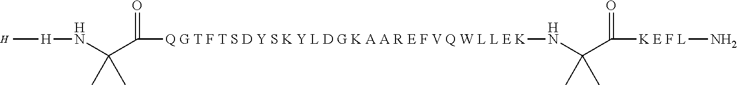

Preparation method: SPPS_P, cleavage CP_M1.

LCMS (method: LCMS_{—}4): Calc. 4005 m/z. Found: m/3: 1336, m/4: 1002, m/5: 802; Rt-uv=2.3 min.

UPLC (B4_{—}1): Rt=8.5 min.

### Compound 25

[Aib2,Gly16,Lys17,Arg20,Glu21,Leu27,Glu28,Lys291-Glucagonyl-(1-29)-Aib-Lys-Glu-Phe-Leu amide

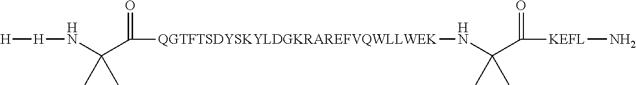

Preparation method: SPPS_P, cleavage CP_M1.

LCMS (method: LCMS_{—}4): Calc. 4090 m/z. Found: m/3: 1364, m/4: 1023, m/5: 819; Rt-uv=2.1 min.

UPLC (B4_{—}1): Rt=7.9 min.

### Compound 26

[Aib2,Lys17,Ala18,Arg20,Glu21,Leu27,Glu28,Lys291-Glucagonyl-(1-29)-Aib-Lys-Glu-Phe-Leu amide

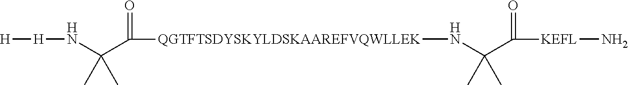

Preparation method: SPPS_P, cleavage CP_M1.

LCMS (method: LCMS_{—}4): Calc. 4035 m/z. Found: m/3: 1346, m/4: 1010, m/5: 808; Rt-uv=2.1 min.

UPLC (B4_{—}1): Rt=8.3 min.

### Compound 27

[Asn14,Met16,Thr17,Asn18,Glu23,Val25,Glu34,Lys35,Gly36,Lys37]-GLP-1-(7-37)-peptidyl-Glu-Phe-Leu amide

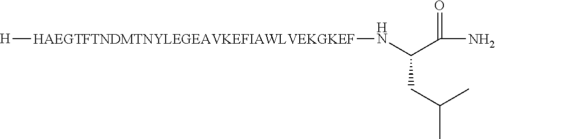

Preparation method: SPPS_P, cleavage CP_M1.

LCMS (method: LCMS_{—}4): Calc. 3917 m/z. Found: m/3: 1307, m/4: 980, m/5: 784; Rt-uv=2.3 min.

UPLC (B4_{—}1): Rt=8.7 min.

### Compound 28

[Aib8,Glu23,Val25,Leu27,Glu34,Lys35,Aib36,Lys37]-GLP-1-(7-37)-peptidyl-Glu-Phe-Leu amide

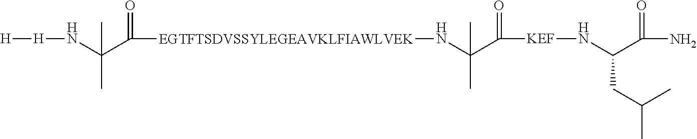

Preparation method: SPPS_P, cleavage CP_M1.

LCMS (method: LCMS_{—}4): Calc. 3843 m/z. Found: m/3: 1282, m/4: 962, m/5: 770.

UPLC (B4_{—}1): Rt=10.0 min.

### Compound 29

[Aib2,Glu21,Lys29,Aib30,Leu31,Glu32,Phe33,Leu34,Glu35,Leu36,Leu371-Exendin-4-(1-37)-peptide amide

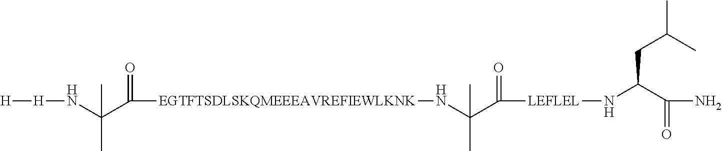

Preparation method: SPPS_P, cleavage CP_M1.

LCMS (method: LCMS_{—}4): Calc. 4410 m/z. Found: m/3: 1471, m/4: 1103, m/5: 883; Rt-uv=2.4 min.

UPLC (B4_{—}1): Rt=10.1 min.

### Compound 30

[Aib8,Glu23,Val25,Glu34,Aib36,Lys37]-GLP-1-(7-37)-peptidyl-Glu-Phe-Leu amide

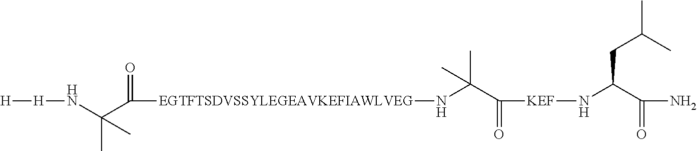

Preparation method: SPPS_P, cleavage CP_M1.

LCMS (method: LCMS_{—}4): Calc. 3788 m/z. Found: m/2: 1895, m/3: 1263, m/4: 948; Rt-uv=2.5 min.

UPLC (B4_{—}1): Rt=10.5 min.

### Compound 31

[Aib8,Glu23,Val25,Glu34,Lys35,Aib36,Lys37]-GLP-1-(7-37)-peptidyl-His-Phe-Leu amide

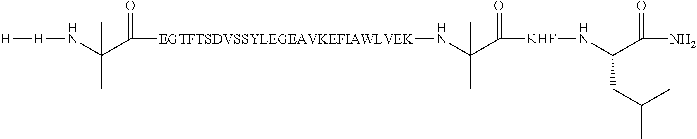

Preparation method: SPPS_P, cleavage CP_M1.

LCMS (method: LCMS_{—}4): Calc. 3867 m/z. Found: m/3: 1290, m/4: 968, m/5: 775.

UPLC (B4_{—}1): Rt=8.9 min.

### Compound 32

[Aib8,Glu23,Val25,Glu34,Lys35,Aib361-GLP-1-(7-37)-peptidyl-Glu-Phe-Leu amide

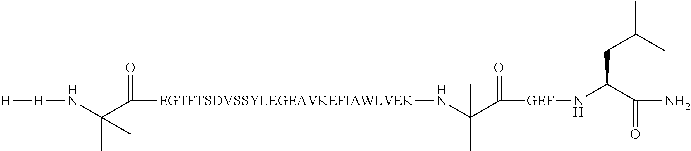

Preparation method: SPPS_P, cleavage CP_M1.

LCMS (method: LCMS_{—}4): Calc. 3788 m/z. Found: m/2: 1895, m/3: 1264, m/4: 948; Rt-uv=2.5 min.

UPLC (B4_{—}1): Rt=10.2 min.

### Compound 33

[Aib2,Glu21,Lys29,Leu31,Glu32,Phe33,Leu34,Glu35,Leu36,Leu371-Exendin-4-(1-37)-peptide amide

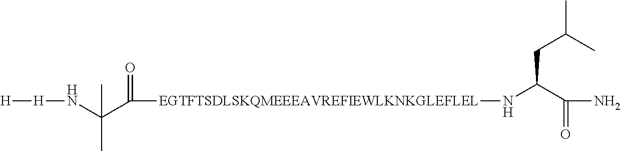

Preparation method: SPPS_P, cleavage CP_M1.

LCMS (method: LCMS_{—}4): Calc. 4382 m/z. Found: m/4: 1096, m/5: 877; Rt-uv=2.1 min.

UPLC (B4_{—}1): Rt=9.0 min.

### Compound 34

[Aib2,Glu21,Aib30,Leu31,Glu32,Phe33,Leu34,Glu35,Leu36,Leu371-Exendin-4-(1-37)-peptide amide

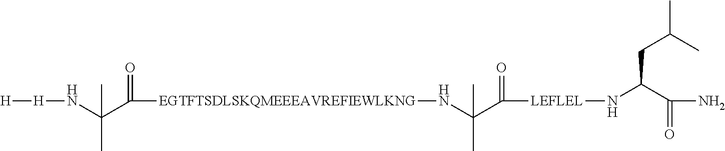

Preparation method: SPPS_P, cleavage CP_M1.

LCMS (method: LCMS_{—}4): Calc. 4339 m/z. Found: m/3: 1447, m/4: 1086, m/5: 869; Rt-uv=2.3 min.

UPLC (B4_{—}1): Rt=10.6 min.

### Compound 35

[Aib2,Glu21,Lys29,Aib30,Glu32,Phe33,Leu34,Glu35,Leu36,Leu371-Exendin-4-(1-37)-peptide amide

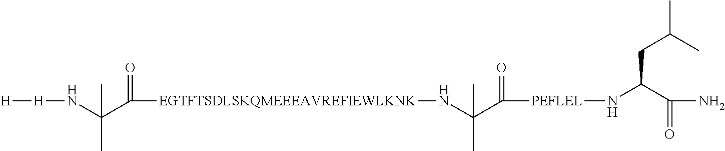

Preparation method: SPPS_P, cleavage CP_M1.

LCMS (method: LCMS_{—}4): Calc. 4394 m/z. Found: m/4: 1100, m/5: 880; Rt-uv=2.1 min.

UPLC (B4_{—}1): Rt=8.8 min.

### Compound 36

[Aib2,Glu21,Lys29,Aib30,Leu31,Phe33,Leu34,Glu35,Leu36,Leu371-Exendin-4-(1-37)-peptide amide

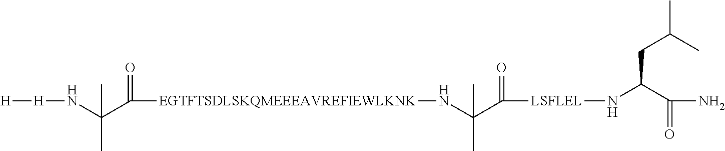

Preparation method: SPPS_P, cleavage CP_M1.

LCMS (method: LCMS_{—}4): Calc. 4368 m/z. Found: m/4: 1093, m/5: 874; Rt-uv=2.2 min.

UPLC (B4_{—}1): Rt=9.8 min.

### Compound 37

[Aib2,Glu21,Lys29,Aib30,Leu31,Glu32,Leu34,Glu35,Leu36,Leu371-Exendin-4-(1-37)-peptide amide

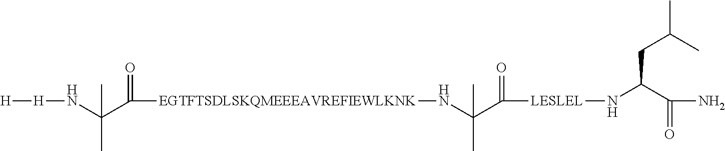

Preparation method: SPPS_P, cleavage CP_M1.

LCMS (method: LCMS_{—}4): Calc. 4350 m/z. Found: m/3: 1451, m/4: 1088, m/5: 871; Rt-uv=2.1 min.

UPLC (B4_{—}1): Rt=8.6 min.

### Compound 38

[Aib2,Glu21,Lys29,Aib30,Leu31,Glu32,Phe33,Glu35,Leu36,Leu371-Exendin-4-(1-37)-peptide amide

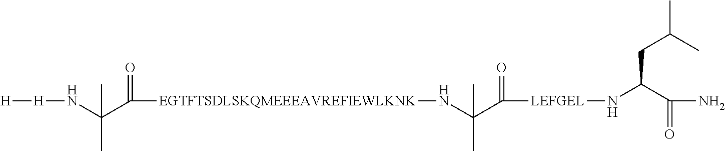

Preparation method: SPPS_P, cleavage CP_M1.

LCMS (method: LCMS_{—}4): Calc. 4354 m/z. Found: m/3: 1452, m/4: 1089, m/5: 872; Rt-uv=2.2 min.

UPLC (B4_{—}1): Rt=8.8 min.

### Compound 39

[Aib2,Glu21,Lys29,Aib30,Leu31,Glu32,Phe33,Leu34,Glu35,Leu371-Exendin-4-(1-37)-peptide amide

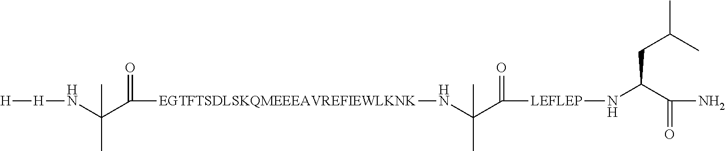

Preparation method: SPPS_P, cleavage CP_M1.

LCMS (method: LCMS_{—}4): Calc. 4394 m/z. Found: m/3: 1465, m/4: 1099, m/5: 880; Rt-uv=2.5 min.

UPLC (B4_{—}1): Rt=9.1 min.

### Compound 40

[Aib2,Glu21,Lys29,Aib30,Leu31,Glu32,Phe33,Leu34,Glu35,Leu361-Exendin-4-(1-37)-peptide amide

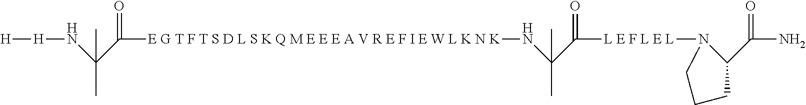

Preparation method: SPPS_P, cleavage CP_M1.

LCMS (method: LCMS_{—}4): Calc. 4394 m/z. Found: m/3: 1466, m/4: 1100, m/5: 880.

UPLC (B4_{—}1): Rt=8.8 min.

### Compound 41

[Aib2,Glu21,Lys29,Aib30,Leu31,Glu32,Phe33,Leu34,Leu36,Leu371-Exendin-4-(1-37)-peptide amide

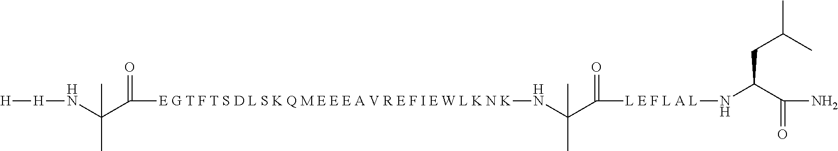

Preparation method: SPPS_P, cleavage CP_M1.

LCMS (method: LCMS_{—}4): Calc. 4352 m/z. Found: m/3: 1451, m/4: 1089, m/5: 871; Rt-uv=2.4 min.

UPLC (B4_{—}1): Rt=10.2 min.

### Compound 42

[Aib2,Glu21, Lvs29,Aib30,Leu31,Glu32,Phe33,Leu34]-Exendin-4-(1-34)-peptide amide

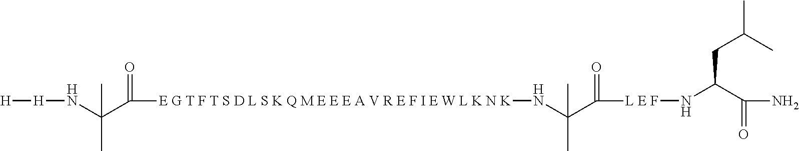

Preparation method: SPPS_P, cleavage CP_M1.

LCMS (method: LCMS_{—}4): Calc. 4055 m/z. Found: m/3: 1352, m/4: 1015, m/5: 812; Rt-uv=2.1 min.

UPLC (B4_{—}1): Rt=8.7 min.

### Compound 43

[Aib8,Glu23,Val25,Arg26,Glu34,Arg35,Aib36,Arg371-GLP-1-(7-37)-peptidyl-Glu-Phe-Leu amide

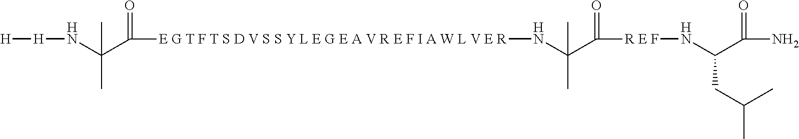

Preparation method: SPPS_P, cleavage CP_M1.

LCMS (method: LCMS_{—}4): Calc. 3943 m/z. Found: m/3: 1315, m/4: 987, m/5: 790; Rt-uv=2.3 min.

UPLC (B4_{—}1): Rt=9.6 min.

### Compound 44

[Asn14,Met16,Thr17,Asn18,Glu23,Val25,Glu34,Lys35,Aib36,Lys37]-GLP-1-(7-37)-peptidyl-Glu-Phe-Leu amide

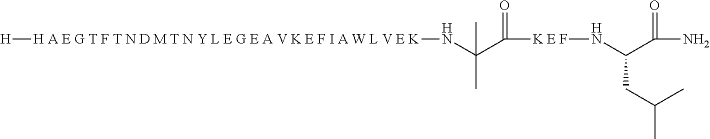

Preparation method: SPPS_P, cleavage CP_M1.

LCMS (method: LCMS_{—}4): Calc. 3945 m/z. Found: m/3: 1316, m/4: 987, m/5: 790; Rt-uv=2.3 min.

UPLC (B4_{—}1): Rt=9.9 min.

### Compound 45

[Aib8,Glu34,Lys35,Aib36,Lys37]-GLP-1-(7-37)-peptidyl-Glu-Phe-Leu amide

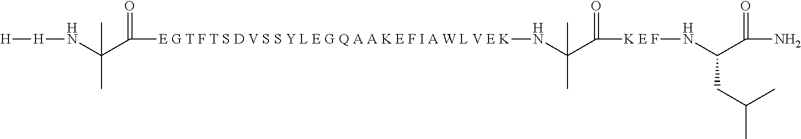

Preparation method: SPPS_P, cleavage CP_M1.

LCMS (method: LCMS_{—}4): Calc. 3830 m/z. Found: m/2: 1916, m/3: 1278, m/4: 958; Rt-uv=2.3 min.

UPLC (B4_{—}1): Rt=9.4 min.

### Compound 46

[Aib8,Glu23,Val25,Glu34,Lys35,Phe36,Lys37]-GLP-1-(7-37)-peptidyl-Glu-Ala-Phe amide

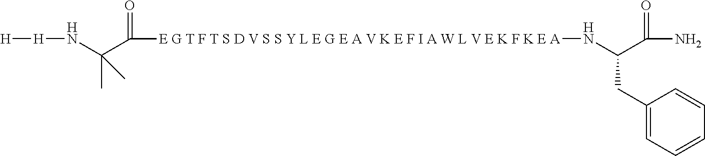

Preparation method: SPPS_P, cleavage CP_M1.

LCMS (method: LCMS_{—}4): Calc. 3879 m/z. Found: m/2: 1941, m/3: 1294, m/4: 971; Rt-uv=2.3 min.

UPLC (B4_{—}1): Rt=9.4 min.

### Compound 47

[Aib8,Lys35,Aib36,Lys37]-GLP-1-(7-37)-peptidyl-Glu-Phe-Leu amide

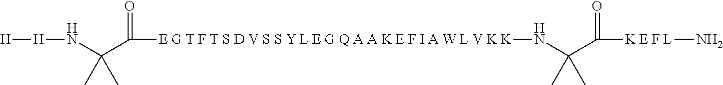

Preparation method: SPPS_P, cleavage CP_M1.

LCMS (method: LCMS_{—}4): Calc. 3829 m/z. Found: m/3: 1277, m/4: 958, m/5: 767; Rt-uv=1.8 min.

UPLC (B4_{—}1): Rt=8.9 min.

### Compound 48

[Aib8,Lys35,Aib361-GLP-1-(7-37)-peptidyl-Glu-Phe-Leu amide

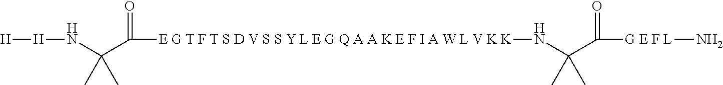

Preparation method: SPPS_P, cleavage CP_M1.

LCMS (method: LCMS_{—}4): Calc. 3758 m/z. Found: m/3: 1254, m/4: 941, m/5: 753; Rt-uv=1.8 min.

UPLC (B4_{—}1): Rt=9.2 min.

### Compound 49

[Aib8,Glu23,Lys24,Val25,Glu30,Glu34,Lys35,Aib36,Lys37]-GLP-1-(7-37)-peptidyl-Glu-Phe-Leu amide

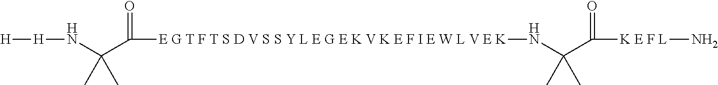

Preparation method: SPPS_P, cleavage CP_M1.

LCMS (method: LCMS_{—}4): Calc. 3975 m/z. Found: m/3: 1326, m/4: 995, m/5: 796; Rt-uv=1.8 min.

UPLC (B4_{—}1): Rt=9.2 min.

### Compound 50

[Aib8,Glu23,Val25,Trp30,Glu34,Lys35,Aib36,Lys37]-GLP-1-(7-37)-peptidyl-Glu-Phe-Leu amide

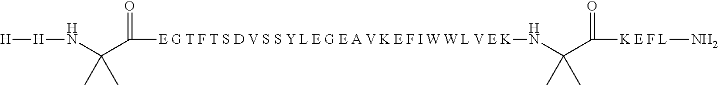

Preparation method: SPPS_P, cleavage CP_M1.

LCMS (method: LCMS_{—}4): Calc. 3975 m/z. Found: m/3: 1326, m/4: 995, m/5: 796; Rt-uv=1.9 min.

UPLC (B4_{—}1): Rt=10.0 min.

### Compound 51

[Aib8,Glu23,Val25,Lys27,Glu34,Lys35,Aib36,Lys37]-GLP-1-(7-37)-peptidyl-Glu-Phe-Leu amide

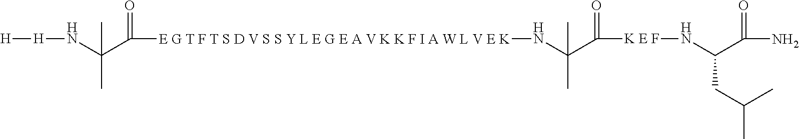

Preparation method: SPPS_P, cleavage CP_M1.

LCMS (method: LCMS_{—}4): Calc. 3858 m/z. Found: m/3: 1287, m/4: 966, m/5: 773; Rt-uv=2.2 min.

UPLC (B4_{—}1): Rt=9.4 min.

### Compound 52

[Aib8,Lys23,Arq24,Arq26,Glu34,Lys35,Aib36,Lys37]-GLP-1-(7-37)-peptidyl-Glu-Phe-Leu amide

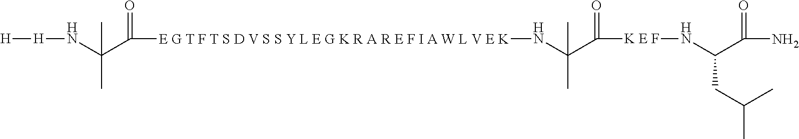

Preparation method: SPPS_P, cleavage CP_M1.

LCMS (method: LCMS_{—}4): Calc. 3944 m/z. Found: m/3: 1316, m/4: 987, m/5: 790; Rt-uv=2.0 min.

UPLC (B4_{—}1): Rt=8.3 min.

### Compound 53

[Aib8,Glu23,Arg24,Val25,Glu34,Lys35,Aib36,Lys37]-GLP-1-(7-37)-peptidyl-Glu-Phe-Leu amide

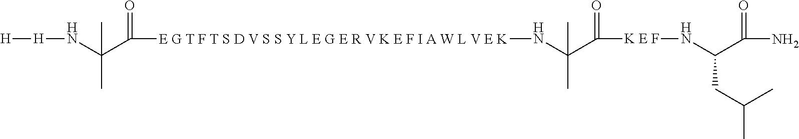

Preparation method: SPPS_P, cleavage CP_M1.

LCMS (method: LCMS_{—}4): Calc. 3945 m/z. Found: m/3: 1316, m/4: 987, m/5: 790; Rt-uv=2.2 min.

UPLC (B4_{—}1): Rt=9.0 min.

### Compound 54

[Aib8,Glu23,Val25,Glu34,Lys35,Aib36,Lys37]-GLP-1-(7-37)-peptidyl-Glu-Phe-Leu-Glu-Lys-Val amide

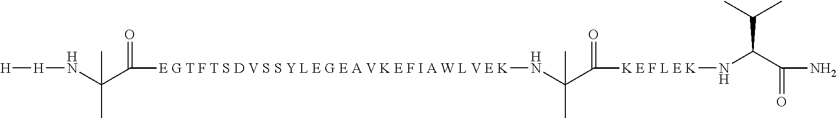

Preparation method: SPPS_P, cleavage CP_M1.

LCMS (method: LCMS_{—}4): Calc. 4216 m/z. Found: m/3: 1407, m/4: 1055, m/5: 844.

UPLC (B4_{—}1): Rt=9.9 min.

### Compound 55

[Aib2,His3,Glu15,Glu16,Glu17,Ala18,Lys20,Glu21,Ile23,Ala24,Leu27,Glu28,Lys291-Glucagonyl-(1-29)-Aib-Lys-Glu-Phe-Leu amide

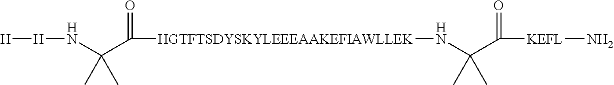

Preparation method: SPPS_P, cleavage CP_M1.

LCMS (method: LCMS_{—}4): Calc. 4031 m/z. Found: m/3: 1344, m/4: 1009, m/5: 807; Rt-uv=2.1 min.

UPLC (B4_{—}1): Rt=8.7 min.

### Compound 56

[Glu15,Glu16,Gln17,Ala18,Lys20,Glu21,Ile23,Ala24,Leu27,Glu28,Lys291-Glucagonyl-(1-29)-Aib-Lys-Glu-Phe-Leu amide

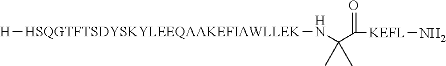

Preparation method: SPPS_P, cleavage CP_M1.

LCMS (method: LCMS_{—}4): Calc. 4023 m/z. Found: m/3: 1342, m/4: 1007, m/5: 805; Rt-uv=2.1 min.

UPLC (B4_{—}1): Rt=8.8 min.

### Compound 57

[Glu15,Glu16,Lys17,Ala18,Lys20,Glu21,Ile23,Ala24,Leu27,Glu28,Lys291-Glucagonyl-(1-29)-Aib-Lys-Glu-Phe-Leu amide

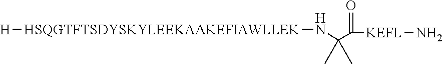

Preparation method: SPPS_P, cleavage CP_M1.

LCMS (method: LCMS_{—}4): Calc. 4023 m/z. Found: m/3: 1342, m/4: 1007, m/5: 805; Rt-uv=2.1 min.

UPLC (B4_{—}1): Rt=8.5 min.

### Compound 58

[Glu15,Glu16,Lys17,Lys18,Lys20,Glu21,Ile23,Ala24,Leu27,Glu28,Lys291-Glucagonyl-(1-29)-Aib-Lys-Glu-Phe-Leu amide

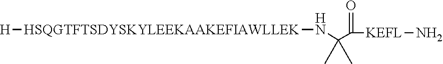

Preparation method: SPPS_P, cleavage CP_M1.

LCMS (method: LCMS_{—}4): Calc. 4080 m/z. Found: m/3: 1361, m/4: 1021, m/5: 817; Rt-uv=1.9 min.

UPLC (B4_{—}1): Rt=7.9 min.

### Compound 59

[Lys17,Ala18,Arg20,Glu21,Leu27,Glu28,Lys291-Glucagonyl-(1-29)-Aib-Lys-Glu-Phe-Leu amide

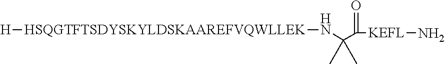

Preparation method: SPPS_P, cleavage CP_M1.

LCMS (method: LCMS_{—}4): Calc. 4038 m/z. Found: m/3: 1347, m/4: 1010, m/5: 808; Rt-uv=2.0 min.

UPLC (B4_{—}1): Rt=8.3 min.

### Compound 60

[Aib8,Glu23,Lys24,Val25,Glu34,Lys35,Aib36,Lys37]-GLP-1-(7-37)-peptidyl-Glu-Phe-Leu amide

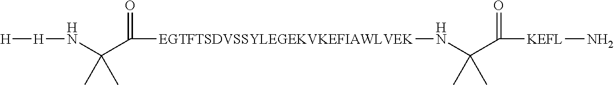

Preparation method: SPPS_P, cleavage CP_M1.

LCMS (method: LCMS_{—}4): Calc. 3917 m/z. Found: m/3: 1306, m/4: 980, m/5: 784; Rt-uv=2.2 min.

UPLC (B4_{—}1): Rt=9.1 min.

### Compound 61

[Aib8,Glu23,Val25,Glu34,Lys35,Aib36,Lys37]-GLP-1-(7-37)-peptidyl-Glu-Phe-Leu-Glu-Lys-Val-Lys-Glu-Phe amide

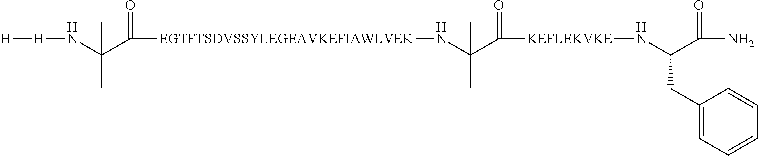

Preparation method: SPPS_P, cleavage CP_M1.

LCMS (method: LCMS_{—}4): Calc. 4620 m/z. Found: m/3: 1542, m/4: 1156, m/5: 925; Rt-uv=2.4 min.

UPLC (B4_{—}1): Rt=9.8 min.

### Compound 62

[Aib8,Glu23,Val25,Glu34,Lys35,Aib36,Lys37]-GLP-1-(7-37)-peptidyl-Glu-Phe amide

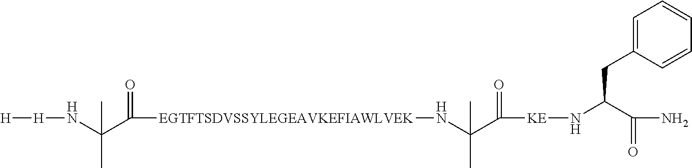

Preparation method: SPPS_P, cleavage CP_M1.

LCMS (method: LCMS_{—}4): Calc. 3746 m/z. Found: m/3: 1250, m/4: 938, m/5 750; Rt-uv=2.9 min.

UPLC (B4_{—}1): Rt=8.5 min.

### Compound 63

[Aib8,Glu23,Val25,Glu34,Lys35,Aib36,Lys37]-GLP-1-(7-37)-peptidyl-Glu amide

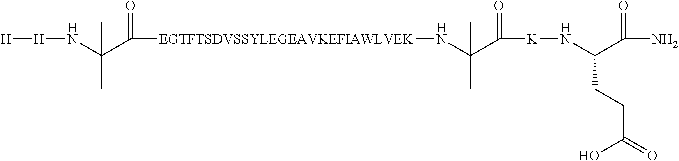

Preparation method: SPPS_P, cleavage CP_M1.

LCMS (method: LCMS_{—}4): Calc. 3599 m/z. Found: m/3: 1200, m/4: 900; Rt-uv=2.1 min.

UPLC (B4_{—}1): Rt=7.8 min.

### Compound 64

[Aib8,Glu23,Lys24,Val25,Glu34,Aib36,Lys37]-GLP-1-(7-37)-peptidyl-Glu-Phe-Leu amide

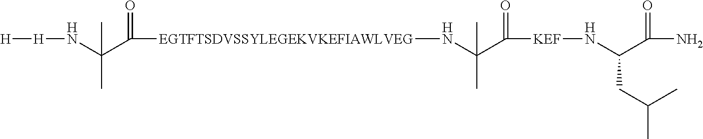

Preparation method: SPPS_P, cleavage CP_M1.

LCMS (method: LCMS_{—}4): Calc. 3845 m/z. Found: m/3: 1283, m/4: 962; Rt-uv=2.4 min.

UPLC (B4_{—}1): Rt=9.2 min.

### Compound 65

[Aib8,Glu23,Val25,Val29,Gln30,Glu34,Lys35,Aib36,Lys37]-GLP-1-(7-37)-peptidyl-Glu-Phe-Leu amide

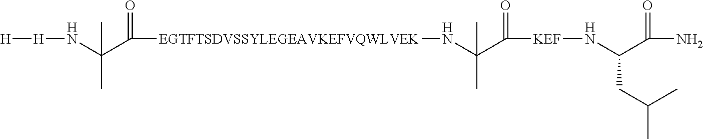

Preparation method: SPPS_P, cleavage CP_M1.

LCMS (method: LCMS_{—}4): Calc. 3902 m/z. Found: m/3: 1302, m/4: 976; Rt-uv=2.4 min.

UPLC (B4_{—}1): Rt=9.5 min.

### Compound 66

[Aib8,Glu23,Val25,Glu34,Lys35,Aib36,Lys37]-GLP-1-(7-37)-peptidyl-Glu-Phe-Leu-Glu amide

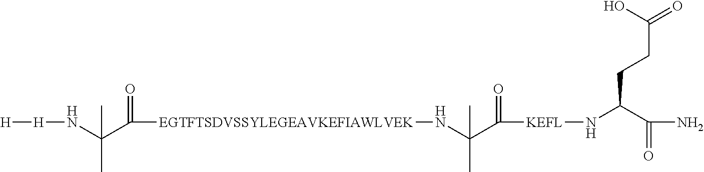

Preparation method: SPPS_P, cleavage CP_M1.

LCMS (method: LCMS_{—}4): Calc. 3989 m/z. Found: m/3: 1331, m/4: 997; Rt-uv=3.1 min.

UPLC (B4_{—}1): Rt=9.8 min.

### Compound 67

[Glu23,Val25,Glu34,Lys35,Val36,Lys37]-GLP-1-(7-37)-peptidyl-Glu-Phe-Leu amide

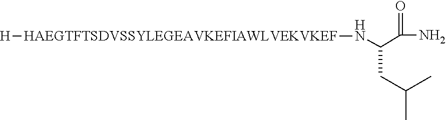

Preparation method: SPPS_P, cleavage CP_M1.

LCMS (method: LCMS_{—}4): Calc. 3859 m/z. Found: m/3: 1278, m/4: 959, m/5: 767; Rt-uv=3.0 min.

UPLC (B4_{—}1): Rt=9.5 min.

### Compound 68

[Ala2,Lys17,Ala18,Arg20,Glu21,Leu27,Glu28,Lys291-Glucagonyl-(1-29)-Val-Lys-Glu-Phe-Leu amide

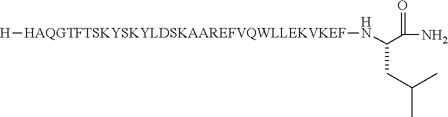

Preparation method: SPPS_P, cleavage CP_M1.

LCMS (method: LCMS_{—}4): Calc. 4036 m/z. Found: m/4. 1010, m/5: 808; Rt-uv=2.8 min.

UPLC (B4_{—}1): Rt=8.3 min.

### Compound 69

[Ala2,Glu21,Lys29,Val30,Leu31,Glu32,Phe33,Leu34]-Exendin-4-(1-34)-peptide amide

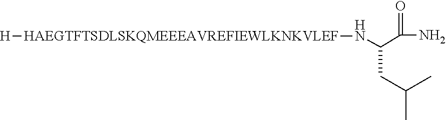

Preparation method: SPPS_P, cleavage CP_M1.

LCMS (method: LCMS_{—}4): Calc. 4055 m/z. Found: m/3: 1352, m/4:1015; m/5 812, Rt-uv=2.2 min.

UPLC (B4_{—}1): Rt=8.8 min.

### Compound 70

[Aib8,Glu23,Val25,Glu34,Lys35,Aib36,Lys37]-GLP-1-(7-37)-peptidyl-Glu-Phe-Leu-Glu-Lys amide

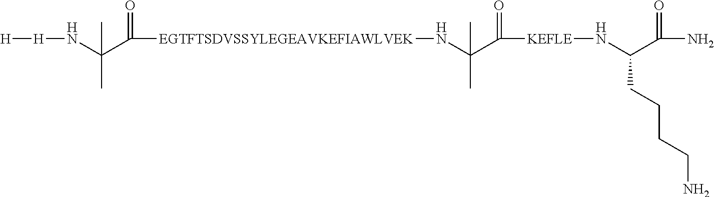

Preparation method: SPPS_P, cleavage CP_M1.

LCMS (method: LCMS_{—}4): Calc. 4117 m/z. Found: m/3: 1373, m/4: 1030, m/5 824; Rt-uv=2.4 min.

UPLC (B4_{—}1): Rt=9.2 min.

### Compound 71

[Aib8,Glu23,Lys24,Val25,Glu30,His31,Glu34,Lys35,Aib36,Lys37]-GLP-1-(7-37)-peptidyl-Glu-Phe-Leu amide

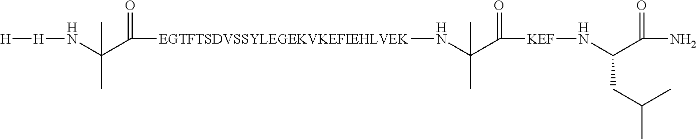

Preparation method: SPPS_P, cleavage CP_M1.

LCMS (method: LCMS_{—}4): Calc. 3925 m/z. Found: m/3: 1310, m/4: 982; m/5. 786; Rt-uv=2.7 min.

UPLC (B4_{—}1): Rt=7.9 min.

### Compound 72

[Aib8,Val25,Gln27,Glu34,Lys35,Aib36,Ala371-GLP-1-(7-37)-peptidyl-Glu-Phe-Leu amide

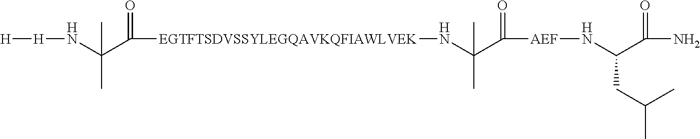

Preparation method: SPPS_P, cleavage CP_M1.

LCMS (method: LCMS_{—}4): Calc. 3800 m/z. Found: m/3: 1268, m/4: 951.

UPLC (B4_{—}1): Rt=10.6 min.

### Compound 73

[Aib8,Val25,Asn34,Lys35,Aib36,Ala371-GLP-1-(7-37)-peptidyl-Glu-Phe-Leu amide

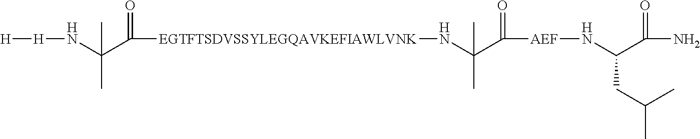

Preparation method: SPPS_P, cleavage CP_M1.

LCMS (method: LCMS_{—}4): Calc. 3786 m/z. Found: m/3: 1263, m/4: 948.

UPLC (B4_{—}1): Rt=10.5 min.

### Compound 74

[Aib8,Val25,Gln27,Asn34,Lys35,Aib36,Ala371-GLP-1-(7-37)-peptidyl-Glu-Phe-Leu amide

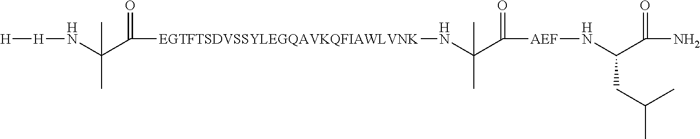

Preparation method: SPPS_P, cleavage CP_M1.

LCMS (method: LCMS_{—}4): Calc. 3785 m/z. Found: m/3: 1263, m/4: 948.

UPLC (B4_{—}1): Rt=10.4 min.

### Compound 75

[Aib8,Val361-GLP-1-(7-37)-peptidyl-Glu-Phe-Leu amide

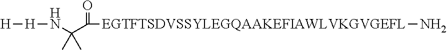

Preparation method: SPPS_P, cleavage CP_M1.

LCMS (method: LCMS_{—}4): Calc. 3701 m/z. Found: m/2: 1851, m/3: 1235, m/4: 926; Rt-uv=2.9 min.

UPLC (B4_{—}1): Rt=9.6 min.

### Compound 76

[Gly8,Glu23,Val25,Glu34,Lys35,Val36,Lys37]-GLP-1-(7-37)-peptidyl-Glu-Phe-Leu amide

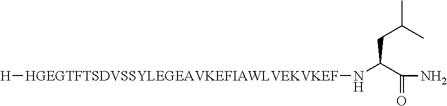

Preparation method: SPPS_P, cleavage CP_M1.

LCMS (method: LCMS_{—}4): Calc. 3845 m/z. Found: m/3: 1283, m/4: 962, m/5: 770; Rt-uv=3.1 min.

UPLC (B4_{—}1): Rt=10.1 min.

### Compound 77

[desamino-His7,Aib8,Glu23,Val25,Glu34,Lys35,Val36,Lys37]-GLP-1-(7-37)-peptidyl-Glu-Phe-Leu amide

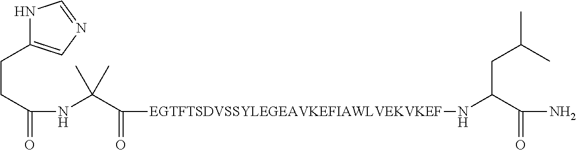

Preparation method: SPPS_P, cleavage CP_M1.

LCMS (method: LCMS_{—}4): Calc. 3858 m/z. Found: m/3: 1287, m/4: 965; Rt-uv=3.2 min.

UPLC (B4_{—}1): Rt=10.6 min.

### Compound 78

[Gly8,Glu23,Val25,Glu34,Lys35,Val36,Lys37]-GLP-1-(7-37)-peptidyl-Glu-His-Leu amide

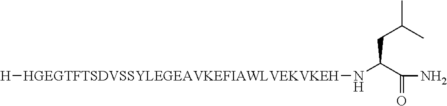

Preparation method: SPPS_P, cleavage CP_M1.

LCMS (method: LCMS_{—}4): Calc. 3835 m/z. Found: m/3: 1279, m/4: 960, m/5: 768; Rt-uv=2.9 min.

UPLC (B4_{—}1): Rt=8.2 min.

### Compound 79

[Aib8,Glu23,Val25,Gln30,Leu33,Glu34,Lys35,Aib36,Lys37]-GLP-1-(7-37)-peptidyl-Glu-Phe-Leu amide

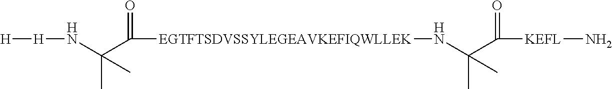

Preparation method: SPPS_P, cleavage CP_M1.

LCMS (method: LCMS_{—}4): Calc. 3930 m/z. Found: m/3: 1311, m/4: 983, m/5: 787; Rt-uv=2.6 min.

UPLC (B4_{—}1): Rt=10.0 min.

### Compound 80

[Aib8,Glu23,Val25,Arq26,Val29,Gln30,Leu33,Glu34,Lys35,Aib36,Lys37]-GLP-1-(7-37)-peptidyl-Glu-Phe-Leu amide

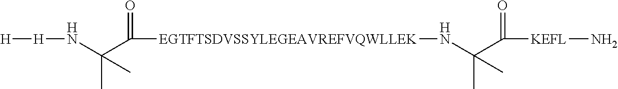

Preparation method: SPPS_P, cleavage CP_M1.

LCMS (method: LCMS_{—}4): Calc. 3944 m/z. Found: m/3: 1316, m/4: 987, m/5: 790; Rt-uv=2.6 min.

UPLC (B4_{—}1): Rt=9.8 min.

### Compound 81

[Aib8,Glu23,Val25,Arg26,Glu34, Lvs35,Aib36, Lvs371-GLP-1-(7-37)-peptidyl-Glu-Phe-Leu amide

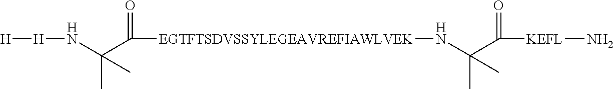

Preparation method: SPPS_P, cleavage CP_M1.

LCMS (method: LCMS_{—}4): Calc. 3887 m/z. Found: m/3: 1297, m/4: 973, m/5: 778; Rt-uv=2.6 min.

UPLC (B4_{—}1): Rt=9.9 min.

### Compound 82

[Aib8,Glu23,Val25,Val29,Glu34,Lys35,Aib36,Lys37]-GLP-1-(7-37)-peptidyl-Glu-Phe-Leu amide

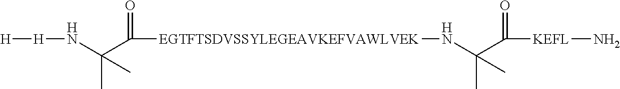

Preparation method: SPPS_P, cleavage CP_M1.

LCMS (method: LCMS_{—}4): Calc. 3845 m/z. Found: m/3: 1283, m/4: 962, m/5: 770; Rt-uv=3.0 min.

UPLC (B4_{—}1): Rt=9.6 min.

### Compound 83

[Aib8,Glu23,Val25,Gln30,Glu34,Lys35,Aib36,Lys37]-GLP-1-(7-37)-peptidyl-Glu-Phe-Leu amide

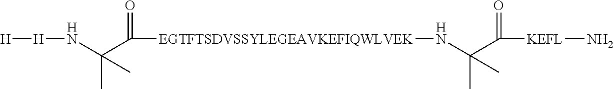

Preparation method: SPPS_P, cleavage CP_M1.

LCMS (method: LCMS_{—}4): Calc. 3916 m/z. Found: m/3: 1306, m/4: 980, m/5: 784; Rt-uv=3.0 min.

UPLC (B4_{—}1): Rt=9.8 min.

### Compound 84

[Aib8,Glu23,Val25,Leu33,Glu34,Lys35,Aib36,Lys37]-GLP-1-(7-37)-peptidyl-Glu-Phe-Leu amide

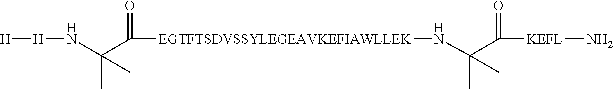

Preparation method: SPPS_P, cleavage CP_M1.

LCMS (method: LCMS_{—}4): Calc. 3873 m/z. Found: m/3: 1292, m/4: 969, m/5: 776; Rt-uv=3.1 min.

UPLC (B4_{—}1): Rt=10.0 min.

### Compound 85

[Aib8,Glu23,Val25,Lys30,Glu34,Lys35,Leu36,Lys37]-GLP-1-(7-37)-peptidyl-Glu-Phe-Leu amide

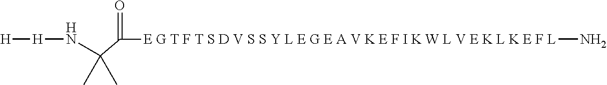

Preparation method: SPPS_P, cleavage CP_M1.

LCMS (method: LCMS_{—}4): Calc. 3944 m/z. Found: m/3: 1316, m/4: 987; Rt-uv=3.0 min.

UPLC (B4_{—}1): Rt=9.8 min.

### Compound 86

[Aib8,Glu23,Val25,Lys30,Glu34,Lys35,Aib36,Lys37]-GLP-1-(7-37)-peptidyl-Glu-Phe-Leu amide

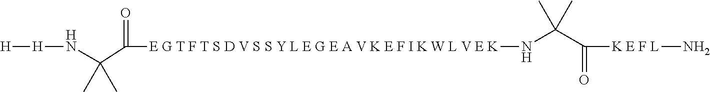

Preparation method: SPPS_P, cleavage CP_M1.

LCMS (method: LCMS_{—}4): Calc. 3916 m/z. Found: m/3: 1306, m/4: 980; Rt-uv=3.0 min.

UPLC (B4_{—}1): Rt=9.4 min.

### Compound 87

[Aib8,Glu23,Val25,Gln30,His31,Glu34,Lys35,Leu36,Lys37]-GLP-1-(7-37)-peptidyl-Glu-Phe-Leu amide

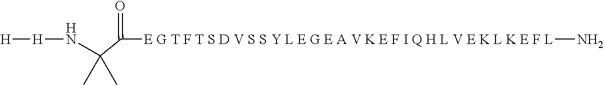

Preparation method: SPPS_P, cleavage CP_M1.

LCMS (method: LCMS_{—}4): Calc. 3895 m/z. Found: m/3: 1299, m/4: 975; Rt-uv=2.9 min.

UPLC (B4_{—}1): Rt=8.9 min.

### Compound 88

[Aib8,Glu23,Val25,Glu34,Lys35,Aib36,Leu371-GLP-1-(7-37)-peptidyl-Ser-Phe-Leu amide

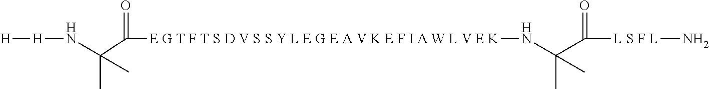

Preparation method: SPPS_P, cleavage CP_M1.

LCMS (method: LCMS_{—}4): Calc. 3802 m/z. Found: m/2: 1902, m/3: 1268, m/4: 951, m/5: 761; Rt-uv=3.1 min.

UPLC (B4_{—}1): Rt=11.3 min.

### Compound 89

[Aib8,Glu23,Val25,Leu33,Glu34,Lys35,Aib36,Lys37]-GLP-1-(7-37)-peptidyl-Glu-Trp-Leu amide

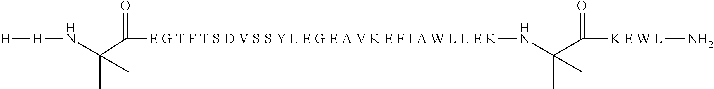

Preparation method: SPPS_P, cleavage CP_M1.

LCMS (method: LCMS_{—}4): Calc. 3912 m/z. Found: m/3: 1305, m/4: 979, m/5: 783; Rt-uv=2.8 min.

UPLC (B4_{—}1): Rt=9.9 min.

### Compound 90

[Aib8,Lys23,Val25,Glu34,Lys35,Aib36,Lys37]-GLP-1-(7-37)-peptidyl-Glu-Phe-Leu amide

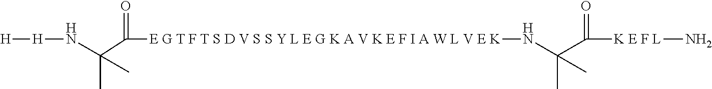

Preparation method: SPPS_P, cleavage CP_M1.

LCMS (method: LCMS_{—}4): Calc. 3858 m/z. Found: m/3: 1287, m/4: 966, m/5: 773; Rt-uv=2.7 min.

UPLC (B4_{—}1): Rt=9.4 min.

### Compound 91

[Aib8,Glu23,Val25,Arg26,Glu34,Arg35,Val36,Arg371-GLP-1-(7-37)-peptidyl-Glu-Phe-Leu-Lys amide

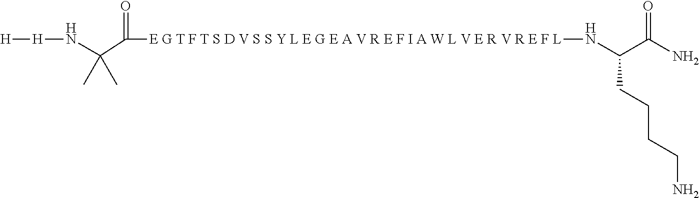

Preparation method: SPPS_P, cleavage CP_M1.

LCMS (method: LCMS_{—}4): Calc. 4086 m/z. Found: m/3: 1368, m/4: 1022, m/5: 818; Rt-uv=2.7 min.

UPLC (B4_{—}1): Rt=9.4 min.

### Compound 92

[Gly8,Glu23,Val25,Glu34,Lys35,Val36,Lys37]-GLP-1-(7-37)-peptidyl-Glu-Phe-Leu-Gly

### ^{H}-HGEGTFTSDVSSYLEGEAVKEFIAWLVEKVKEFLG-^{CH}

To express Compound 92, a pET11 based expression construct was made that expresses a fusion peptide consisting of hleptin fused to the N-terminal of Compound 92 through a linker containing an enterokinase cleavage site (DDDDK).

The full sequence of the expressed fusion protein is:

VPIQKVQDDT KTLIKTIVTR INDISHTQSV SSKQKVTGLD
FIPGLHPILT LSKMDQTLAV YQQILTSMPS RNVIQISNDL
ENLRDLLHVL AFSKSCHLPW ASGLETLDSL GGVLEASGYS
TEVVALSRLQ GSLQDMLWQL DLSPGCSKSR SKSRASGSDV
KDDDDKHGEG TFTSDVSSYL EGEAVKEFIA WLVEKVKEFL
G

The construct was transformed into the in-house engineered TKO Prc^{-/-} strain (based on a strain published in the patent application (WO 2010/052335 A1) with an additional knock out of the Prc protease). The transformed clone was grown at 37° C. in shaking flasks with EC1 media to OD₆₀₀˜1. Expression was induced with 0.5 mM IPTG for 4 hr and the cells were harvested. The fusion protein was expressed as inclusion bodies. Cells were resuspended in lysis buffer (1:10, w/w) containing 50 mM Tris, pH 8.0, and lysed by French press. Inclusion bodies were collected by centrifugation at 20,000 g for 1 hr at 4° C., and solubilized to a concentration of 4 mg/ml in buffer containing 50 mM Tris, 8M urea, pH 8.0 and incubated at 4° C. for 3 hrs. After centrifugation at 20,000 g for 30 min, the solubilized Leptin-Compound92 fusion was diluted 8 fold into refolding buffer containing 20 mM Tris, pH 8.0 and enterokinase was then added for cleavage of the leptin tag.

The reaction, which was performed at 25° C. for 16 hrs, resulted in leptin-free Compound 92 as leptin was cleaved from the fusion protein. The enterokinase cleavage occurred concomitantly with the refolding process. The refolding and cleavage process seemed optimal when the ratio between enterokinase and Leptin-Compound92 fusion was 1:2000.

Considering the inhibition of enterokinase by urea, the concentration of urea should not exceed 2M in the refolding buffer. In this case 1M urea was used in the refolding buffer. Finally, Compound 92 was purified by QFF anion exchange chromatography followed by FEF reverse phase chromatography.

In details, a QFF column (CV=5 mL) was selected as the first chromatographic step using a sample buffer containing 50 mM Tris, 1M Urea, pH 8.0. Two buffers were used as mobile phases, Buffer A contained 20 mM Tris, pH 8.0 while buffer B contained 20 mM Tris, 0.5M NaCl, pH 8.0. A flow rate of 5 ml/min was used throughout the chromatographic run with 10 CV 100% A and 10 CV NaCL gradient of 0% B-100% B (a mixture of A and B during the gradient e.g. at the point of 0% B the concentration of A would be 100%, while at the point of 100% B the concentration of A would be 0%) followed by 10CV 100% B. Compound 92 was captured and separated by QFF successfully. Prior to FEF chromatography, 20% ETOH was added into the QFF pool for the application of FEF. Purification of Compound 92 by FEF (CV=3.14 ml) was conducted using a sample buffer containing 50 mM Tris, 20% ETOH, pH 8.0. Two buffers were used as mobile phases. Buffer A contained 20 mM Tris, 20% ETOH, pH 8.0 while buffer B contained 20 mM Tris, 90% ETOH, pH 8.0. A flow rate of 1 mL/min was used throughout the chromatographic run with 10 CV 100% A, 2 CV 42.8% B, 10 CV of gradient from 42.8% B to 85.7% B, 1 CV of gradient from 85.7% B to 100% B and 3CV 100% B.

One milligram of purified Compound 92 was collected following the above chromatographic procedures. The measured molecular weight of Compound 92 by LC/MS was in good agreement with the theoretical value (measured: 3900.9795, theoretical: 3900.95).

### Compound 93

[Aib8,Glu23,Val25,Glu34,Lys35,Phe36,Lys37]-GLP-1-(7-37)-peptidyl-Glu-Phe-Phe amide

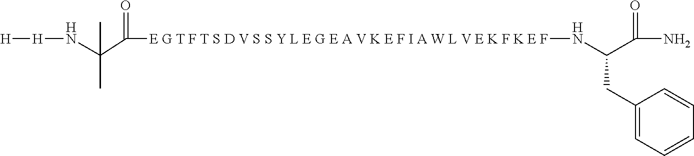

Preparation method: SPPS_P, cleavage CP_M1.

LCMS (method: LCMS_{—}4): Calc. 3956 m/z. Found: m/3: 1319, m/4: 990; m/5 792; Rt-uv=2.6 min.

UPLC (B4_{—}1): Rt=10.0 min.

### Compound 94

[Aib8,Glu23,Val25,Gln26,Glu34,Ala35,Val36,Ala371-GLP-1-(7-37)-peptidyl-Glu-Phe-Leu amide

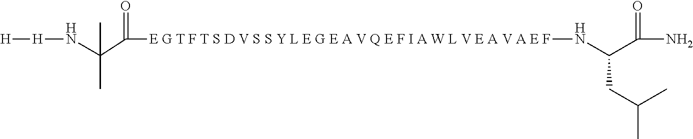

Preparation method: SPPS_P, cleavage CP_M1.

LCMS (method: LCMS_{—}4): Calc. 3759 m/z. Found: m/2: 1880, m/3: 1254, m/4: 941; Rt-uv=12.8 min.

UPLC (B4_{—}1): Rt=3.3 min.

### Compound 95

[Aib8,Glu23,Val25,Gln26,Glu34,Gln35,Aib36,Gln371-GLP-1-(7-37)-peptidyl-Glu-Phe-Leu amide

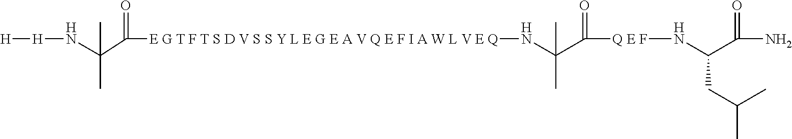

Preparation method: SPPS_P, cleavage CP_M1.

LCMS (method: LCMS_{—}4): Calc. 3859 m/z. Found: m/2: 1930, m/3: 1287; Rt-uv=2.92 min.

UPLC (B4_{—}1): Rt=11.2 min.

### Compound 96

[Aib8,Glu23,Lys24,Val25,Glu30,His31,Glu34,Lys35,Phe36,Lys37]-GLP-1-(7-37)-peptidyl-Glu-Phe-Phe amide

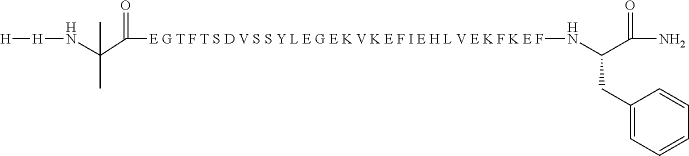

Preparation method: SPPS_P, cleavage CP_M1.

LCMS (method: LCMS_{—}4): Calc. 4022 m/z. Found: m/3: 1341, m/4: 1006; m/5 805, Rt-uv=2.2 min.

UPLC (B4_{—}1): Rt=8.3 min.

### Compound 97

[Aib8,Glu23,Val25,Glu34,Lys35,Aib36,Lys37]-GLP-1-(7-37)-peptidyl-Glu-Phe-Leu-Glu-Lys-Val-Lys-Glu-Phe

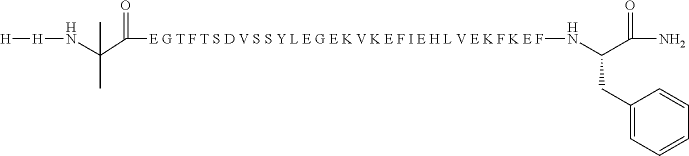

Preparation method: SPPS_P, cleavage CP_M1.

LCMS (method: LCMS_{—}4): Calc. 4621.3 m/z. Found: m/3: 1542, m/4: 1156, m/5. 925.

UPLC (B4_{—}1): Rt=9.6 min.

### Compound 98

[Aib8,Glu23,Val25,Glu34,Lys35,Aib36,Lys37]-GLP-1-(7-37)-peptidyl-Glu-Phe-Leu-Glu-Lys-Val

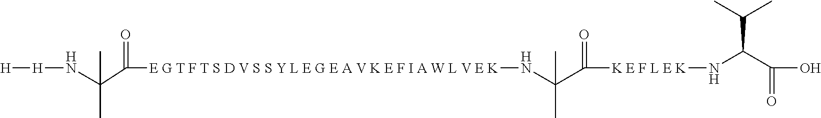

Preparation method: SPPS_P, cleavage CP_M1.

LCMS (method: LCMS_{—}4): Calc. 4216 m/z. Found: m/3: 1406, m/4: 1055, m/5 844.

UPLC (B4_{—}1): Rt=9.8 min.

### Compound 99

[Aib8,Glu23,Val25,Glu34,Lys35,Aib36,Lys37]-GLP-1-(7-37)-peptidyl-Glu-Phe-Leu-Glu-Lys

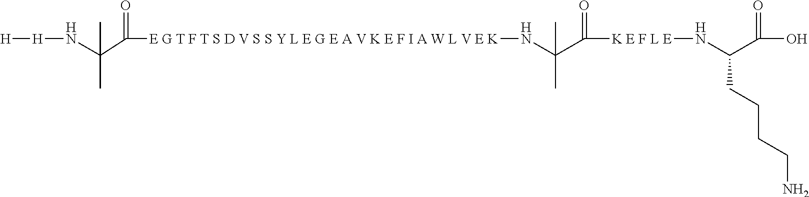

Preparation method: SPPS_P, cleavage CP_M1.

LCMS (method: LCMS_{—}4): Calc. 4118 m/z. Found: m/3: 1374, m/4: 1030, m/5. 825.

UPLC (B4_{—}1): Rt=9.1 min.

### Compound 100

[Aib8,Glu23,Val25,Glu34,Lys35,Aib36,Lys37]-GLP-1-(7-37)-peptidyl-Glu-Phe-Leu-Glu

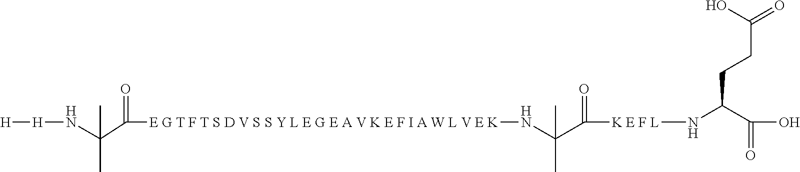

Preparation method: SPPS_P, cleavage CP_M1.

LCMS (method: LCMS_{—}4): Calc. 3989 m/z. Found: m/3: 1331, m/4: 998; m/5 799.

UPLC (B4_{—}1): Rt=9.6 min.

### Compound 101

[Aib8,Pro22,Glu23,Val25,Glu34,Lys35,Aib36,Lys37]-GLP-1-(7-37)-peptidyl-Glu-Phe-Leu amide

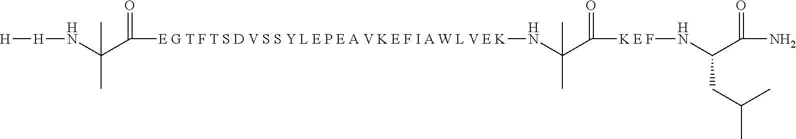

Preparation method: SPPS_P, cleavage CP_M1.

LCMS (method: LCMS_{—}4): Calc. 3899 m/z. Found: m/3. 1301, m/4. 976, m/5. 791.

UPLC (B4_{—}1): Rt=9.8 min.

### Compound 102

[Aib8,Val25,Gln27,Gln34,Lys35,Aib36,Lys37]-GLP-1-(7-37)-peptidyl-Gln-Phe-Leu amide

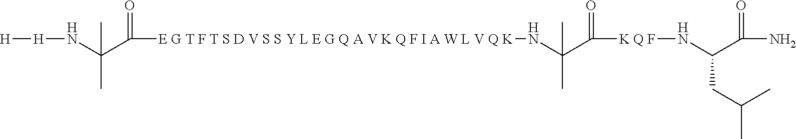

Preparation method: SPPS_P, cleavage CP_M1.

LCMS (method: LCMS_{—}4): Calc. 3855 m/z. Found: m/3: 1286, m/4: 965; m/5. 772.

UPLC (B4_{—}1): Rt=9.6 min.

### Compound 103

[Lys24,Glu34,Val36,Lys37]-GLP-1-(7-37)-peptidyl-Glu-Phe-Leu amide

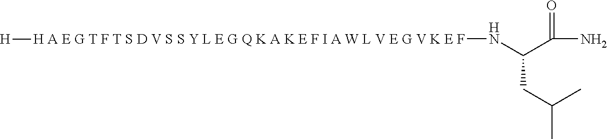

Preparation method: SPPS_P, cleavage CP_M1.

LCMS (method: LCMS_{—}4): Calc. 3816 m/z. Found: m/3: 1273, m/4: 955, m/5. 764.

UPLC (B4_{—}1): Rt=9.01 min.

### Compound 104

[Glu34,Lys35,Val36,Lys37]-GLP-1-(7-37)-peptidyl-Glu-Phe-Leu amide

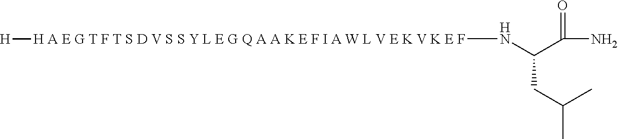

Preparation method: SPPS_P, cleavage CP_M1.

LCMS (method: LCMS_{—}4): Calc. 3830 m/z. Found: m/3: 1273, m/4: 959; m/5 767.

UPLC (B4_{—}1): Rt=9.5 min.

### Compound 105

[Glu34,Lys35,Val36,Lys37]-GLP-1-(7-37)-peptidyl-Glu-Phe-His amide

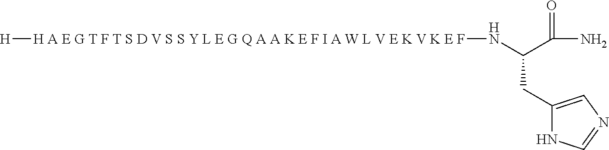

Preparation method: SPPS_P, cleavage CP_M1.

LCMS (method: LCMS_{—}4): Calc. 3854 m/z. Found: m/3: 1286, m/4: 964, m/5. 772.

UPLC (B4_{—}1): Rt=7.6 min.

### Compound 106

[Glu34,Lys35,Val36,Lys37]-GLP-1-(7-37)-peptidyl-Glu-Phe-Leu-Glu amide

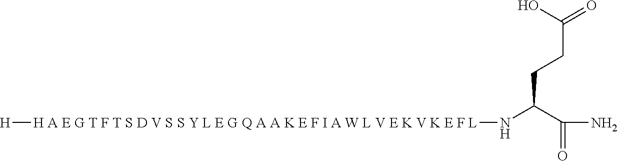

Preparation method: SPPS_P, cleavage CP_M1.

LCMS (method: LCMS_{—}4): Calc. 3960 m/z. Found: m/3: 1321, m/4: 991.

UPLC (B4_{—}1): Rt=9.6 min.

### Compound 107

[Glu34,Lys35,Val36,Lys37]-GLP-1-(7-37)-peptidyl-Glu-His-Leu amide

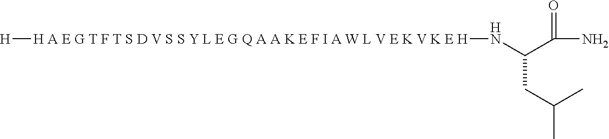

Preparation method: SPPS_P, cleavage CP_M1.

LCMS (method: LCMS_{—}4): Calc. 3820 m/z. Found: m/3: 1275, m/4: 956; m/5. 765.

UPLC (B4_{—}1): Rt=7.6 min.

### Compound 108

[Glu34,Lys35,Val361-GLP-1-(7-37)-peptidyl-Glu-Phe-Leu amide

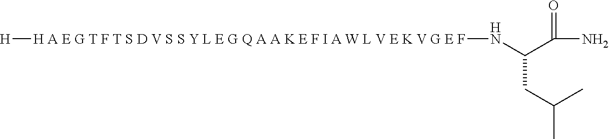

Preparation method: SPPS_P, cleavage CP_M1.

LCMS (method: LCMS_{—}4): Calc. 3759 m/z. Found: m/2: 1881, m/3: 1254, m/4: 941; m/5 758.

UPLC (B4_{—}1): Rt=9.6 min.

### Compound 109

[Aib8,Glu22,Glu23,Val25,Glu34,Lys35,Val36,Lys37]-GLP-1-(7-37)-peptidyl-Glu-Leu-Leu amide

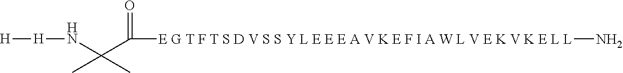

Preparation method: SPPS_P, cleavage CP_M1.

LCMS (method: LCMS_{—}4): Calc. 3912 m/z. Found: m/2: 1957, m/3: 1305, m/4: 979; Rt-uv=2.7 min.

UPLC (B4_{—}1): Rt=10.0 min.

### Compound 110

[Aib8,Glu23,Val25,Glu34,Lys35,Val36,Lys37]-GLP-1-(7-37)-peptidyl-Glu-Lvs amide

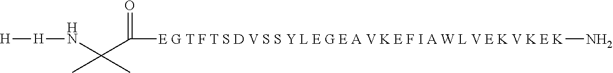

Preparation method: SPPS_P, cleavage CP_M1.

LCMS (method: LCMS_{—}4): Calc. 3741 m/z. Found: m/2: 1872, m/3: 1248, m/4: 937; Rt-uv=2.0 min.

UPLC (B4_{—}1): Rt=7.5 min.

### Compound 111

[Gly8,Glu22,Glu23,Val25,Glu34,Lys35,Val36,Lys37]-GLP-1-(7-37)-peptidyl-Glu-Lys-Leu amide

### ^{H}-HGEGTFTSDVSSYLEEEAVKEFIAWLVEKVKEKL-NH₂

Preparation method: SPPS_P, cleavage CP_M1.

LCMS (method: LCMS_{—}4): Calc. 3898 m/z. Found: m/2: 1971, m/3: 1314, m/4: 986; Rt-uv=2.7 min.

UPLC (B4_{—}1): Rt=10.3 min.

### Compound 112

[Aib8,Glu23,Val25,Glu30,Glu34, Lvs35,Val36, Lvs371-GLP-1-(7-37)-peptidyl-Glu-Lvs-Leu amide

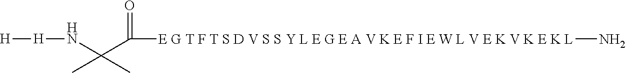

Preparation method: SPPS_P, cleavage CP_M1.

LCMS (method: LCMS_{—}4): Calc. 3912 m/z. Found: m/2: 1958, m/3: 1305, m/4: 979; Rt-uv=2.2 min.

UPLC (B4_{—}1): Rt=8.1 min.

### Compound 113

[Aib8,Glu23,Val25,Glu34,Lys35,Gly36,Lys37]-GLP-1-(7-37)-peptidyl-Glu-Phe-Leu amide

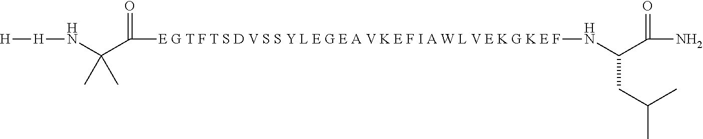

Preparation method: SPPS_P, cleavage CP_M1.

LCMS (method: LCMS_{—}4): Calc. 3831 m/z. Found: m/3, 1278, m/4. 959, m/5. 767.

UPLC (B4_{—}1): Rt=8.6 min.

### Compound 114

[Aib8,Glu23,Val25,Glu34,Lys35,Val36,Lys37]-GLP-1-(7-37)-peptidyl-Asp-Phe-Leu amide

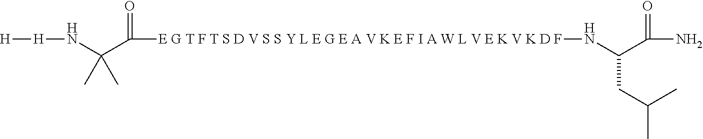

Preparation method: SPPS_P, cleavage CP_M1.

LCMS (method: LCMS_{—}4): Calc. 3859 m/z. Found: m/4: 966; Rt-uv=2.5 min.

UPLC (B4_{—}1): Rt=9.9 min.

### Compound 115

[Aib8,Glu23,Val25,Glu34,Lys35,Val36,Lys37]-GLP-1-(7-37)-peptidyl-Glv-Phe-Leu amide

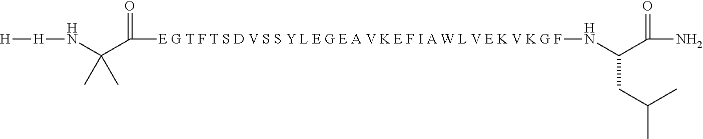

Preparation method: SPPS_P, cleavage CP_M1.

LCMS (method: LCMS_{—}4): Calc. 3801 m/z. Found: m/3: 1268, m/4: 951, m/5: 761.

UPLC (B4_{—}1): Rt=9.4 min.

### Compound 116

[Aib8,Glu23,Val25,Glu34,Lys35,Val36,Lys37]-GLP-1-(7-37)-peptidyl-Glu-Glu-Leu amide

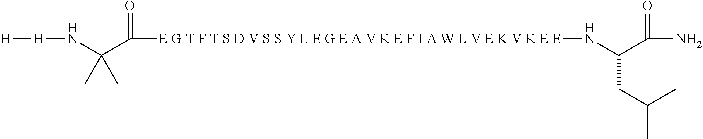

Preparation method: SPPS_P, cleavage CP_M1.

LCMS (method: LCMS_{—}4): Calc. 3855 m/z. Found: m/3: 1286, m/4: 965; Rt-uv=2.3 min.

UPLC (B4_{—}1): Rt=8.7 min.

### Compound 117

[Aib8,Glu23,Val25,Glu34,Lys35,Val36,Lys37]-GLP-1-(7-37)-peptidyl-Glu-Gly-Leu amide

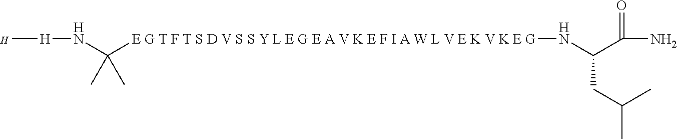

Preparation method: SPPS_P, cleavage CP_M1

LCMS (method: LCMS_{—}4): Calc. 3783 m/z. Found: m/3 1262, m/4: 947, m/5. 758.

UPLC (B4_{—}1): Rt=8.5 min.

### Compound 118

[Aib8,Glu23,Val25,Glu34,Lys35,Val36,Lys37]-GLP-1-(7-37)-peptidyl-Glu-Lys-Leu amide

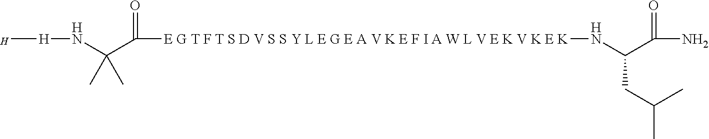

Preparation method: SPPS_P, cleavage CP_M1.

LCMS (method: LCMS_{—}4): Calc. 3854 m/z. Found: m/3: 1286, m/4: 964; Rt-uv=2.1 min.

UPLC (B4_{—}1): Rt=8.2 min.

### Compound 119

[Aib8,Glu23,Val25,Glu34,Lys35,Val36,Lys37]-GLP-1-(7-37)-peptidyl-Glu-Leu-Leu amide

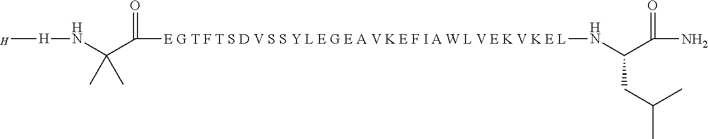

Preparation method: SPPS_P, cleavage CP_M1.

LCMS (method: LCMS_{—}4): Calc. 3839 m/z. Found: m/3: 1281, m/4: 961, m/5 769; Rt-uv=2.6 min.

UPLC (B4_{—}1): Rt=10.4 min.

### Compound 120

[Aib8,Glu23,Val25,Glu34,Lys35,Val36,Lys37]-GLP-1-(7-37)-peptidyl-Glu-Val-Leu amide

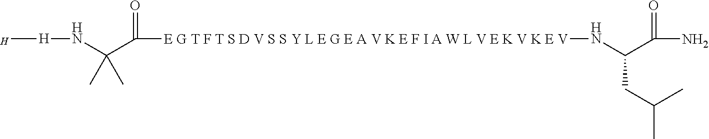

Preparation method: SPPS_P, cleavage CP_M1.

LCMS (method: LCMS_{—}4): Calc. 3825 m/z. Found: m/3: 1276, m/4: 957, m/5 766; Rt-uv=2.6 min.

UPLC (B4_{—}1): Rt=10.1 min.

### Compound 121

[Aib8,Glu23,Val25,Glu34,Lys35,Val36,Lys37]-GLP-1-(7-37)-peptidyl-Glu-Phe-Phe amide

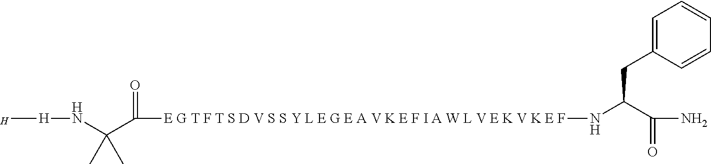

Preparation method: SPPS_P, cleavage CP_M1.

LCMS (method: LCMS_{—}4): Calc. 3907 m/z. Found: m/3: 1303, m/4: 977, m/5 782; Rt-uv=2.6 min.

UPLC (B4_{—}1): Rt=16.8 min.

### Compound 122

[Glu34,Lys35,Val36,Lys37]-GLP-1-(7-37)-peptidyl-Glu-Phe amide

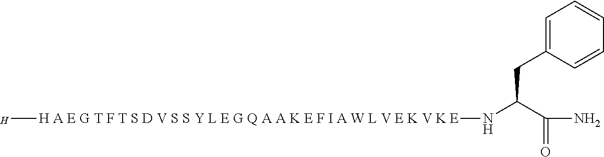

Preparation method: SPPS_P, cleavage CP_M1.

LCMS (method: LCMS_{—}4): Calc. 3717 m/z. Found: m/3. 1240, m/4: 930, m/5. 745:

UPLC (B4_{—}1): Rt=8.2 min.

### Compound 123

[Glu23,Val25,Glu34,Lys35,Val36,Lys37]-GLP-1-(7-37)-peptidyl-Glu-Phe-Leu-Glu-Lys

### ^{H}-HAEGTFTSDVSSYLEGEAVKEFIAWLVEKVKEFLEK-^{CH}

Preparation method: SPPS_P, cleavage CP_M1.

LCMS (method: LCMS_{—}4): Calc. 4118 m/z. Found: m/3: 1373, m/4:1030, m/5. 825.

UPLC (B4_{—}1): Rt=9.4 min.

### Compound 124

[Glu34,Lys35,Val36,Lys37]-GLP-1-(7-37)-peptidyl-Glu-Phe-Leu-Glu-Lys

### ^{H}-HAEGTFTSDVSSYLEGQAAKEFIAWLVEKVKEFLEK-^{CH}

Preparation method: SPPS_P, cleavage CP_M1.

LCMS (method: LCMS_{—}4): Calc. 4089 m/z. Found: m/3: 1364, m/4: 1023; m/5. 819.

UPLC (B4_{—}1): Rt=8.9 min.

### Compound 125

[Glu23,Val25,Glu34,Lys35,Val36,Lys37]-GLP-1-(7-37)-peptidyl-Glu-His-Leu-Glu-Lys

### ^{H}-HAEGTFTSDVSSYLEGEAVKEFIAWLVEKVKEHLEK-^{CH}

Preparation method: SPPS_P, cleavage CP_M1.

LCMS (method: LCMS_{—}4): Calc. 4108 m/z. Found: m/3: 1370, m/4: 1028; Rt-uv=2.93 min.

UPLC (B4_{—}1): Rt=7.7 min.

### Compound 126

[Glu34,Lys35,Val36,Lys37]-GLP-1-(7-37)-peptidyl-Glu-His-Leu-Glu-Lys

### ^{H}-HAEGTFTSDVSSYLEGQAAKEFIAWLVEKVKEHLEK-^{CH}

Preparation method: SPPS_P, cleavage CP_M1.

LCMS (method: LCMS_{—}4): Calc. 4078 m/z. Found: m/3: 1360, m/4: 1021; m/5. 816.

UPLC (B4_{—}1): Rt=7.31 min.

### Compound 127

[Glu34,Val36,Lys37]-GLP-1-(7-37)-peptidyl-Glu-Phe-Leu-Glu-Lys

### ^{H}-HAEGTFTSDVSSYLEGQAAKEFIAWLVEGVKEFLEK-^{CH}

Preparation method: SPPS_P, cleavage CP_M1.

LCMS (method: LCMS_{—}4): Calc. 4018 m/z. Found: m/3: 1340, m/4: 1005; m/5. 805.

UPLC (B4_{—}1): Rt=9.4 min.

### Compound 128

[Glu34,Lys35,Val361-GLP-1-(7-37)-peptidyl-Glu-Phe-Leu-Glu-Lys ^{H}-HAEGTFTSDVSSYLEGQAAKEFIAWLVEKVGEFLEK-^{CH}

Preparation method: SPPS_P, cleavage CP_M1.

LCMS (method: LCMS_{—}4): Calc. 4018 m/z. Found: m/3: 1340, m/4: 1006; m/5. 805.

UPLC (B4_{—}1): Rt=9.1 min.

### Compound 129

[Glu34,Lys35,Val36,Lys37]-GLP-1-(7-37)-peptidyl-Glu-Phe-Leu-Asp-Lys

### ^{H}-HAEGTFTSDVSSYLEGQAAKEFIAWLVEKVKEFLDK-^{CH}

Preparation method: SPPS_P, cleavage CP_M1.

LCMS (method: LCMS_{—}4): Calc. 4075 m/z. Found: m/3: 1359, m/4: 1019; m/5. 816.

UPLC (B4_{—}1): Rt=8.8 min.

### Compound 130

[Glu34,Lys35,Val36,Lys37]-GLP-1-(7-37)-peptidyl-Glu-Phe-Leu-Glu-Arg

### ^{H}-HAEGTFTSDVSSYLEGQAAKEFIAWLVEKVKEFLER-^{CH}

Preparation method: SPPS_P, cleavage CP_M1.

LCMS (method: LCMS_{—}4): Calc. 4117 m/z. Found: m/3: 1373, m/4: 1030, m/5. 824.

UPLC (B4_{—}1): Rt=9.0 min.

### Compound 131

[Glu34,Lys35,Val36,Lys37]-GLP-1-(7-37)-peptidyl-Glu-Phe-Tyr amide

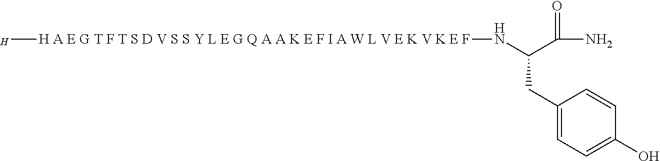

Preparation method: SPPS_P, cleavage CP_M1.

LCMS (method: LCMS_{—}4): Calc. 3880 m/z. Found: m/3: 1294, m/4: 971; m/5. 777.

UPLC (B4_{—}1): Rt=8.3 min.

### Compound 132

[Glu22,Glu23,Val25,Glu34,Lys35,Val36,Lys37]-GLP-1-(7-37)-peptidyl-Glu-Phe amide

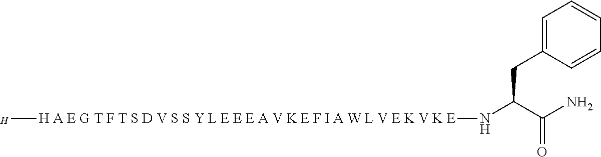

Preparation method: SPPS_P, cleavage CP_M1.

LCMS (method: LCMS_{—}4): Calc. 3818 m/z. Found: m/3: 1274, m/4: 955; m/5. 765.

UPLC (B4_{—}1): Rt=8.6 min.

### Compound 133

[Gly8,Glu22,Glu23,Val25,Glu34,Lys35,Val36,Lys37]-GLP-1-(7-37)-peptidyl-Glu-Phe amide

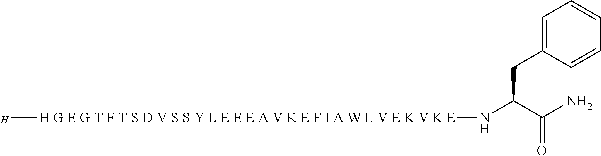

Preparation method: SPPS_P, cleavage CP_M1.

LCMS (method: LCMS_{—}4): Calc. 3804 m/z. Found: m/3: 1269, m/4: 952; m/5 762 Rt-uv=2.2 min.

UPLC (B4_{—}1): Rt=8.8 min.

### Compound 134

[Aib8,Glu22,Glu23,Val25,Glu34,Lys35,Val36,Lys37]-GLP-1-(7-37)-peptidyl-Glu-Phe amide

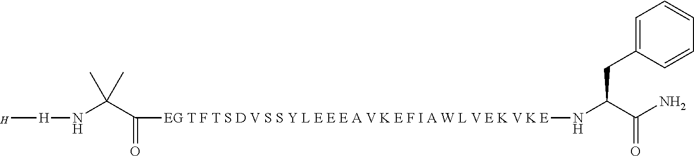

Preparation method: SPPS_P, cleavage CP_M1.

LCMS (method: LCMS_{—}4): Calc. 3832 m/z. Found: m/3: 1278, m/4: 959; Rt-uv=2.2 min.

UPLC (B4_{—}1): Rt=8.8 min.

### Compound 135

[Glu22,Glu23,Val25,Glu34,Lys35,Val36,Lys37]-GLP-1-(7-37)-peptidyl-Glu-Phe-Leu amide

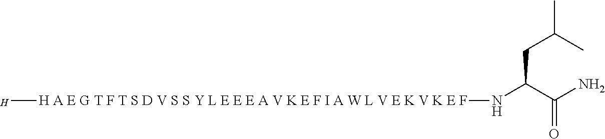

Preparation method: SPPS_P, cleavage CP_M1.

LCMS (method: LCMS_{—}4): Calc. 3931 m/z. Found: m/3: 1311, m/4: 983, m/5 786; Rt-uv=2.4 min.

UPLC (B4_{—}1): Rt=10.0 min.

### Compound 136

[Gly8,Glu22,Glu23,Val25,Glu34,Lys35,Val36,Lys37]-GLP-1-(7-37)-peptidyl-Glu-Phe-Leu amide

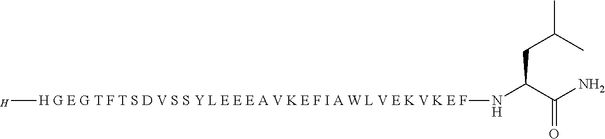

Preparation method: SPPS_P, cleavage CP_M1.

LCMS (method: LCMS_{—}4): Calc. 3917 m/z. Found: m/3: 1307, m/4: 980; Rt-uv=2.4 min.

UPLC (B4_{—}1): Rt=10.1 min.

### Compound 137

[Aib8,Glu22,Glu23,Val25,Glu34,Lys35,Val36,Lys37]-GLP-1-(7-37)-peptidyl-Glu-Phe-Leu amide

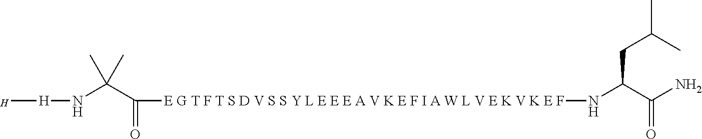

Preparation method: SPPS_P, cleavage CP_M1.

LCMS (method: LCMS_{—}4): Calc. 3946 m/z. Found: m/3: 1316, m/4: 987; m/5. 790.

UPLC (B4_{—}1): Rt=9.9 min.

### Compound 138

[Glu23,Val25,Arg26,Glu34,Arg35,Val36,Arg371-GLP-1-(7-37)-peptidyl-Glu-Phe-Leu-Glu-Lys

### ^{H}-HAEGTFTSDVSSYLEGEAVREFIAWLVERVREFLEK-^{CH}

Preparation method: SPPS_P, cleavage CP_M1.

LCMS (method: LCMS_{—}4): Calc. 4202 m/z. Found: m/3: 1401, m/4: 1052; m/5. 841.

UPLC (B4_{—}1): Rt=9.1 min.

### Compound 139

[Glu23,Val25,Arg26,His34,Arg35,Val36,Arg371-GLP-1-(7-37)-peptidyl-Glu-Phe-Leu-Glu-Lys

### ^{H}-HAEGTFTSDVSSYLEGEAVREFIAWLVHRVREFLEK-^{CH}

Preparation method: SPPS_P, cleavage CP_M1.

LCMS (method: LCMS_{—}4): Calc. 4210 m/z. Found: m/3: 1404, m/4: 1053; m/5. 843.

UPLC (B4_{—}1): Rt=8.6 min.

### Compound 140

[Glu34,His35,Val36,Lys37]-GLP-1-(7-37)-peptidyl-Glu-Phe-Leu amide

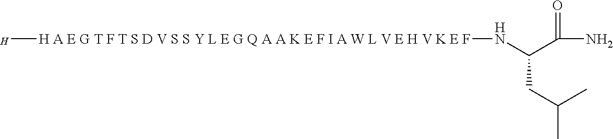

Preparation method: SPPS_P, cleavage CP_M1.

LCMS (method: LCMS_{—}4): Calc. 3839 m/z. Found: m/3: 1281, m/4: 961; m/5. 769.

UPLC (B4_{—}1): Rt=9.1 min.

### Compound 141

[Glu34,Lys35,Val36,His371-GLP-1-(7-37)-peptidyl-Glu-Phe-Leu amide

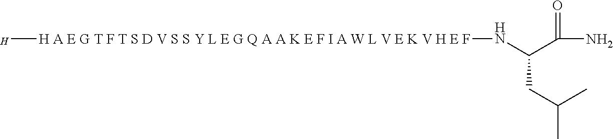

Preparation method: SPPS_P, cleavage CP_M1.

LCMS (method: LCMS_{—}4): Calc. 3839 m/z. Found: m/3: 1281, m/4: 961; m/5. 769.

UPLC (B4_{—}1): Rt=9.1 min.

### Example 2

### Functional GLP-1 Receptor Assay

### In Vitro Potency (CRE Luciferase; Whole Cells)

The purpose of this example is to test the activity, or potency, of the GLP-1 receptor agonist peptides in vitro. The in vitro potency is the measure of human GLP-1 receptor activation in a whole cell assay.

The potencies of the GLP-1 receptor agonist peptides representative of the invention, i.e. Compounds 1-61, were determined as described below. GLP-1(7-37) was included for comparison.

### Principle

In vitro potency was determined by measuring the response of the human GLP-1 receptor in a reporter gene assay. The assay was performed in a stably transfected BHK cell line that expresses the human GLP-1 receptor and contains the DNA for the cAMP response element (CRE) coupled to a promoter and the gene for firefly luciferase (CRE luciferase). When the human GLP-1 receptor is activated it results in the production of cAMP, which in turn results in the luciferase protein being expressed. When assay incubation is completed the luciferase substrate (luciferin) is added and the enzyme converts luciferin to oxyluciferin to produce bioluminescence. The luminescence is measured as the readout for the assay.

In order to test the binding of the peptides to albumin, the assay was performed in the absence of serum albumin as well as in the presence of a considerably higher concentration of serum albumin (1.0% final assay concentration). An increase of the in vitro potency, EC₅₀ value, in the presence of serum albumin would indicate an affinity to serum albumin and represents a method to predict a protracted pharmacokinetic profile of the test substance in animal models.

### Cell Culture and Preparation

The cells used in this assay (clone FCW467-12A/KZ10-1) were BHK cells with BHKTS13 as a parent cell line. The cells were derived from a clone (FCW467-12A) that expresses the human GLP-1 receptor and were established by further transfection with CRE luciferase to obtain the current clone.

The cells were cultured at 5% CO₂ in Cell Culture Medium. They were aliquoted and stored in liquid nitrogen. Before each assay an aliquot is taken up and washed twice in PBS before being suspended at the desired concentration in the assay specific buffer. For 96-well plates the suspension was made to give a final concentration of 5×10³ cells/well.

### Materials

The following chemicals were used in the assay: Pluronic F-68 (10%) (Gibco 2404), human serum albumin (HSA) (Sigma A9511), ovalbumin (Sigma A5503), DMEM w/o phenol red (Gibco 11880-028), 1 M Hepes (Gibco 15630), Glutamax 100× (Gibco 35050) and steadylite plus (Perkin Elmer 6016757).

Cell Culture Medium consisted of DMEM medium with 10% FBS (Fetal Bovine Serum), 1 mg/ml G418, 240 nM MTX (methotrexate) and 1% pen/strep (penicillin/streptomycin). Assay Medium consisted of DMEM w/o phenol red, 10 mM Hepes and 1× Glutamax. The 1% Assay Buffer consisted of 2% ovalbumin, 0.2% Pluronic F-68 and 2% HSA in assay medium. The 0% Assay Buffer consisted of 2% ovalbumin and 0.2% Pluronic F-68 in Assay Medium.

### Procedure

1) Cell stocks were thawed in a 37° C. water bath.

2) Cells were washed three times in PBS.

3) The cells were counted and adjusted to 5×10³ cells/50 μl (1×10⁵ cells/ml) in Assay Medium. A 50 μl aliquot of cells was transferred to each well in the assay plate.

4) Stocks of the test compounds and reference compounds were diluted to a concentration of 0.2 μM in 0% Assay Buffer for the 0% HSA CRE luciferase assay and 1% Assay Buffer for the 1% HSA CRE luciferase assay. Compounds were diluted 10-fold to give the following concentrations: 2×10⁻⁷ M, 2×10⁻⁸ M; 2×10⁻⁹ M, 2×10⁻¹⁹ M, 2×10⁻¹¹ M, 2×10⁻¹² M, 2×10⁻¹³ M, and 2×10⁻¹⁴ M.

5) A 50 μl aliquot of compound or blank was transferred from the dilution plate to the assay plate. Compounds were tested at the following final concentrations: 1×10⁻⁷ M, 1×10⁻⁸ M; 1×10⁻⁹ M, 1×10⁻¹° M, 1×10⁻¹¹ M, 1×10⁻¹² M, 1×10⁻¹³ M, and 1×10⁻¹⁴ M.

6) The assay plate was incubated for 3 h in a 5% CO₂ incubator at 37° C.

7) The assay plate was removed from the incubator and allowed to stand at room temperature for 15 min.

8) A 100 μl aliquot of steadylite plus reagent was added to each well of the assay plate (reagent was light sensitive).

9) Each assay plate was covered with aluminum foil to protect it from light and shaken for 30 min at room temperature.

10) Each assay plate was read in a Packard TopCount NXT instrument.

### Calculations and Results

The data from the TopCount instrument were transferred to GraphPad Prism software. The software performs a non-linear regression (log(agonist) vs response). EC₅₀ values which were calculated by the software and reported in pM are shown in Table 6, below.

A minimum of two replicates was measured for each sample. The reported values are averages of the replicates.

### Example 3

### Physical Stability Assay

### Dynamic Light Scattering

Physical stability can be assessed by dynamic and static light scattering. In dynamic light scattering, microsecond fluctuations in scattered laser light incident on a aqueous sample is detected and transformed into diffusion coefficients (Df) of the individual species via the so-called autocorrelation function. For convenience, the diffusion coefficients are typically reported in hydrodynamic radii (Rh) assuming the sample to consist of spherical species. Furthermore, from the radii, an empirical estimate of the molecular weight is obtained. Dynamic light scattering is extremely sensitive and can resolve very small amounts of aggregated species that are undesirable in pharmaceutical formulations. The average, static intensity recorded by the detector also serves as an overall measure of the physical stability of the sample as development of larger species increase the scattered intensity drastically. In order to measure the physical stability of peptides, DLS was applied to aqueous solutions of peptides. Measurements were performed on a Wyatt (Santa Barbara, Calif.) DynaPro DLS plate reader at 25° C., and samples were kept at 37° C. between measurements. Samples were measured for up to two weeks. Measurements were performed in 25-L triplicate in Corning 3540 384-well microtiter plates (Corning, N.Y.) sealed with transparent plastic foil (Thermo Fischer Scientific, Waltham, Mass.) with twenty 10-second acquisitions per measurement. Autocorrelation curves were fitted with a regularization fit in Dynamics 7.1.7.16 and the resulting diffusion coefficients were transformed into hydrodynamic radii and molecular mass assuming a spherical shape (Table 2). Normalized intensities are reported as is.

DLS results for Compound 1 in 20 mM phosphate pH 7.5 buffer at concentrations of 0.9 mg/mL or 45.5 mg/mL (11.5 and 0.23 mM, respectively) shows that Compound 1 forms an oligomer of (molecular weight consistent with approximately 4 monomers), which remained stable throughout the sample period (Tables 1 and 2; FIGS. 10 and 11). The stability and oligomer size did not seem to depend on the sample concentration (in the observed range from 0.9 to 45.4 mg/mL). Judging from the autocorrelation functions and the normalized static intensity, only minute amounts of aggregate were present at any time independent of sample concentration.

TABLE 2
Hydrodynamic radii (average and standard deviation) and normalized
intensity from a sample of Compound 1 at 0.9 mg/mL

| | Rₕ avg. | Rₕ S.D. | Iₙₒᵣₘ avg. | Iₙₒᵣₘ S.D. |
|---|---|---|---|---|
| t (days) | (nm) | (nm) | (kcts) | (kcts) |
| 0 | 2.32 | 0.09 | 1.15E+06 | 5.00E+004 |
| 1 | 2.31 | 0.21 | 1.48E+06 | 1.84E+005 |
| 4 | 1.94 | 0.20 | 3.76E+06 | 2.46E+006 |
| 8 | 2.26 | 0.08 | 1.27E+06 | 1.81E+005 |
| 13 | 2.44 | 0.09 | 2.25E+06 | 4.27E+005 |

TABLE 3
Hydrodynamic radii (average and standard deviation) and normalized
intensity from a sample of Compound 1 at 45.5 mg/mL

| | Rₕ avg. | Rₕ S.D. | Iₙₒᵣₘ avg. | Iₙₒᵣₘ S.D. |
|---|---|---|---|---|
| t (days) | (nm) | (nm) | (kcts) | (kcts) |
| 0 | 2.13 | 0.05 | 8.33E+06 | 5.77E+005 |
| 1 | 2.27 | 0.01 | 9.29E+06 | 5.54E+005 |
| 4 | 2.27 | 0.12 | 1.04E+07 | 7.04E+005 |
| 6 | 2.35 | 0.03 | 6.55E+06 | 1.28E+006 |
| 14 | 2.28 | 0.16 | 7.18E+06 | 1.91E+006 |

### Example 4

### Solubility Assay

pH solubility curves are representations of sample solubility at given pH values in a specific pH range (e.g. 12 points from pH 4 to 9). One approach is to make a pH gradient from a buffer mix consisting of two buffers (e.g. citrate 100 mM/phosphate 200 mM), spike with a concentrated sample stock, and then leave the samples for 2 days at ambient temperature. Then the samples are centrifuged to isolate precipitated sample, and the concentration is measured in the supernatant using, e.g., rpUPLC, UV absorbance, or nitrogen detection. pH solubility curves were made by adding peptide stock (ca. 1.25 in MQ) to a buffer mix (citrate 100 mM/phosphate 200 mM), which makes a pH gradient from 4 to 9 in 12 steps at a sample concentration of 2 mg/mL. Samples were incubated for two days at 25° C., centrifuged at 4700 g, and the concentration measured in the supernatant on a Nanodrop UV spectrophotometer (Thermo Fischer Scientific, Waltham, Mass.) using a theoretical extinction coefficient of 6,990 cm⁻¹M⁻¹.

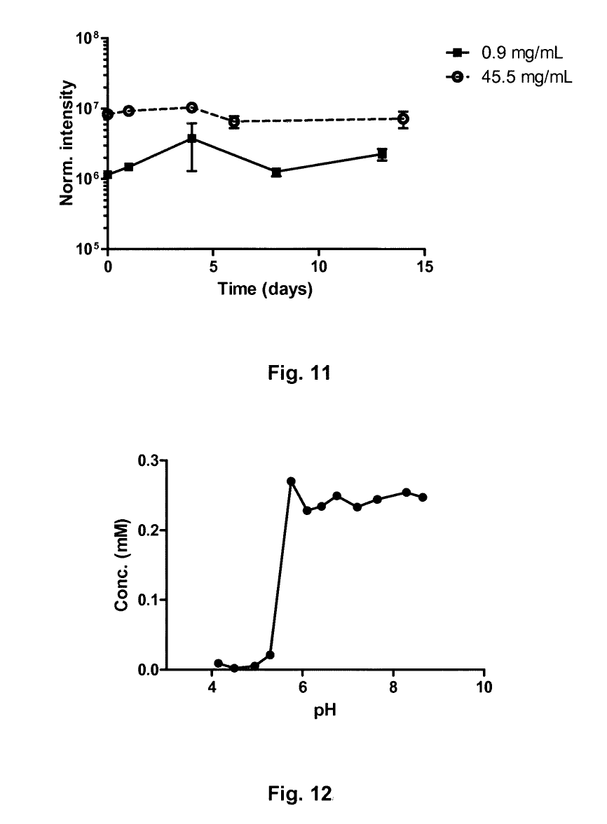

The pH solubility in Table 4 and FIG. 12 display the solubility profile for Compound 1.30 μL sample stock (1.25 mM) was added to the 12 buffer-mix compositions below and pH and concentration measured.

TABLE 4
pH solubility data

| | Citrate 100 mM | Phosphate 200 mM | Conc. |
|---|---|---|---|
| pH meas. | (μL) | (μL) | supernatant |
| 4.15 | 77.4 | 42.6 | 0.009 |
| 4.50 | 70.32 | 49.68 | 0.002 |
| 4.95 | 63.9 | 56.1 | 0.005 |
| 5.29 | 58.2 | 61.8 | 0.021 |
| 5.75 | 53.1 | 66.9 | 0.270 |
| 6.10 | 47.46 | 72.54 | 0.228 |
| 6.42 | 40.68 | 79.32 | 0.234 |
| 6.76 | 32.7 | 87.3 | 0.249 |
| 7.21 | 21.18 | 98.82 | 0.233 |
| 7.65 | 10.98 | 109.02 | 0.244 |
| 8.29 | 5.1 | 114.9 | 0.254 |
| 8.65 | 0 | 120 | 0.247 |

### Example 5

### Mechanical Stress Experiment

### Thioflavin T Fibrillation Assay

In order to determine the physical stability of peptides in solution an accelerated mechanical stress assay was performed. Samples were prepared just before starting the assay. Samples may be prepared in the pH range 3-9 and with peptide concentrations in the range 0-20 mM. Samples may contain buffers such as phosphate, acetate, Tris. Samples may contain antimicrobial preservatives such as phenol or m-cresol.

The pH of the sample was adjusted to the desired value using NaOH and HCl. Thioflavin T was added to the samples from a stock solution in H₂O to a final concentration of 5 μM.

Aliquots of each sample were transferred to a 96 well microtiter plate (Packard OptiPlate™-96, white polystyrene) with 200 μL in each well. For each sample data may be recorded for one well or for two or more replicas (two or more wells). The plate was sealed with Tape Pad (Qiagen).

Incubation at a selected temperature, mechanical stress and measurement of Thioflavin T fluorescence was performed using a Fluoroskan Ascent FL fluorescence platereader (Thermo Labsystems). For mechanical stress orbital shaking at e.g. 960 rpm and amplitude of 1 mm may be used. Fluorescence measurement was performed using excitation through a 444 nm filter and measurement of emission through a 485 nm filter. The fluorescence was measured every 20 minutes during the experiment.

The fluorescence from Thioflavin T was plotted against duration of mechanical stress. A substantial increase in fluorescence from Thioflavin T indicates amyloid fibril formation.

The peptide concentration for each sample was measured before and after the mechanical stress assay. After mechanical stress the samples from the wells were pooled for each sample composition and centrifuged (20000 G, 30 min, 21° C.). The supernatant was removed and optionally filtered (0.22 μm filter) and the peptide concentration was measured. The peptide concentration may be measured using e.g. nitrogen detection, NMR, UV absorbance, rpHPLC or UPLC.

The peptide concentration after mechanical stress divided by the peptide concentration before mechanical stress was calculated and defined as peptide recovery.

The result of mechanical stress experiments for a period of 45 hours at 37° C. for Compound 1 in the following sample compositions:

A. 250 μM peptide, 20 mM phosphate buffer pH 7.5;

B. 250 μM peptide, 20 mM phosphate buffer pH 7.5, 25 mM m-cresol;

C. 250 μM peptide, 20 mM phosphate buffer pH 7.5, 150 mM NaCl;

D. 250 μM peptide, 20 mM phosphate buffer pH 7.5, 25 mM m-cresol, 150 mM NaCl;

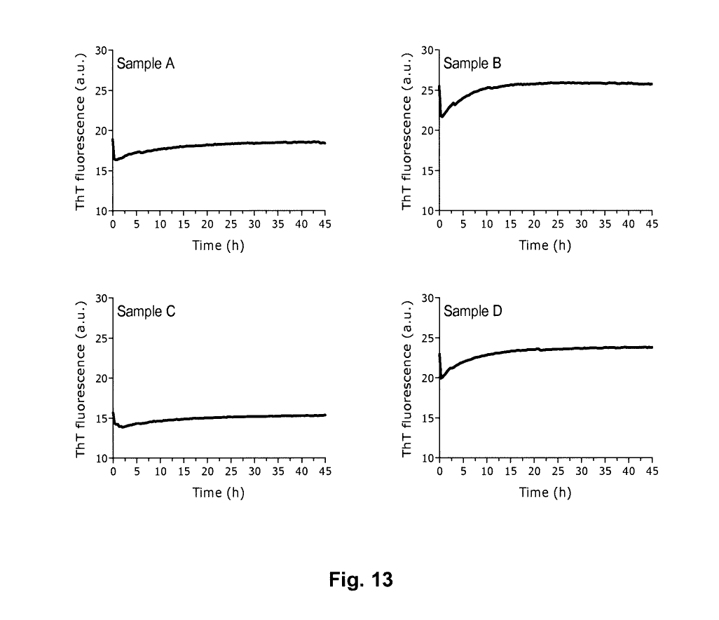

are provided in Table 5 and in FIG. 13.

TABLE 5
Peptide recovery data
Determined after 45 hours of mechanical stress at 37° C. for Sample A:
250 μM peptide, 20 mM phosphate buffer pH 7.5; Sample B: 250 μM
peptide, 20 mM phosphate buffer pH 7.5, 25 mM m-cresol; Sample C:
250 μM peptide, 20 mM phosphate buffer pH 7.5, 150 mM NaCl;
and Sample D: 250 μM peptide, 20 mM phosphate buffer pH 7.5,
25 mM m-cresol, 150 mM NaCl.

| Sample | Peptide recovery (%) |
|---|---|
| A | 103 |
| B | 100 |
| C | 103 |
| D | 103 |

The peptide was physically stable in all sample compositions (A-D), see FIG. 13 in the accelerated mechanical stress assay as no substantial increase of fluorescence from Thioflavin T was observed and full recovery of peptide after 45 hours of stress was found.

This enables a liquid formulation with good physical stability of the peptide.

### Example 6

### Cholesterol Efflux Assay

Cholesterol efflux was assessed in vitro by measuring the capacity of compounds to efflux cholesterol from macrophage cell line, primarily transported via the ABCA1 transporter. 8-(4-Chlorophenyl-thio) adenosine 3′,5′-cyclic monophosphate sodium salt (CPT-cAMP) was used to up-regulate the ABCA1 transporter.

Mouse monocyte/macrophage cell line, RAW 264.7 (ATCC, Cat. # TIB-71), was seeded (40.000 c/well) in 96 well plates (NUNC, cat. #167008) and grown in culture mediad with 3.5 μCi 3H-Cholesterol Cholesterol, [1,2-3H(N)] from PerkinElmer, Cat. #NET139001MC), in DMEM media for 30 hours (37° C., 5% CO₂). Media was removed, cells washed once with assay media made of DMEM (Gibco, Cat. #31966-021) with 1% Penicillin/Streptomycin (GIBCO, Cat. #15140).

Cells were subsequently incubated with assay media containing 0.1% human serum albumin ((Sigma, Cat. # A1887)±0.3 mM CPT-cAMP (Sigma, Cat # C3912) for 18 h (37° C., 5% CO₂). Again media was removed and cells were washed with assay media 2 times. Compounds were diluted in assay media with 0.1% human serum albumin and 100 μl of the final dilution was added to cells in each well and incubated for 4 hrs (37° C., 5% CO₂). At the end of the incubation time, cell-free media was collected, transferred to Optiplate-96 plates. 180 μl scintillation fluid, Microscint 40 (Sigma T9284), was added to Optiplate-96 plates (PerkinElmer, Cat. #6005290), mixed for one minute, allowed to stand for 30 min, and the radioactivity of the labelled cholesterol effluxed into the media was counted in a Topcounter. Radioacivity from the labeled cholesterol in the cells was assessed by lysing the cells in the plate with 1% Triton X-100 (Sigma T9284) for 30 min. The lysate was transferred to Optiplate-96 well plate, 180 μl Microscint 40 was added, mixed for one minute and then allowed to stand for 30 min. The radioactivity of the labelled cholesterol remaining in the cells was counted in a microplate scintillation counter (Topcounter NTX, Perkin Elmer).

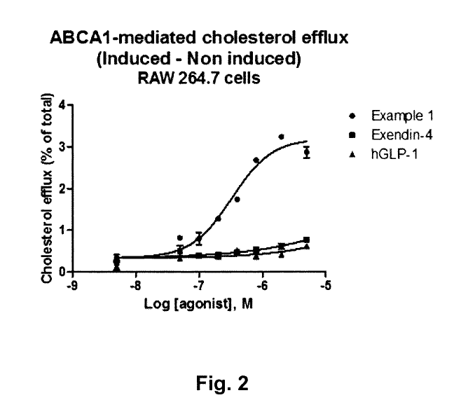

The data from the TopCounter instrument were transferred to GraphPad Prism software. Cholesterol efflux was calculated: cpm media/(cpm media+cpm cell lysate)×100%. ABCA1-mediated efflux was obtained from the difference between induced efflux and non-induced efflux. EC₅₀ values which were calculated by the software and reported in μM are shown in Table 6. FIG. 2 show the cholesterol efflux curves for Compound 1, hGLP-1 and Exendin-4.

A minimum of two replicates was measured for each sample. The reported values are averages of the replicates.

TABLE 6
Cholesterol efflux and GLP-1 receptor potency
of the Compounds of the invention

| | 4 h | 4 h | GLP1R CRE | GLP1R CRE |
|---|---|---|---|---|
| | Cholesterol | Cholesterol | luc 0% HSA | luc 1% HSA |
| Compound | Efflux | Efflux | EC50 (pM) | EC50 (pM) |
| (No.) | EC50 (μM) | Emax (%) | Mean Value | Mean Value |
| Glucagon | | | 7073 | 5440 |
| Exendin-4 | >5 | <10 | 5 | 4 |
| GLP-1(7-37) | >5 | <10 | 13 | 6 |
| L-4F | 2.80 | 108 | | |
| 1 | 0.30 | 100 | 267 | 111 |
| 2 | 0.46 | 83 | 506 | 260 |
| 3 | 0.56 | 82 | 396 | 193 |
| 4 | 0.35 | 100 | 36 | 19 |
| 5 | 0.43 | 111 | 89 | 116 |
| 6 | 0.49 | 92 | 474 | 381 |
| 7 | 0.40 | 90 | 161 | 74 |
| 8 | 0.58 | 100 | 163 | 143 |
| 9 | 0.41 | 87 | 921 | 276 |
| 10 | 1.73 | 79 | 10 | 4 |
| 11 | 2.19 | 135 | 15 | 4 |
| 12 | 0.51 | 86 | 1513 | 552 |
| 13 | 0.40 | 91 | 202 | 104 |
| 14 | 0.30 | 96 | 222 | 62 |
| 15 | 0.57 | 91 | 338 | 101 |
| 16 | 0.68 | 105 | 703 | 162 |
| 17 | 0.98 | 102 | 66 | 20 |
| 18 | 0.54 | 89 | 4876 | 776 |
| 19 | 0.66 | 83 | 47 | 23 |
| 20 | 0.56 | 102 | 155 | 82 |
| 21 | 0.42 | 70 | 763 | 296 |
| 22 | 0.77 | 106 | 17 | 6 |
| 23 | 0.73 | 99 | 71 | 46 |
| 24 | 0.26 | 82 | 1629 | 908 |
| 25 | 0.40 | 68 | 591 | 297 |
| 26 | 0.20 | 87 | 393 | 153 |
| 27 | 0.89 | 93 | 79 | 28 |
| 28 | 0.23 | 78 | 269 | 104 |
| 29 | 0.31 | 81 | 289 | 137 |
| 30 | 0.84 | 75 | 34 | 13 |
| 31 | 0.45 | 84 | 205 | 62 |
| 32 | 0.56 | 85 | 39 | 13 |
| 33 | 0.68 | 87 | 46 | 22 |
| 34 | 1.19 | 94 | 67 | 32 |
| 35 | 1.87 | 48 | 58 | 32 |
| 36 | 0.49 | 95 | 358 | 145 |
| 37 | 0.93 | 102 | 23 | 11 |
| 38 | 1.15 | 82 | 26 | 12 |
| 39 | 0.59 | 113 | 32 | 13 |
| 40 | 0.61 | 91 | 23 | 10 |
| 41 | 0.51 | 98 | 754 | 388 |
| 42 | 0.53 | 105 | 45 | 21 |
| 43 | 0.47 | 82 | 86 | 41 |
| 44 | 0.40 | 95 | 186 | 77 |
| 45 | 0.39 | 97 | 108 | 42 |
| 46 | 0.39 | 98 | 372 | 115 |
| 47 | 0.27 | 55 | 256 | 84 |
| 48 | 0.32 | 71 | 45 | 15 |
| 49 | 0.43 | 123 | 716 | 164 |
| 50 | 0.63 | 87 | 157 | 54 |
| 51 | 0.44 | 117 | 325 | 97 |
| 52 | 0.29 | 98 | 383 | 138 |
| 53 | 0.43 | 109 | 1081 | 273 |
| 54 | 0.76 | 121 | 472 | 131 |
| 55 | 0.33 | 96 | 80 | 49 |
| 56 | 0.21 | 101 | 118 | 61 |
| 57 | 0.08 | 59 | 86 | 44 |
| 58 | 0.35 | 109 | 1171 | 588 |
| 59 | 0.27 | 90 | 434 | 198 |
| 60 | 0.34 | 111 | 487 | 174 |
| 61 | 0.49 | 82 | 1474 | 514 |
| 62 | 0.62 | 111 | 22 | 15 |
| 63 | 1.95 | 135 | 14 | 5 |
| 64 | 0.36 | 100 | 461 | 111 |
| 65 | 0.41 | 96 | 126 | 37 |
| 66 | 0.28 | 102 | 101 | 39 |
| 67 | 0.45 | 120 | 440 | 171 |
| 68 | 0.27 | 95 | 971 | 231 |
| 69 | 0.41 | 110 | 31 | 20 |
| 70 | 0.30 | 91 | 157 | 56 |
| 71 | 1.45 | 130 | 1836 | 593 |
| 72 | 0.37 | 78 | 191 | 69 |
| 73 | 0.40 | 86 | 108 | 62 |
| 74 | 0.44 | 91 | 235 | 109 |
| 75 | 0.91 | 68 | 88 | 21 |
| 76 | 0.46 | 99 | 6085 | 1375 |
| 77 | 0.48 | 102 | 886 | 242 |
| 78 | 0.59 | 144 | 337 | 96 |
| 79 | 0.41 | 100 | 238 | 61 |
| 80 | 0.46 | 108 | 94 | 29 |
| 81 | 0.54 | 112 | 76 | 28 |
| 82 | 0.69 | 120 | 111 | 36 |
| 83 | 0.48 | 97 | 83 | 42 |
| 84 | 0.33 | 94 | 203 | 76 |
| 85 | 1.50 | 93 | 465 | 275 |
| 86 | 0.33 | 102 | 103 | 43 |
| 87 | 0.61 | 121 | 77 | 25 |
| 88 | 0.30 | 69 | 872 | 368 |
| 89 | 0.29 | 102 | 659 | 320 |
| 90 | 0.32 | 110 | 311 | 173 |
| 91 | 0.42 | 103 | 433 | 118 |
| 92 | 0.77 | 129 | 464 | 122 |
| 93 | 0.45 | 113 | 1220 | 431 |
| 94 | 1.85 | 74 | 622 | 273 |
| 95 | 0.76 | 59 | 156 | 40 |
| 96 | 0.41 | 123 | 5421 | 1156 |
| 97 | 0.41 | 125 | 72 | 21 |
| 98 | 0.43 | 132 | 63 | 18 |
| 99 | 0.29 | 99 | 73 | 19 |
| 100 | 0.69 | 104 | 47 | 17 |
| 101 | 0.28 | 92 | 7530 | 936 |
| 102 | 0.28 | 100 | 601 | 229 |
| 103 | 0.57 | 116 | 702 | 112 |
| 104 | 0.36 | 106 | 116 | 23 |
| 105 | 1.20 | 84 | 19 | 6 |
| 106 | 0.53 | 124 | 114 | 28 |
| 107 | 0.65 | 133 | 40 | 10 |
| 108 | 0.72 | 105 | 59 | 17 |
| 109 | 0.32 | 99 | 439 | 110 |
| 110 | 1.15 | 106 | 43 | 14 |
| 111 | 0.28 | 117 | 300 | 76 |
| 112 | 0.48 | 119 | 163 | 39 |
| 113 | 0.71 | 143 | 34 | 7 |
| 114 | 0.39 | 103 | 323 | 128 |
| 115 | 0.42 | 94 | 129 | 32 |
| 116 | 1.10 | 131 | 25 | 16 |
| 117 | 0.44 | 114 | 11 | 3 |
| 118 | 0.55 | 136 | 59 | 29 |
| 119 | 0.35 | 117 | 489 | 303 |
| 120 | 0.38 | 142 | 198 | 65 |
| 121 | 0.42 | 109 | 525 | 372 |
| 122 | 1.20 | 131 | 16 | 5 |
| 123 | 0.38 | 111 | 98 | 31 |
| 124 | 0.44 | 146 | 30 | 11 |
| 125 | 0.47 | 111 | 17 | 6 |
| 126 | 0.97 | 102 | 19 | 6 |
| 127 | >4 | | 85 | 23 |
| 128 | 1.00 | 112 | 41 | 13 |
| 129 | 0.38 | 111 | 45 | 12 |
| 130 | 0.40 | 125 | 34 | 11 |
| 131 | 0.33 | 90 | 37 | 14 |
| 132 | 0.48 | 111 | 20 | 10 |
| 133 | 0.39 | 105 | 104 | 27 |
| 134 | 0.46 | 134 | 29 | 12 |
| 135 | 0.36 | 117 | 299 | 114 |
| 136 | 0.44 | 125 | 974 | 302 |
| 137 | 0.48 | 108 | 116 | 34 |
| 138 | 0.58 | 96 | 41 | 17 |
| 139 | 0.41 | 104 | 39 | 14 |
| 140 | 0.68 | 87 | 103 | 24 |
| 141 | 0.83 | 130 | 107 | 30 |

### Example 7

### In Vivo Studies of Antidiabetic Effect in Db/Db Mice

Acute effect on blood glucose of Compound 1, administered at several dose levels was investigated in db/db mice. The aim of the experiment was to determine the blood glucose lowering effect of the Compound 1 after a single s.c. dose to db/db mice.

### Animals

The mice, 48 male db/db (C57B1/KS db/db), were purchased from Taconic, arriving at Novo Nordisk A/S Animal Unit at 6 weeks of age. The mice were housed according to standard rules in the animal unit and given free access to standard chow and tap water. The animals were kept at a room temperature of 22-24° C. and housed in groups of 5 mice, with free access to food and water, according to Animal Unit's SOP (Housing of experimental animals at Novo Nordisk A/S).

The experiment was carried out two weeks after arrival when the mice were 8 weeks of age.

Study Groups

Group A: Vehicle s.c., injection volume 5 μl/g (n=8) Group C: Compound 1, 10 nmol/kg s.c., injection volume 5 μl/g (n=8) Group D: Compound 1, 30 nmol/kg s.c., injection volume 5 μl/g (n=8)

Group E: Compound 1, 100 nmol/kg s.c., injection volume 5 μl/g (n=8) Group F: Compound 1, 300 nmol/kg s.c., injection volume 5 μl/g (n=8)

### Body Weight

Body weight was measured before the dosing.

### Administration Compounds

Test compound was administered as a single dose s.c.

Injection volume s.c.=5 μL/g

Dose of Compound 1: dose-response 10, 30, 100 and 300 nmol/kg

### Blood Sampling and Analysis

Blood glucose was measured in a 5 μl full blood sample taken from the tip of the tail by puncturing the capillary bed with a lancet, using a 5 μl heparinised capillary tube to sample the blood. The capillary tube was then shaken into 2500 μl glucose/lactate System Solution and measured in a Biosen S_Line, autoanalyser (EKF Diagnostics GmbH, Magdeburg, Germany) according to the manufacturer's instructions. The samples were kept at room temperature until analysis. If analysis was postponed, samples were kept at 4° C. for a maximum of 24 h. The blood samples were taken at the following time points: 0, 30, 60, 120, 180 and 240 minutes and again at 24 h. After the 24 h sample, the blood was sampled from cheek or eye blood for drug exposure measurement in EDTA coated tubes. 50 μl plasma was pipetted from each animal for analysis in an in house immunoassay developed for GLP-1 analogues. The plasma concentrations of the compounds with intact N-terminus were determined using a Luminescence Oxygen Chanelling Immuno-assay (LOCI). The donor beads were coated with streptavidin, while acceptor beads were conjugated with a monoclonal antibody recognizing a mid-/C-terminal epitope of the peptide. The other monoclonal antibody, specific for the N-terminus, was biotinylated. The three reactants were combined with the analyte and formed a two-sited immuno-complex. Illumination of the complex released singlet oxygen atoms from the donor beads, which were channeled into the acceptor beads and triggered chemiluminescence which was measured in an Envision plate reader. The amount of light was proportional to the concentration of the compound.

### Dosing Solutions

Compound 1=55.8 nmol/ml

Compound 1=18.9 nmol/ml

Compound 1=5.1 nmol/ml

Compound 1=1.7 nmol/ml

Vehicle: 50 mM phosphate (Na2HPO4), 145 mM sodium chloride, 50 ppm polysorbate 80 (0.05% Tween 80), pH=7.4

### Data

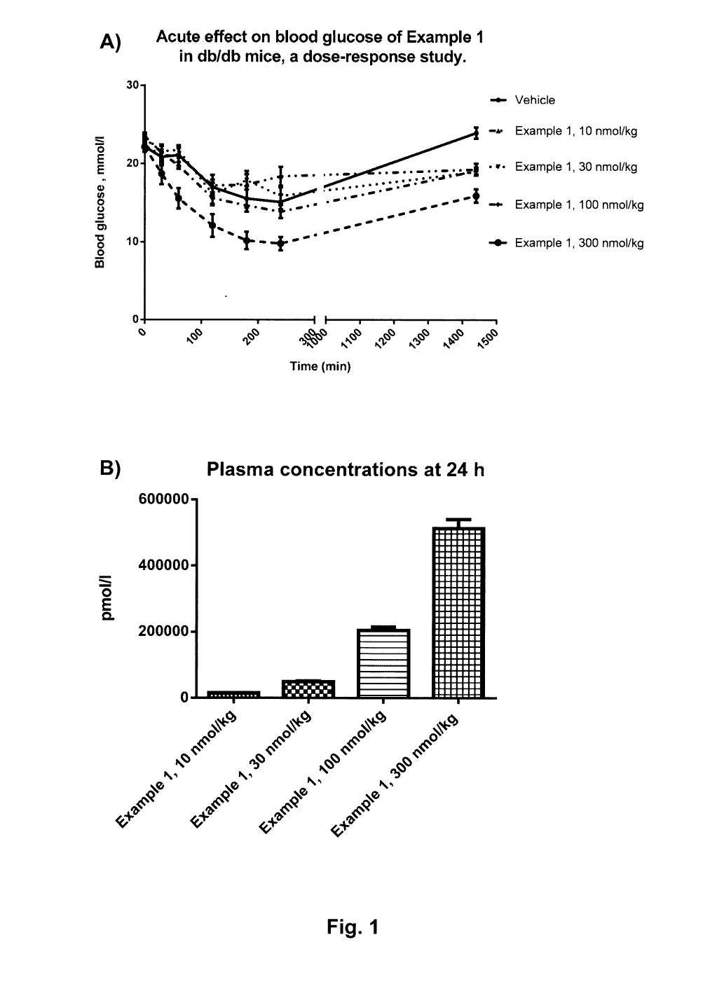

Compound 1 lowered blood glucose in a dose-dependent manner (FIG. 1A). Compound 1 seemed to have duration of action of at least 24 h. Plasma concentration 24 hours after dosing (FIG. 1B) were still very high for Compound 1, indicating a relatively long half-life in plasma of this compound.

### Example 8

### In Vivo Studies on Pharmacokinetics

The aim of the study is to assess the pharmacokinetic properties of the compounds of the invention in normal mice. Two compounds representative of the invention, i.e. Compounds 1 and 5, are subjected to evaluation after intravenous and subcutaneous administration.

### Animals

The mice, 60 female C57B1/6J were purchased from Taconic, arriving at Novo Nordisk A/S Animal Unit at 6-7 weeks of age. The mice were housed according to standard rules in the animal unit and given free access to standard chow and tap water. The animals were kept at a room temperature and housed in groups of 8 mice, with free access to food and water, according to Animal Unit's SOP (Housing of experimental animals at Novo Nordisk A/S).

The experiment was carried out 1 week after arrival when the mice were 7-8 weeks of age.

### Study Groups

Group 1: 15 mice are dosed with Compound 1 subcutaneously (s.c.)

Group 2: 15 mice are dosed with Compound 1 intravenously (i.v.)

Group 3 15 mice are dosed with Compound 5 subcutaneously (s.c.)

Group. 4: 15 mice are dosed with Compound 5 intravenously (i.v.)

All mice are dosed in the morning.

### Body Weight

Body weight was measured before the dosing.

### Administration Peptides and Dosing Solutions

Peptide is administered as a single dose either s.c. or i.v.

Injection volume s.c.=5 μL/g mouse

Injection volume i.v.=5 μL/g mouse

Dose s.c. Compound 1, concentration 64.6 nmol/ml, dose 323 nmol/kg

Dose i.v.: Compound 1, concentration 19.9 nmol/ml, dose 99 nmol/kg

Dose s.c. Compound 5, concentration 12.4 nmol/ml, dose 62 nmol/kg

Dose i.v.: Compound 5, concentration 27.0 nmol/ml, dose 135 nmol/kg

Vehicle: 50 mM phosphate (Na₂HPO₄), 145 mM sodium chloride, 50 ppm polysorbate 80 (0.05% Tween 80), pH=7.4

### Blood Samples and Analysis

Blood samples are taken at 2, 15, 30, 60, 120, 180, 240, 360 min, 24 hours and 30 hours, following a sparse sampling regime, where, 3 samples (=mice) were taken per time point and each mouse had samples taken 2 times from cheek (or eye for the last sample). After the 2^{nd} sample the mice were sacrificed. The plasma concentrations of the compounds with intact N-terminus were determined using a Luminescence Oxygen Chanelling Immuno-assay (LOCI). The donor beads were coated with streptavidin, while acceptor beads were conjugated with a monoclonal antibody recognizing a mid-/C-terminal epitope of the peptide. The other monoclonal antibody, specific for the N-terminus, was biotinylated. The three reactants were combined with the analyte and formed a two-sited immuno-complex. Illumination of the complex released singlet oxygen atoms from the donor beads, which were channeled into the acceptor beads and triggered chemiluminescence which was measured in an Envision plate reader. The amount of light was proportional to the concentration of the compound.

### Data and Results

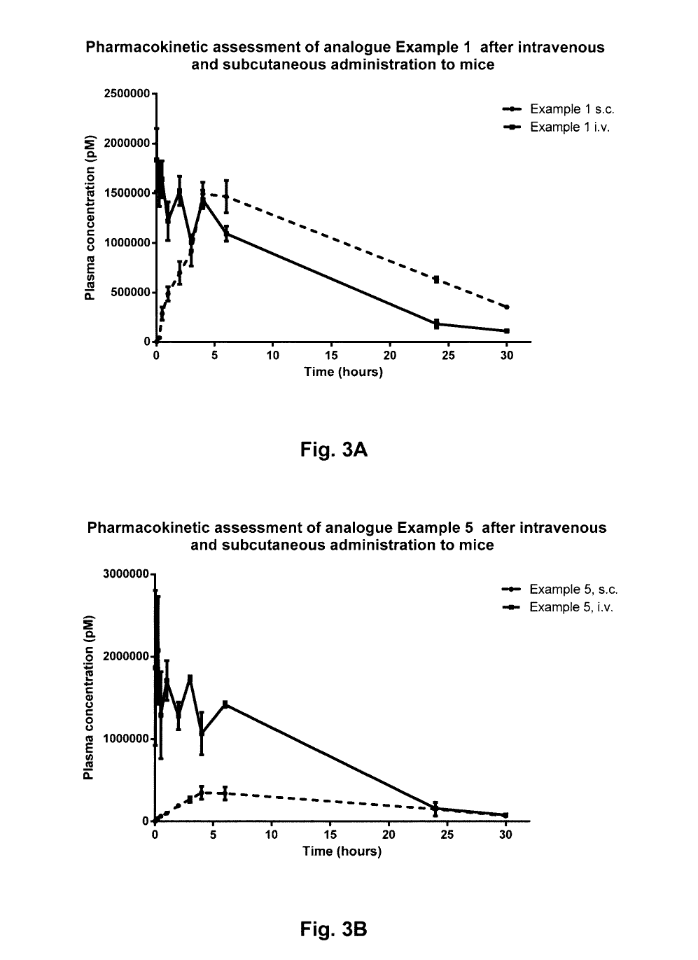

In FIG. 3 the plasma concentration of Compound 1 is displayed as a function of time in both linear and logarithmic scale.

FIG. 3 also display the of the plasma concentration of Compound 5 as a function of time in both linear and logarithmic scale.

The pharmacokinetic data are summarised in Tables 7 and 8 (mean values only).

TABLE 7
In vivo studies on pharmacokinetic evaluation of Compound
1 and Compound 5 in mice after intravenous administration

| | | | | | Volume of |
|---|---|---|---|---|---|
| Compound | | T_{1/2} | Conc. at 0 h | Clearance | distribution |
| No. | RoA | (hours) | (pmol/l) | (l/kg) | (l/kg) |
| 1 | i.v. | 6.7 | 2094252 | 0.004686 | 0.045604 |
| 5 | i.v. | 5.9 | 1862933 | 0.005603 | 0.047986 |

TABLE 8
In vivo studies on pharmacokinetic evaluation of Compound 1
and Compound 5 in mice after subcutaneous administration

| Com- | | | | | | Volume of | |
|---|---|---|---|---|---|---|---|
| pound | | T_{1/2} | Cₘₐₓ | Tₘₐₓ | Clearance | distribution | |
| No | RoA | (hours) | pmol/l) | (h) | (l/h/kg) | (l/kg) | % |
| 1 | s.c. | 11.5 | 1493333 | 4 | 0.009567 | 0.159180 | 49.0 |
| 5 | s.c. | 9.7 | 348667 | 4 | 0.008298 | 0.116427 | 67.5 |

### Example 9

### In Vivo Studies on Pharmacokinetics

The aim of the study is to assess the pharmacokinetic properties of the compounds of the invention, in mini-pigs. Six compounds representative of the invention, i.e. Compounds 1, 4, 5, 6, 7 and 8, were subjected to evaluation after intravenous administration.

### Animals

Göttingen mini-pigs female, 15-20 kg, purchased from Ellegaard Mini-pigs, Denmark, were housed in the Animal Unit, Novo Nordisk A/S and were kept and handled according to normal procedure in the Animal Unit. After minimum 2 weeks of acclimatization two permanent central venous catheters were implemented in vena cava caudalis in each animal. After surgery the animals were in their normal individual pens during the pharmacokinetic experiments.

### Body Weight

The animals were weighed weekly. The animals were fasted on the morning prior to dosing but had ad libitum access to water; food was supplied during dosing.

### Administration of Peptides and Dosing Solutions

Intravenous injections were given through the central short catheter, which was flushed with min 10 ml of sterile saline post administration. The test substance was dosed at 5 nmol/kg, N=3, in a volume of 0.05 ml/kg.

Buffer: 20 mM phosphate, 130 mM sodium chloride, 0.05% Tween 80, pH=7.4

| Test substance | Concentration |
|---|---|
| (Compound No.) | (nmol/ml) |
| 1 | 92.3 |
| 4 | 99.9 |
| 5 | 97.5 |
| 6 | 98 |
| 7 | 95 |
| 8 | 95.5 |

### Blood Samples and Analysis

Blood samples were taken through the central catheter according to the following schedule: Predose, 5, 15, 30, 45 min, 1 h, 1.5 h, 2 h, 3 h, 4 h, 6 h, 8 h, 10 h, 24 h, 48 h, 72 h,96 h, 120 h, 168 h, 216 h, 240 h, 264 h and 288 h. On day 1 the catheters are coupled to extension tubes, which will be removed at the end of day 1.

Samples (0.8 ml) were taken through the catheter. Blood was collected in test tubes containing EDTA buffer (8 mM) for stabilisation. After each blood sample the catheter was flushed with min 5 ml of sterile 0.9% NaCl and 10 IE/mI heparin. Aseptic technique was demanded to avoid bacterial growth in the catheter with increased risk of clotting.

Samples were kept on wet ice until centrifugation (10 min, 4° C., 1942 g). Afterwards, plasma (min. 100 μl) was transferred immediately to Micronic tubes and kept at −20° C. until analysis. The plasma samples were analysed by an immunoaasay described below. The LLOQ was 500-1500 μM.

The plasma concentrations of the peptides with intact N-terminus were determined using a Luminescence Oxygen Chanelling Immuno-assay (LOCI). The donor beads were coated with streptavidin, while acceptor beads were conjugated with a monoclonal antibody recognizing a mid-/C-terminal epitope of the peptide. The other monoclonal antibody, specific for the N-terminus, was biotinylated. The three reactants were combined with the analyte and formed a two-sited immuno-complex. Illumination of the complex released singlet oxygen atoms from the donor beads, which were channeled into the acceptor beads and triggered chemi-luminescence which was measured in an Envision plate reader. The amount of light was proportional to the concentration of the compound.

### Data and Results

Plasma concentration-time profiles was analysed by a non-compartmental pharmacokinetics analysis using Phoenix (Pharsight Inc., Mountain View, Calif., USA). Calculations were performed using individual concentration-time values from each animal.

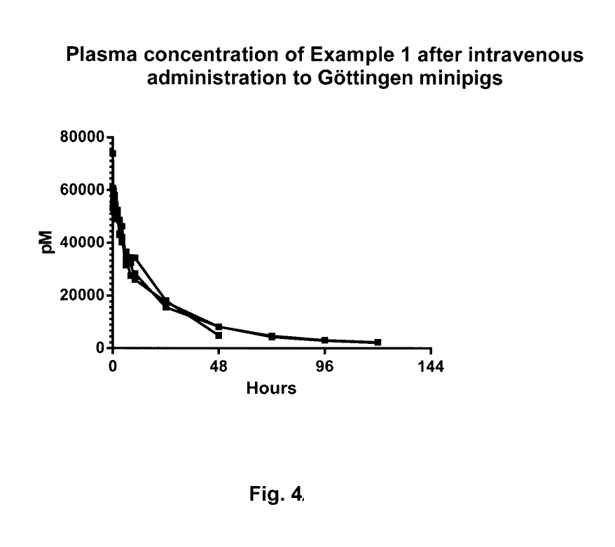

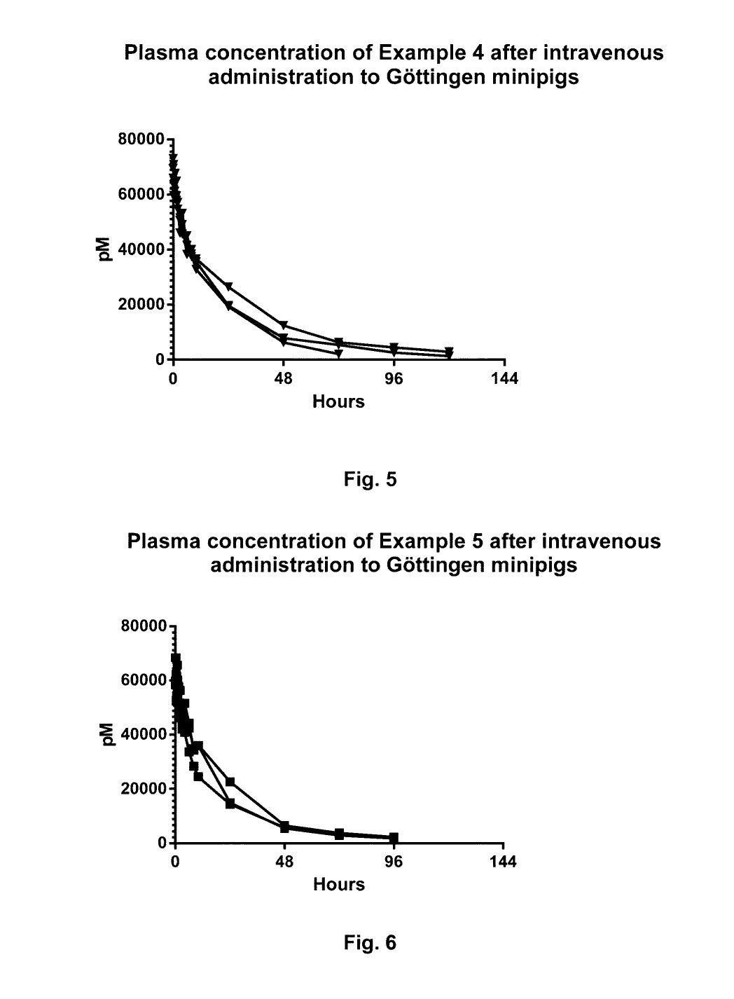

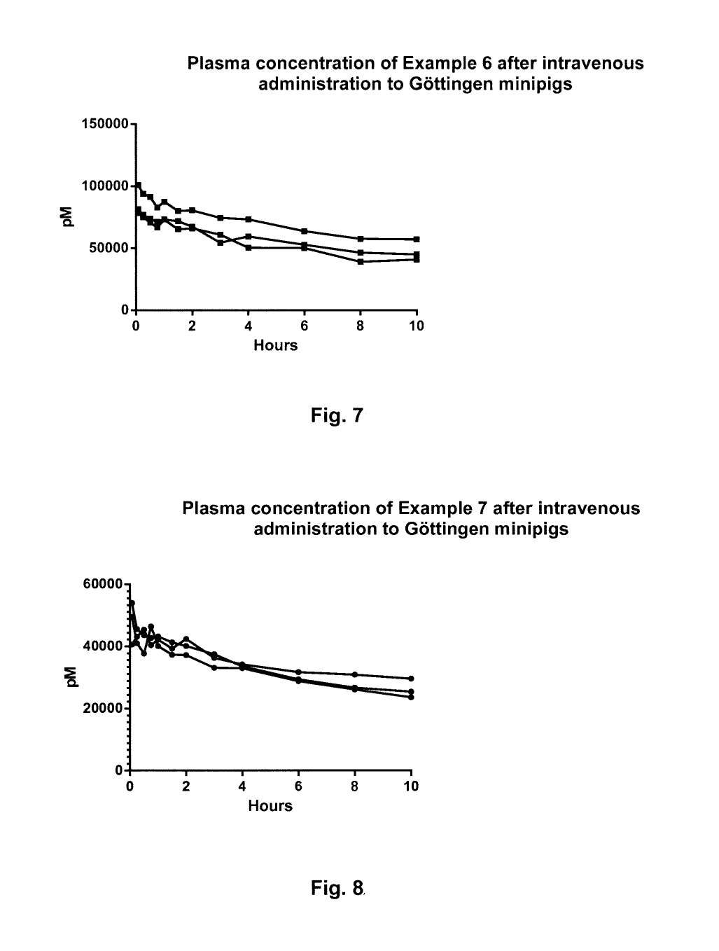

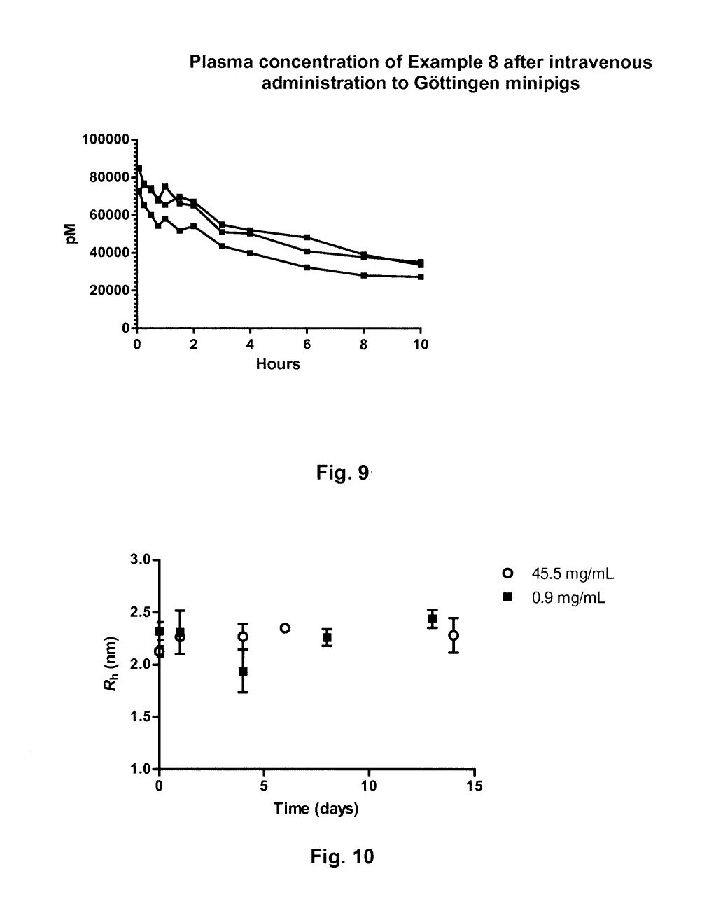

The T_{1/2} was 33.7, 20.6, 23.0, 38.7, 27.3 and 32.8 hours, respectively, for Compound 1, Compound 4, Compound 5, Compound 6, Compound 7 and Compound 8. Table 9 summarizes the data from the experiments. FIGS. 4-9 display the plasma concentrations as a function of time, displayed on linear and logarithmic scales, respectively.

TABLE 9
In vivo studies on pharmacokinetic evaluation of some
compounds representative of the invention in Göttingen
mini-pigs after intravenous administration

| | | | | Volume of |
|---|---|---|---|---|
| | C0 | T_{1/2} | Clearance | Distribution |
| Compound | (pmol/l) | (h) | (l/h/kg) | (l/kg) |
| No. | Mean Value | Mean Value | Mean Value | Mean Value |
| 1 | 57226 | 33.7 | 0.003295 | 0.160233 |
| 4 | 72094 | 20.6 | 0.003311 | 0.100274 |
| 5 | 63272 | 23.0 | 0.003871 | 0.128244 |
| 6 | 89499 | 38.7 | 0.001640 | 0.090428 |
| 7 | 51231 | 27.3 | 0.003705 | 0.145576 |
| 8 | 79676 | 32.9 | 0.003091 | 0.151675 |
| 11 | 114619 | 0.8 | 0.054855 | 0.061337 |
| 16 | 123149 | 42.8 | 0.001439 | 0.088881 |
| 26 | 122525 | 7.3 | 0.005212 | 0.054918 |
| 32 | 108124 | 51.3 | 0.001038 | 0.076859 |
| 45 | 147085 | 44.9 | 0.002386 | 0.154374 |
| 48 | 85458 | 8.3 | 0.006303 | 0.074285 |
| 49 | 131167 | 24.0 | 0.002680 | 0.087791 |
| 54 | 135705 | 40.7 | 0.001315 | 0.080192 |
| 61 | 53271 | 31.8 | 0.005899 | 0.270045 |
| 62 | 50607 | 16.7 | 0.002658 | 0.065388 |
| 64 | 86933 | 25.2 | 0.002411 | 0.087908 |
| 67 | 163171 | 33.0 | 0.001172 | 0.056037 |
| 76 | 135596 | 41.7 | 0.001057 | 0.063622 |
| 78 | 58365 | 20.6 | 0.004447 | 0.133991 |
| 87 | 154794 | 19.0 | 0.003469 | 0.095772 |
| 99 | 95771 | 53.2 | 0.001199 | 0.092087 |
| 107 | 59159 | 5.4 | 0.008036 | 0.061467 |
| 118 | 62866 | 11.2 | 0.004987 | 0.080915 |

1. A GLP-1 receptor agonist peptide which in an alpha helical conformation comprise an amphipathic helix, wherein said peptide has cholesterol efflux activity with an Eₘₐₓ of at least 65% of that of L-4F, and a GLP-1 receptor potency measured as EC₅₀, that is better than the potency of L-4F, when measured according to the methods described in Example 6.

2. The GLP-1 receptor agonist peptide of claim 1, wherein said peptide comprises at least 31 amino acid residues.

3. The GLP-1 receptor agonist peptide of claim 1, wherein said amphipathic helix comprises at least 15 amino acid residues.

4. The GLP-1 receptor agonist peptide of claim 1, wherein said amphipathic helix comprises a hydrophilic and a lipophilic face.

5. The GLP-1 receptor agonist peptide of claim 4, wherein said hydrophilic face comprises at least 6 amino acid residues, wherein at least 4 amino acid residues are charged.

6. The GLP-1 receptor agonist peptide of claim 4, wherein said lipophilic face comprises at least 7 amino acid residues, wherein at least 6 amino acid residues are lipophilic.

7. The GLP-1 receptor agonist peptide according to claim 1, comprising a peptide with a sequence identity of more than 90% when compared to the sequences of SEQ ID 9, SEQ ID 10, or SEQ ID 11, and having up to 3 additional Aib substitutions.

8. The GLP-1 receptor agonist peptide according to claim 1, which is a GLP-1 receptor agonist peptide comprising an amino acid sequence of Formula I:
X₇-X₈-X₉-Gly-Thr-X₁₂-Thr-X₁₄-Asp-X₁₆-X₁₇-X₁₈-X₁₉-X₂₀-X₂₁-X₂₂-X₂₃-X₂₄-X₂₅-X₂₆-X₂₇-Phe-X₂₉-X₃₀-X₃₁-Leu-X₃₃-X₃₄-X₃₅-X₃₆-X₃₇-X₃₈-X₃₉-X₄₀-X₄₁-X₄₂-X₄₃-X₄₄-X₄₅-X₄₆-X₄₇-X₄₈-X₄₉-X₅₀
wherein
X₇ represents His, or desamino-His;
X₈ represents Ala, Gly, Ser, or Aib;
X₉ represents Glu, Asp, Gln, or His;
X₁₂ represents Phe, Tyr, or Leu;
X₁₄ represents Ser, Asn, or His;
X₁₆ represents Val, Tyr, Leu, Ile, or Met;
X₁₇ represents Ser, or Thr;
X₁₈ represents Ser, Lys, Arg, Glu, Asn, or Gln;
X₁₉ represents Tyr, or Gln;
X₂₀ represents Leu, Met, or Tyr;
X₂₁ represents Glu, Asp, or Gln;
X₂₂ represents Gly, Ser, Glu, Lys, Aib, or Pro;
X₂₃ represents Gln, Glu, Lys, Trp, Arg, or Asp;
X₂₄ represents Ala, Aib, Lys, or Arg;
X₂₅ represents Ala, Val, Phe, His, Leu, Met, Trp, Tyr, Ile, or Aib;
X₂₆ represents Lys, Asn, Glu, Arg, His, Gly, Val, or Gln;
X₂₇ represents Glu, Asp, Gln, Ala, His, Gly, Arg, Lys, Aib, or Leu;
X₂₉ represents Ile, or Val;
X₃₀ represents Ala, Val, Gln, Ile, Trp, Aib, Glu, Arg, or Lys;
X₃₁ represents Trp, Gln, Lys, or His;
X₃₃ represents Val, Met, Ile, Leu, Thr, Arg, or Lys;
X₃₄ represents Lys, Glu, Asn, Asp, Gln, His, Gly, or Arg;
X₃₅ represents Gly, Lys, Arg, His, Ser, Thr, Aib, Ala, or Gln;
X₃₆ represents Gly, Aib, Val, Leu, Ala, His, Ile, Met, Trp, Tyr, or Phe;
X₃₇ represents Gly, Ala, Glu, Aib, His, Arg, Leu, Pro, Lys, or Gln;
X₃₈ represents Glu, Ser, Asp, His, Gly, Gln, or amide, or X₃₈ is absent;
X₃₉ represents Phe, Leu, His, Ala, Ser, Ile, Met, Val, Trp, Tyr, Gly, Glu, Lys, or amide, or
X₃₉ is absent;
X₄₀ represents Leu, Phe, Val, His, Gly, Ala, Ile, Met, Trp, Tyr, or amide, or X₄₀ is absent;
X₄₁ represents Glu, Asp, Ala, Gly, Lys, or amide, or X₄₁ is absent;
X₄₂ represents Leu, Pro, Lys, Arg, or amide, or X₄₂ is absent;
X₄₃ represents Leu, Pro, Val, or amide, or X₄₃ is absent;
X₄₄ represents Lys, or amide, or X₄₄ is absent;
X₄₅ represents Glu, or amide, or X₄₅ is absent;
X₄₆ represents Phe, Ile, or amide, or X₄₆ is absent;
X₄₇ represents Ile, or amide, or X₄₇ is absent;
X₄₈ represents Ala, or amide, or X₄₈ is absent;
X₄₉ represents Trp, or amide, or X₄₉ is absent;
X₅₀ represents amide, or X₅₀ is absent;
with the proviso that if X₃₈, X₃₉, X₄₀, X₄₁, X₄₂, X₄₃, X₄₄, X₄₅, X₄₆, X₄₇, X₄₈, X₄₉ or X₅₀ is absent, then each amino acid residue downstream is also absent;
or a pharmaceutically acceptable salt, amide, ester, or acid, or a prodrug thereof.

9. The GLP-1 receptor agonist peptide according to claim 8, wherein X₇-X₃₅ represents Exendin-4(1-29), GLP-1(7-35), or glucagon peptide (1-29), with up to 12 amino acid substitutions.

10. A GLP-1 receptor agonist peptide comprising an amino acid sequence of Formula I:
X₇-X₈-X₉-Gly-Thr-X₁₂-Thr-X₁₄-Asp-X₁₆-X₁₇-X₁₈-X₁₉-X₂₀-X₂₁-X₂₂-X₂₃-X₂₄-X₂₅-X₂₆-X₂₇-Phe-X₂₉-X₃₀-X₃₁-Leu-X₃₃-X₃₄-X₃₅-X₃₆-X₃₇-X₃₈-X₃₉-X₄₀-X₄₁-X₄₂-X₄₃-X₄₄-X₄₅-X₄₆-X₄₇-X₄₈-X₄₉-X₅₀
wherein
X₇ represents His, or desamino-His;
X₈ represents Ala, Gly, Ser, or Aib;
X₉ represents Glu, Asp, Gln, or His;
X₁₂ represents Phe, Tyr, or Leu;
X₁₄ represents Ser, Asn, or His;
X₁₆ represents Val, Tyr, Leu, Ile, or Met;
X₁₇ represents Ser, or Thr;
X₁₈ represents Ser, Lys, Arg, Glu, Asn, or Gln;
X₁₉ represents Tyr, or Gln;
X₂₀ represents Leu, Met, or Tyr;
X₂₁ represents Glu, Asp, or Gln;
X₂₂ represents Gly, Ser, Glu, Pro, Lys, or Aib;
X₂₃ represents Gln, Glu, Lys, Trp, or Asp;
X₂₄ represents Ala, Aib, Lys, or Arg;
X₂₅ represents Ala, Val, Leu, Ile, or Aib;
X₂₆ represents Lys, Asn, Glu, Arg, His, Gly, Val, or Gln;
X₂₇ represents Glu, Asp, Gln, Ala, His, Gly, Arg, Lys, Aib, or Leu;
X₂₉ represents Ile, or Val;
X₃₀ represents Ala, Val, Gln, Ile, Trp, Aib, Glu, Arg, or Lys;
X₃₁ represents Trp, Gln, Lys, or His;
X₃₃ represents Val, Ile, Leu, Thr, Arg, or Lys;
X₃₄-X₃₅-X₃₆-X₃₇-X₃₈-X₃₉ represents Subsequence 1, composed by the following amino acid residues “Glu-Lys-Aib-Lys-Glu-Phe”; or in which Subsequence 1, one, two or three amino acid residues have been substituted for
Asn, Gln, Lys, His, Gly, Arg, or Asp in position X₃₄;
Arg, Ala, His, Gln, Asn, or Aib in position X₃₅;
Gly, Val, Leu, Phe, Ile, Trp, Tyr, Ala, Met, or His in position X₃₆;
Arg, Ala, Leu, Gly, His, Gln, Asn, Aib, Ile, Val, or Phe in position X₃₇;
Asp, His, Gln, Ser, Gly, Asn, or Thr in position X₃₈; and/or
Trp, Ala, Glu, Leu, Val, Gly, His, Lys, Ser, Thr, Tyr, Aib, Ile, or Met in position X₃₉; and
X₄₀ represents Leu, Phe, Val, His, Tyr, or amide, or X₄₀ is absent;
X₄₁ represents Glu, Asp, Ala, Gly, Lys, or amide, or X₄₁ is absent;
X₄₂ represents Leu, Pro, Lys, Arg, or amide, or X₄₂ is absent;
X₄₃ represents Leu, Pro, Val, or amide, or X₄₃ is absent;
X₄₄ represents Lys, or amide, or X₄₄ is absent;
X₄₅ represents Glu, or amide, or X₄₅ is absent;
X₄₆ represents Phe, Ile, or amide, or X₄₆ is absent;
X₄₇ represents Ile, or amide, or X₄₇ is absent;
X₄₈ represents Ala, or amide, or X₄₈ is absent;
X₄₉ represents Trp, or amide, or X₄₉ is absent;
X₅₀ represents amide, or X₅₀ is absent;
provided, however:
if X₄₁, X₄₂, X₄₃, X₄₄, X₄₅, X₄₆, X₄₇, X₄₈, X₄₉ or X₅₀ is absent, then each amino acid residue downstream is also absent;
and pharmaceutically acceptable salts, amides, esters, acids or prodrugs thereof.

11. The GLP-1 receptor agonist peptide according to claim 1, said peptide having up to 45 amino acid residues and comprising the amino acid sequence of SEQ ID 9, with up to 10 conservative mutations.

12. A The GLP-1 receptor agonist peptide according to claim 1, said peptide having up to 45 amino acid residues and comprising the amino acid sequence of SEQ ID 10, with up to 10 conservative mutations.

13. A The GLP-1 receptor agonist peptide according to claim 1, said peptide having up to 45 amino acid residues and comprising the amino acid sequence of SEQ ID 11, with up to 10 conservative mutations.

14. A GLP-1 receptor agonist peptide selected from the group consisting of:
[Aib8,Glu23,Val25,Glu34,Lys35,Aib36,Lys37]-GLP-1-(7-37)-peptidyl-Glu-Phe-Leu amide;
[Aib2,Gly16,Glu21,Lys29,Aib30,Leu31,Glu32,Phe33,Leu34,Glu35,Leu36,Leu37]-Exendin-4-(1-37)-peptide amide;
[Aib8,Glu23,Aib24,Val25,Aib30,Glu34,Lys35,Aib36,Lys37]-GLP-1-(7-37)-peptidyl-Glu-Phe-Leu amide;
[Aib8,Glu23,Val25,Glu34,Lys35,Aib36,Lys37]-GLP-1-(7-37)-peptidyl-Glu-His-Leu amide;
[Aib8,Glu23,Val25,Glu34,Lys35,Val36,Lys37]-GLP-1-(7-37)-peptidyl-Glu-His-Leu amide;
[Aib8,Glu23,Val25,Glu34,Lys35,Val36,Lys37]-GLP-1-(7-37)-peptidyl-Glu-Phe-Leu amide;
[Aib8,Val25,Glu34,Lys35,Aib36,Lys37]-GLP-1-(7-37)-peptidyl-Glu-Phe-Leu amide;
[Aib8,Glu23,Glu34,Lys35,Aib36,Lys37]-GLP-1-(7-37)-peptidyl-Glu-Phe-Leu amide;
[Aib8,Glu23,Val25,Lys35,Aib36,Lys37]-GLP-1-(7-37)-peptidyl-Glu-Phe-Leu amide;
[Aib8,Glu23,Val25,Aib36,Lys37]-des-Lys34-GLP-1-(7-37)-peptidyl-Glu-Phe-Leu amide;
[Aib8,Glu23,Val25,Glu34,Lys35,Aib36,Lys37]-GLP-1-(7-37)-peptide amide;
[Aib2,Gly16,Lys29,Aib30,Leu31,Glu32,Phe33,Leu34,Glu35,Leu36,Leu37]-Exendin-4-(1-37)-peptide amide;
[Tyr12,Asn14,Thr17,Glu18,Glu23,Val25,Glu34,Lys35,Aib36,Lys37]-GLP-1-(7-37)-peptidyl-Glu-Phe-Leu amide;
[Asp9,Leu12,Ile16,Glu23,Val25,Glu34,Lys35,Aib36,Lys37]-GLP-1-(7-37)-peptidyl-Glu-Phe-Leu amide;
[His14,Tyr20,Glu23,Val25,Glu34,Lys35,Aib36,Lys37]-GLP-1-(7-37)-peptidyl-Glu-Phe-Leu amide;
[Aib8,Glu23,Val25,Glu34,Lys35,Aib36,Lys37]-GLP-1-(7-37)-peptidyl-Glu-Phe-Leu;
[Aib8,Glu23,Val25,His31,Glu34,Lys35,Aib36,Lys37]-GLP-1-(7-37)-peptidyl-Glu-Phe-Leu amide;
[Aib8,Asp23,Val25,Asp27,Glu34,Lys35,Aib36,Lys37]-GLP-1-(7-37)-peptidyl-Glu-Phe-Leu amide;
[Aib8,Glu23,Val25,Arg26,Glu34,Arg35,Aib36,Lys37]-GLP-1-(7-37)-peptidyl-Glu-Phe-Leu amide;
[Aib8,Glu23,Val25,Glu34,Lys35,Leu36,Lys37]-GLP-1-(7-37)-peptidyl-Glu-Leu-Val amide;
[Aib8,Trp23,Val25,Glu34,Lys35,Aib36,Lys37]-GLP-1-(7-37)-peptidyl-Glu-Phe-Leu amide;
[Aib8,Glu23,Val25,Glu34,Lys35,Aib36,Lys37]-GLP-1-(7-37)-peptidyl-Glu-Phe-His amide;
[Aib2,Glu21,Lys29,Aib30,Leu31,Glu32,Phe33,Leu34,Glu35,Leu36,Leu37]-Exendin-4-(1-37)-peptide;
[Aib2,Gly16,Lys17,Ala18,Arg20,Glu21,Leu27,Glu28,Lys29]-Glucagonyl-(1-29)-Aib-Lys-Glu-Phe-Leu amide;
[Aib2,Gly16,Lys17,Arg20,Glu21,Leu27,Glu28,Lys29]-Glucagonyl-(1-29)-Aib-Lys-Glu-Phe-Leu amide;
[Aib2,Lys17,Ala18,Arg20,Glu21,Leu27,Glu28,Lys29]-Glucagonyl-(1-29)-Aib-Lys-Glu-Phe-Leu amide;
[Asn14,Met16,Thr17,Asn18,Glu23,Val25,Glu34,Lys35,Gly36,Lys37]-GLP-1-(7-37)-peptidyl-Glu-Phe-Leu amide;
[Aib8,Glu23,Val25,Leu27,Glu34,Lys35,Aib36,Lys37]-GLP-1-(7-37)-peptidyl-Glu-Phe-Leu amide;
[Aib2,Glu21,Lys29,Aib30,Leu31,Glu32,Phe33,Leu34,Glu35,Leu36,Leu37]-Exendin-4-(1-37)-peptide amide;
[Aib8,Glu23,Val25,Glu34,Aib36,Lys37]-GLP-1-(7-37)-peptidyl-Glu-Phe-Leu amide;
[Aib8,Glu23,Val25,Glu34,Lys35,Aib36,Lys37]-GLP-1-(7-37)-peptidyl-His-Phe-Leu amide;
[Aib8,Glu23,Val25,Glu34,Lys35,Aib36]-GLP-1-(7-37)-peptidyl-Glu-Phe-Leu amide;
[Aib2,Glu21,Lys29,Leu31,Glu32,Phe33,Leu34,Glu35,Leu36,Leu37]-Exendin-4-(1-37)-peptide amide;
[Aib2,Glu21,Aib30,Leu31,Glu32,Phe33,Leu34,Glu35,Leu36,Leu37]-Exendin-4-(1-37)-peptide amide;
[Aib2,Glu21,Lys29,Aib30,Glu32,Phe33,Leu34,Glu35,Leu36,Leu37]-Exendin-4-(1-37)-peptide amide;
[Aib2,Glu21,Lys29,Aib30,Leu31,Phe33,Leu34,Glu35,Leu36,Leu37]-Exendin-4-(1-37)-peptide amide;
[Aib2,Glu21,Lys29,Aib30,Leu31,Glu32,Leu34,Glu35,Leu36,Leu37]-Exendin-4-(1-37)-peptide amide;
[Aib2,Glu21,Lys29,Aib30,Leu31,Glu32,Phe33,Glu35,Leu36,Leu37]-Exendin-4-(1-37)-peptide amide;
[Aib2,Glu21,Lys29,Aib30,Leu31,Glu32,Phe33,Leu34,Glu35,Leu37]-Exendin-4-(1-37)-peptide amide;
[Aib2,Glu21,Lys29,Aib30,Leu31,Glu32,Phe33,Leu34,Glu35,Leu36]-Exendin-4-(1-37)-peptide amide;
[Aib2,Glu21,Lys29,Aib30,Leu31,Glu32,Phe33,Leu34,Leu36,Leu37]-Exendin-4-(1-37)-peptide amide;
[Aib2,Glu21,Lys29,Aib30,Leu31,Glu32,Phe33,Leu34]-Exendin-4-(1-34)-peptide amide;
[Aib8,Glu23,Val25,Arg26,Glu34,Arg35,Aib36,Arg37]-GLP-1-(7-37)-peptidyl-Glu-Phe-Leu amide;
[Asn14,Met16,Thr17,Asn18,Glu23,Val25,Glu34,Lys35,Aib36,Lys37]-GLP-1-(7-37)-peptidyl-Glu-Phe-Leu amide;
[Aib8,Glu34,Lys35,Aib36,Lys37]-GLP-1-(7-37)-peptidyl-Glu-Phe-Leu amide;
[Aib8,Glu23,Val25,Glu34,Lys35,Phe36,Lys37]-GLP-1-(7-37)-peptidyl-Glu-Ala-Phe amide;
[Aib8,Lys35,Aib36,Lys37]-GLP-1-(7-37)-peptidyl-Glu-Phe-Leu amide;
[Aib8,Lys35,Aib36]-GLP-1-(7-37)-peptidyl-Glu-Phe-Leu amide;
[Aib8,Glu23,Lys24,Val25,Glu30,Glu34,Lys35,Aib36,Lys37]-GLP-1-(7-37)-peptidyl-Glu-Phe-Leu amide;
[Aib8,Glu23,Val25,Trp30,Glu34,Lys35,Aib36,Lys37]-GLP-1-(7-37)-peptidyl-Glu-Phe-Leu amide;
[Aib8,Glu23,Val25,Lys27,Glu34,Lys35,Aib36,Lys37]-GLP-1-(7-37)-peptidyl-Glu-Phe-Leu amide;
[Aib8,Lys23,Arg24,Arg26,Glu34,Lys35,Aib36,Lys37]-GLP-1-(7-37)-peptidyl-Glu-Phe-Leu amide;
[Aib8,Glu23,Arg24,Val25,Glu34,Lys35,Aib36,Lys37]-GLP-1-(7-37)-peptidyl-Glu-Phe-Leu amide;
[Aib8,Glu23,Val25,Glu34,Lys35,Aib36,Lys37]-GLP-1-(7-37)-peptidyl-Glu-Phe-Leu-Glu-Lys-Val amide;
[Aib2,His3,Glu15,Glu16,Glu17,Ala18,Lys20,Glu21,Ile23,Ala24,Leu27,Glu28,Lys29]-Glucagonyl-(1-29)-Aib-Lys-Glu-Phe-Leu amide;
[Glu15,Glu16,Gln17,Ala18,Lys20,Glu21,Ile23,Ala24,Leu27,Glu28,Lys29]-Glucagonyl-(1-29)-Aib-Lys-Glu-Phe-Leu amide;
[Glu15,Glu16,Lys17,Ala18,Lys20,Glu21,Ile23,Ala24,Leu27,Glu28,Lys29]-Glucagonyl-(1-29)-Aib-Lys-Glu-Phe-Leu amide;
[Glu15,Glu16,Lys17,Lys18,Lys20,Glu21,Ile23,Ala24,Leu27,Glu28,Lys29]-Glucagonyl-(1-29)-Aib-Lys-Glu-Phe-Leu amide;
[Lys17,Ala18,Arg20,Glu21,Leu27,Glu28,Lys29]-Glucagonyl-(1-29)-Aib-Lys-Glu-Phe-Leu amide;
[Aib8,Glu23,Lys24,Val25,Glu34,Lys35,Aib36,Lys37]-GLP-1-(7-37)-peptidyl-Glu-Phe-Leu amide;
[Aib8,Glu23,Val25,Glu34,Lys35,Aib36,Lys37]-GLP-1-(7-37)-peptidyl-Glu-Phe-Leu-Glu-Lys-Val-Lys-Glu-Phe amide;
[Aib8,Glu23,Val25,Glu34,Lys35,Aib36,Lys37]-GLP-1-(7-37)-peptidyl-Glu-Phe amide;
[Aib8,Glu23,Val25,Glu34,Lys35,Aib36,Lys37]-GLP-1-(7-37)-peptidyl-Glu amide;
[Aib8,Glu23,Lys24,Val25,Glu34,Aib36,Lys37]-GLP-1-(7-37)-peptidyl-Glu-Phe-Leu amide;
[Aib8,Glu23,Val25,Val29,Gln30,Glu34,Lys35,Aib36,Lys37]-GLP-1-(7-37)-peptidyl-Glu-Phe-Leu amide;
[Aib8,Glu23,Val25,Glu34,Lys35,Aib36,Lys37]-GLP-1-(7-37)-peptidyl-Glu-Phe-Leu-Glu amide;
[Glu23,Val25,Glu34,Lys35,Val36,Lys37]-GLP-1-(7-37)-peptidyl-Glu-Phe-Leu amide;
[Ala2,Lys17,Ala18,Arg20,Glu21,Leu27,Glu28,Lys29]-Glucagonyl-(1-29)-Val-Lys-Glu-Phe-Leu amide;
[Ala2,Glu21,Lys29,Va130,Leu31,Glu32,Phe33,Leu34]-Exendin-4-(1-34)-peptide amide;
[Aib8,Glu23,Val25,Glu34,Lys35,Aib36,Lys37]-GLP-1-(7-37)-peptidyl-Glu-Phe-Leu-Glu-Lys amide;
[Aib8,Glu23,Lys24,Val25,Glu30,His31,Glu34,Lys35,Aib36,Lys37]-GLP-1-(7-37)-peptidyl-Glu-Phe-Leu amide;
[Aib8,Val25,Gln27,Glu34,Lys35,Aib36,Ala37]-GLP-1-(7-37)-peptidyl-Glu-Phe-Leu amide;
[Aib8,Val25,Asn34,Lys35,Aib36,Ala37]-GLP-1-(7-37)-peptidyl-Glu-Phe-Leu amide;
[Aib8,Val25,Gln27,Asn34,Lys35,Aib36,Ala37]-GLP-1-(7-37)-peptidyl-Glu-Phe-Leu amide;
[Aib8,Val36]-GLP-1-(7-37)-peptidyl-Glu-Phe-Leu amide;
[Gly8,Glu23,Val25,Glu34,Lys35,Val36,Lys37]-GLP-1-(7-37)-peptidyl-Glu-Phe-Leu amide;
[desamino-His7,Aib8,Glu23,Val25,Glu34,Lys35,Val36,Lys37]-GLP-1-(7-37)-peptidyl-Glu-Phe-Leu amide;
[Gly8,Glu23,Val25,Glu34,Lys35,Val36,Lys37]-GLP-1-(7-37)-peptidyl-Glu-His-Leu amide;
[Aib8,Glu23,Val25,Gln30,Leu33,Glu34,Lys35,Aib36,Lys37]-GLP-1-(7-37)-peptidyl-Glu-Phe-Leu amide;
[Aib8,Glu23,Val25,Arg26,Val29,Gln30,Leu33,Glu34,Lys35,Aib36,Lys37]-GLP-1-(7-37)-peptidyl-Glu-Phe-Leu amide;
[Aib8,Glu23,Val25,Arg26,Glu34,Lys35,Aib36,Lys37]-GLP-1-(7-37)-peptidyl-Glu-Phe-Leu amide;
[Aib8,Glu23,Val25,Val29,Glu34,Lys35,Aib36,Lys37]-GLP-1-(7-37)-peptidyl-Glu-Phe-Leu amide;
[Aib8,Glu23,Val25,Gln30,Glu34,Lys35,Aib36,Lys37]-GLP-1-(7-37)-peptidyl-Glu-Phe-Leu amide;
[Aib8,Glu23,Val25,Leu33,Glu34,Lys35,Aib36,Lys37]-GLP-1-(7-37)-peptidyl-Glu-Phe-Leu amide;
[Aib8,Glu23,Val25,Lys30,Glu34,Lys35,Leu36,Lys37]-GLP-1-(7-37)-peptidyl-Glu-Phe-Leu amide;
[Aib8,Glu23,Val25,Lys30,Glu34,Lys35,Aib36,Lys37]-GLP-1-(7-37)-peptidyl-Glu-Phe-Leu amide;
[Aib8,Glu23,Val25,Gln30,His31,Glu34,Lys35,Leu36,Lys37]-GLP-1-(7-37)-peptidyl-Glu-Phe-Leu amide;
[Aib8,Glu23,Val25,Glu34,Lys35,Aib36,Leu37]-GLP-1-(7-37)-peptidyl-Ser-Phe-Leu amide;
[Aib8,Glu23,Val25,Leu33,Glu34,Lys35,Aib36,Lys37]-GLP-1-(7-37)-peptidyl-Glu-Trp-Leu amide;
[Aib8,Lys23,Val25,Glu34,Lys35,Aib36,Lys37]-GLP-1-(7-37)-peptidyl-Glu-Phe-Leu amide;
[Aib8,Glu23,Val25,Arg26,Glu34,Arg35,Val36,Arg37]-GLP-1-(7-37)-peptidyl-Glu-Phe-Leu-Lys amide;
[Gly8,Glu23,Val25,Glu34,Lys35,Val36,Lys37]-GLP-1-(7-37)-peptidyl-Glu-Phe-Leu-Gly;
[Aib8,Glu23,Val25,Glu34,Lys35,Phe36,Lys37]-GLP-1-(7-37)-peptidyl-Glu-Phe-Phe amide;
[Aib8,Glu23,Val25,Gln26,Glu34,Ala35,Val36,Ala37]-GLP-1-(7-37)-peptidyl-Glu-Phe-Leu amide;
[Aib8,Glu23,Val25,Gln26,Glu34,Gln35,Aib36,Gln37]-GLP-1-(7-37)-peptidyl-Glu-Phe-Leu amide;
[Aib8,Glu23,Lys24,Val25,Glu30,His31,Glu34,Lys35,Phe36,Lys37]-GLP-1-(7-37)-peptidyl-Glu-Phe-Phe amide;
[Aib8,Glu23,Val25,Glu34,Lys35,Aib36,Lys37]-GLP-1-(7-37)-peptidyl-Glu-Phe-Leu-Glu-Lys-Val-Lys-Glu-Phe;
[Aib8,Glu23,Val25,Glu34,Lys35,Aib36,Lys37]-GLP-1-(7-37)-peptidyl-Glu-Phe-Leu-Glu-Lys-Val;
[Aib8,Glu23,Val25,Glu34,Lys35,Aib36,Lys37]-GLP-1-(7-37)-peptidyl-Glu-Phe-Leu-Glu-Lys;
[Aib8,Glu23,Val25,Glu34,Lys35,Aib36,Lys37]-GLP-1-(7-37)-peptidyl-Glu-Phe-Leu-Glu;
[Aib8,Pro22,Glu23,Val25,Glu34,Lys35,Aib36,Lys37]-GLP-1-(7-37)-peptidyl-Glu-Phe-Leu amide;
[Aib8,Val25,Gln27,Gln34,Lys35,Aib36,Lys37]-GLP-1-(7-37)-peptidyl-Gln-Phe-Leu amide;
[Lys24,Glu34,Val36,Lys37]-GLP-1-(7-37)-peptidyl-Glu-Phe-Leu amide;
[Glu34,Lys35,Val36,Lys37]-GLP-1-(7-37)-peptidyl-Glu-Phe-Leu amide;
[Glu34,Lys35,Val36,Lys37]-GLP-1-(7-37)-peptidyl-Glu-Phe-His amide;
[Glu34,Lys35,Val36,Lys37]-GLP-1-(7-37)-peptidyl-Glu-Phe-Leu-Glu amide;
[Glu34,Lys35,Val36,Lys37]-GLP-1-(7-37)-peptidyl-Glu-His-Leu amide;
[Glu34,Lys35,Val36]-GLP-1-(7-37)-peptidyl-Glu-Phe-Leu amide;
[Aib8,Glu22,Glu23,Val25,Glu34,Lys35,Val36,Lys37]-GLP-1-(7-37)-peptidyl-Glu-Leu-Leu amide;
[Aib8,Glu23,Val25,Glu34,Lys35,Val36,Lys37]-GLP-1-(7-37)-peptidyl-Glu-Lys amide;
[Gly8,Glu22,Glu23,Val25,Glu34,Lys35,Val36,Lys37]-GLP-1-(7-37)-peptidyl-Glu-Lys-Leu amide;
[Aib8,Glu23,Val25,Glu30,Glu34,Lys35,Val36,Lys37]-GLP-1-(7-37)-peptidyl-Glu-Lys-Leu amide;
[Aib8,Glu23,Val25,Glu34,Lys35,Gly36,Lys37]-GLP-1-(7-37)-peptidyl-Glu-Phe-Leu amide;
[Aib8,Glu23,Val25,Glu34,Lys35,Val36,Lys37]-GLP-1-(7-37)-peptidyl-Asp-Phe-Leu amide;
[Aib8,Glu23,Val25,Glu34,Lys35,Val36,Lys37]-GLP-1-(7-37)-peptidyl-Gly-Phe-Leu amide;
[Aib8,Glu23,Val25,Glu34,Lys35,Val36,Lys37]-GLP-1-(7-37)-peptidyl-Glu-Glu-Leu amide;
[Aib8,Glu23,Val25,Glu34,Lys35,Val36,Lys37]-GLP-1-(7-37)-peptidyl-Glu-Gly-Leu amide;
[Aib8,Glu23,Val25,Glu34,Lys35,Val36,Lys37]-GLP-1-(7-37)-peptidyl-Glu-Lys-Leu amide;
[Aib8,Glu23,Val25,Glu34,Lys35,Val36,Lys37]-GLP-1-(7-37)-peptidyl-Glu-Leu-Leu amide;
[Aib8,Glu23,Val25,Glu34,Lys35,Val36,Lys37]-GLP-1-(7-37)-peptidyl-Glu-Val-Leu amide;
[Aib8,Glu23,Val25,Glu34,Lys35,Val36,Lys37]-GLP-1-(7-37)-peptidyl-Glu-Phe-Phe amide;
[Glu34,Lys35,Val36,Lys37]-GLP-1-(7-37)-peptidyl-Glu-Phe amide;
[Glu23,Val25,Glu34,Lys35,Val36,Lys37]-GLP-1-(7-37)-peptidyl-Glu-Phe-Leu-Glu-Lys;
[Glu34,Lys35,Val36,Lys37]-GLP-1-(7-37)-peptidyl-Glu-Phe-Leu-Glu-Lys;
[Glu23,Val25,Glu34,Lys35,Val36,Lys37]-GLP-1-(7-37)-peptidyl-Glu-His-Leu-Glu-Lys;
[Glu34,Lys35,Val36,Lys37]-GLP-1-(7-37)-peptidyl-Glu-His-Leu-Glu-Lys;
[Glu34,Val36,Lys37]-GLP-1-(7-37)-peptidyl-Glu-Phe-Leu-Glu-Lys;
[Glu34,Lys35,Val36]-GLP-1-(7-37)-peptidyl-Glu-Phe-Leu-Glu-Lys;
[Glu34,Lys35,Val36,Lys37]-GLP-1-(7-37)-peptidyl-Glu-Phe-Leu-Asp-Lys;
[Glu34,Lys35,Val36,Lys37]-GLP-1-(7-37)-peptidyl-Glu-Phe-Leu-Glu-Arg;
[Glu34,Lys35,Val36,Lys37]-GLP-1-(7-37)-peptidyl-Glu-Phe-Tyr amide;
[Glu22,Glu23,Val25,Glu34,Lys35,Val36,Lys37]-GLP-1-(7-37)-peptidyl-Glu-Phe amide;
[Gly8,Glu22,Glu23,Val25,Glu34,Lys35,Val36,Lys37]-GLP-1-(7-37)-peptidyl-Glu-Phe amide;
[Aib8,Glu22,Glu23,Val25,Glu34,Lys35,Val36,Lys37]-GLP-1-(7-37)-peptidyl-Glu-Phe amide;
[Glu22,Glu23,Val25,Glu34,Lys35,Val36,Lys37]-GLP-1-(7-37)-peptidyl-Glu-Phe-Leu amide;
[Gly8,Glu22,Glu23,Val25,Glu34,Lys35,Val36,Lys37]-GLP-1-(7-37)-peptidyl-Glu-Phe-Leu amide;
[Aib8,Glu22,Glu23,Val25,Glu34,Lys35,Val36,Lys37]-GLP-1-(7-37)-peptidyl-Glu-Phe-Leu amide;
[Glu23,Val25,Arg26,Glu34,Arg35,Val36,Arg37]-GLP-1-(7-37)-peptidyl-Glu-Phe-Leu-Glu-Lys;
[Glu23,Val25,Arg26,His34,Arg35,Val36,Arg37]-GLP-1-(7-37)-peptidyl-Glu-Phe-Leu-Glu-Lys;
[Glu34,His35,Val36,Lys37]-GLP-1-(7-37)-peptidyl-Glu-Phe-Leu amide; and
[Glu34,Lys35,Val36,His37]-GLP-1-(7-37)-peptidyl-Glu-Phe-Leu amide.

15. A pharmaceutical composition comprising a therapeutically effective amount of the GLP-1 receptor agonist peptide according to claim 1 in combination with one or more pharmaceutically acceptable carriers or diluents.

16-18. (canceled)

19. A method for treating a patient having diseases or states associated with dyslipidemia, inflammation and vascular disorder, such as cardiovascular disease, endothelial dysfunction, hyperlipidemia, hypertriglyceridemia, hypercholesterolemia, hyperlipoproteinemia, HDL deficiency, apoA-I deficiency, coronary artery disease, atherosclerosis, hypertension, stroke, ischemia, infarction, myocardial infarction, hemorrhage, periheralperiferal vascular disease, restenosis, acute coronary syndrome, or reperfusion myocardial injury, macrovascular disorder and microvascular disorder; or treating, in a diabetes patient, a disease or state selected from cardiovascular disease, endothelial dysfunction, a macrovascular disorder, microvascular disorder, atherosclerosis and hypertension, said method comprising administering a pharmaceutically active amount of the GLP-1 receptor agonist peptide according to claim 1.
